(19) United States
(12) Patent Application Publication
(10) Pub. No.: US 2025/0042957 A1
(43) Pub. Date: Feb. 6, 2025

(54) DESIGNED REPEAT DOMAINS WITH DUAL BINDING SPECIFICITY AND THEIR USE

(71) Applicant: Molecular Partners AG, Zürich-Schlieren (CH)

(72) Inventors: Simon Fontaine, Zürich-Schlieren (CH); Andreas Bosshart, Zürich-Schlieren (CH); Bernd Schlereth, Zürich-Schlieren (CH); Daniel Steiner, Zürich-Schlieren (CH); Marcel Walser, Zürich-Schlieren (CH)

(73) Assignee: Molecular Partners AG, Zürich-Schlieren (CH)

(21) Appl. No.: 18/719,008

(22) PCT Filed: Dec. 14, 2022
(86) PCT No.: PCT/EP2022/085794

§ 371 Date: Jun. 12, 2024

(30) Foreign Application Priority Data
Dec. 14, 2021 (EP) 21214519.7

(51) Int. Cl. C07K 14/47; A61K 38/00; A61K 45/06

(52) U.S. Cl. CPC C07K 14/47; A61K 45/06; A61K 38/00; C07K 2318/20

(57) ABSTRACT

The present invention relates to a method of producing a designed repeat domain which has binding specificity for two targets, wherein binding of such repeat domain to its two targets is mutually exclusive. Said designed repeat domain is preferably a designed ankyrin repeat domain. The invention also provides such designed repeat domains and recombinant proteins comprising such repeat domains, as well as the use of such repeat domains as molecular switches, such as, e.g., switches to control activation or deactivation of a therapeutic agent. The invention further relates to nucleic acids encoding such repeat domains or recombinant proteins, pharmaceutical compositions comprising such recombinant proteins, repeat domains or nucleic acids, recombinant expression vectors and host cells, and the use of such proteins, nucleic acids or pharmaceutical compositions in methods for treating diseases, such as cancer.

### CROSS-REFERENCE TO RELATED APPLICATIONS

The present application claims the benefit of priority to EP21214519, filed on Dec. 14, 2021. The disclosures of this patent application are incorporated herein for all purposes by reference in their entirety.

### FIELD OF THE DISCLOSURE

The present invention relates to a method of producing a designed repeat domain which has binding specificity for two targets, wherein binding of such repeat domain to its two targets is mutually exclusive. Said designed repeat domain is preferably a designed ankyrin repeat domain. The invention also provides such designed repeat domains and recombinant proteins comprising such repeat domains, as well as the use of such repeat domains as molecular switches, such as, e.g., switches to control activation or deactivation of a therapeutic agent. The invention further relates to nucleic acids encoding such repeat domains or recombinant proteins, pharmaceutical compositions comprising such recombinant proteins, repeat domains or nucleic acids, recombinant expression vectors and host cells, and the use of such proteins, nucleic acids or pharmaceutical compositions in methods for treating diseases, such as cancer.

### BACKGROUND

Finding the optimal balance between drug efficacy and safety is an ongoing challenge in the development of targeted drugs. Numerous strategies have been explored to improve precision and specificity of drugs and herewith limit their adverse side effects. Although some strategies may have reached pre-clinical proof-of-concept, many of them face barriers when transitioning to clinical application. Thus, finding mechanisms to reliably control the rate, time and/or place of therapeutic activity of drug molecules in the body remains very desirable.

Antibodies and their intrinsic affinity and selectivity properties are extensively used in therapy. Enabling antibodies to be activatable under certain conditions can offer a layer of control to focus their therapeutic effect at a desired site of action. To do so, antibodies can be engineered to become responsive to endogenous or exogenous stimuli such as light, temperature, pH, enzymatic activity, ions, effector molecules and combination of antigens (Lucchi, R. et al., 2021, ACS Central Science, 7 (5), 724-738).

The tumor microenvironment (TME) offers a range of biochemical cues that can potentially be exploited as triggers for drug activation. An antibody on/off switch mechanism sensing the level of extracellular adenosine triphosphate (ATP), which is distinctively elevated in the TME compared to healthy tissue, has for instance been developed. Such Switch Antibody™ (or Switch-Ig®) molecules have been reported to start binding their target in the presence of 10 mM ATP and consistently showed comparable activity to non-switch control antibodies at 100 mM ATP and negligible activity at 1 mM ATP. Their production nevertheless requires a time- and labor-consuming procedure, which includes identifying an anti-ATP antibody to be used as a library template, designing and constructing a synthetic library of antibodies with an ATP-binding motif in their complementarity-determining regions (CDRs), and identifying an ATP-dependent antigen-binding antibody by phage panning (Mimoto, F. et al., 2020, Cell Reports, 33 (12), 108542).

Further approaches to drug activation switches have been developed in the context of chimeric antigen receptor (CAR) T-Cell therapy (Yu, S. et al., 2019, Molecular Cancer, 18 (1), 1-13). On-switches for CAR have been designed in which the activation of the engineered T-cell depends on the presence of a target antigen and a small molecule triggering a heterodimerization of the CAR modules. Neither the small molecule nor the antigen alone activates the CAR T-cell. These small molecule-dependent on-switch approaches can alleviate toxicity by controlling the timing, location, and dosage of T cell activity (Wu, C. Y. et al., 2015, Science, 350 (6258); Zajc, C. U. et al., 2020, PNAS, 117 (26), 14926-14935). Off-switch mechanisms can also be implemented, by relying on protein degradation regulation. An example of such off-switch depends on lenalidomide (Revlimid®) which can induce CAR degradation. To turn off CAR signaling, lenalidomide induces ubiquitination and subsequent proteasomal degradation of the degron-tagged CAR. As levels of the small molecule fall, CAR signaling can be restored by translation of the CAR protein (Jan, M. et al., 2021, Science Translational Medicine, 13 (575); WO 2019/089592 A1). However, such tailored mechanisms to mitigate adverse effects in adoptive cell therapy cannot always be applied in non-cellular treatment modalities.

Another control mechanism is provided by reversible on/off molecular switches based on allosteric regulation. Allosteric proteins can act as molecular switches in which binding of a molecule to a site different from the protein's active site changes the conformation of said protein. Although more prevalent as biosensors, modular allosteric switches have been designed to control the activity of proteins through effector molecules. In one such design, a synthetic dual ligand molecule having mutually exclusive binding sites is fused through a linker to the protein of interest. In absence of the effector molecule, the dual ligand binds the active site of the protein, thereby inhibiting the protein's activity. In presence of the effector, binding of the effector to the ligand molecule releases the inhibitor of the protein, thereby activating the protein (Schena, A. et al., 2015, Nature Communications, 6 (1), 1-10; WO 2015/067302 A1). Applying this approach to a therapeutically relevant target is however limited by the requirement of selecting or identifying specific allosteric inhibitor molecules for the target which is to be switched on/off. Further limitations may come from design requirements of the fused dual ligand, which in this particular case includes a fluorescent dye.

A modular protein switch comprising alternative binding scaffolds (monobody and designed ankyrin repeat protein (or DARPin)) has also been proposed (Nicholes, N. et al., 2016, Protein Engineering, Design and Selection, 29 (2), 77-85; WO 2003/078575 A2). In this approach, a binding scaffold comprises an input domain, such as a DARPin having binding affinity for Maltose Binding Protein (MBP), and further comprises an output domain, such as a β-lactamase enzyme which is fused into a selected site of the input domain, such as between the first and second internal repeats of the DARPin. Although some expected switch activity (i.e. a scaffold binding ligand-dependant enzymatic activity of β-lactamase) was observed, the fused DARPins, which exhibited switch properties, suffered significant loss of affinity for their ligand, as compared to the corresponding non-fused DARPins. Also, when attempting to generalize the applicability of these switch constructs to further ligands by mutagenesis, only limited success was observed in redirecting the specificity of the binding scaffolds.

Taken together, currently available molecular drug switch approaches are time-, cost- and/or labor-consuming, and limited in their capabilities and applications. Thus, there remains a need for new molecular switch designs. Such switch designs may be useful in the control of protein activity, e.g. in protein-based therapeutics.

### SUMMARY

The present invention provides a method of producing a designed repeat domain which has binding specificity for two targets, and wherein the binding of said domain to its both targets is mutually exclusive. Said designed repeat domain is preferably a designed ankyrin repeat domain. The invention also provides such designed ankyrin repeat domains and recombinant proteins comprising such repeat domains, as well as the use of such repeat domains as molecular switches, such as, e.g., switches to control activation or deactivation of a therapeutic agent. In addition, the invention provides nucleic acids encoding such repeat domains or recombinant proteins, pharmaceutical compositions comprising such recombinant proteins, repeat domains or nucleic acids, recombinant expression vectors and host cells, and the use of such proteins, nucleic acids or pharmaceutical compositions in methods for treating diseases, such as cancer in a mammal, including a human.

In one aspect, the invention provides a method of producing a designed repeat domain, the method comprising the steps of:
(i) providing a first repeat domain and a second repeat domain, wherein said first repeat domain has binding specificity for a first target and said second repeat domain has binding specificity for a second target; and
(ii) joining part of said first repeat domain and part of said second repeat domain to form a designed repeat domain with dual binding specificity, by covalently combining at least one repeat module of said first repeat domain and at least one repeat module of said second repeat domain;
   wherein said designed repeat domain has binding specificity for said first target and binding specificity for said second target, and wherein binding of said designed repeat domain to said first and second targets is mutually exclusive.

In another aspect, the invention provides a designed repeat domain obtainable by the above method.

In another aspect, the invention provides a designed repeat domain having a first binding specificity for a first target and a second binding specificity for a second target, and wherein the binding of said repeat domain to the first and second targets is mutually exclusive.

Designed repeat domains of the invention bind their two targets only in a mutually exclusive manner, i.e. upon binding of the repeat domain to one target molecule, the binding of said domain to the other target molecule is prevented, mainly due to steric hindrance effects.

Repeat domains of the present invention can be advantageously used as molecular on/off switch mechanism enabling a conditional activation or deactivation of a therapeutic agent's biological activity. Such biological activity of the therapeutic agent may be, for example, the binding of the therapeutic agent to a biological target. In such a scenario, the repeat domain of the invention is chemically linked to the therapeutic agent, and the first target molecule of said repeat domain is the therapeutic agent, while the second target molecule is a sensor target molecule. Preferably such sensor target molecule is related to a pathological condition, e.g. a molecule that is selectively expressed or overexpressed in a pathological condition. One such pathological condition is cancer, and said sensor target molecule is selectively expressed or overexpressed in tumor tissue.

Accordingly, in another aspect, the invention provides recombinant proteins comprising such designed repeat domain of the invention. In one particular aspect, such recombinant proteins comprising such designed repeat domain of the invention further comprise a therapeutic agent. In one aspect, the therapeutic agent is a binding molecule. In a further aspect, said binding molecule comprises an ankyrin binding domain.

The state (active/on or inactive/off) of a therapeutic agent in a recombinant protein of the invention depends on the binding of the designed repeat domain of the invention to its first and second targets. In one aspect, in the on-state, the designed repeat domain of the invention cannot bind its first target (e.g., the therapeutic agent) due to the second target (e.g., a sensor target) which is present and substantially bound to the designed repeat domain (see, e.g. on-switch in FIG. 3A). The first target (e.g., therapeutic agent) is therefore unbound and active. In the off-state, practically no second target is present and consequently the designed repeat domain binds the first target (e.g., the therapeutic agent) herewith preventing its normal activity.

In another aspect, the invention provides recombinant proteins comprising such designed repeat domain of the invention which do not further comprise a therapeutic agent. The state (active/on or inactive/off) of such a recombinant protein of the invention may also depend on the binding of the designed repeat domain of the invention to its first and second targets. In the on-state, a designed repeat domain of the invention can bind its first target (e.g., a cell surface receptor) due to the second target (e.g., a sensor target) not being present in substantial amounts and hence not bound to the designed repeat domain (see, e.g. off-switch in FIG. 3B). The first target (e.g., a cell surface receptor) is therefore bound by the designed repeat domain of the invention and thereby activated or inhibited in its biological activity. In the off-state, the second target (e.g. a sensor target) is present in substantial amounts and hence bound to the designed repeat domain, thereby preventing binding of the designed repeat domain to the first target.

In another aspect, the invention provides isolated nucleic acids encoding a designed repeat domain of the invention or encoding a recombinant protein of the invention, a recombinant expression vector comprising such nucleic acids, host cells comprising such expression vectors and pharmaceutical compositions comprising the designed repeat protein, recombinant protein, nucleic acid, recombinant expression vector and/or host cell of the invention as well as at least one pharmaceutically acceptable carrier or diluent.

In another aspect, the invention provides a method of treating a medical condition, the method comprising the step of administering to a patient in need thereof a therapeutically effective amount of the designed repeat domain, recombinant protein, nucleic acid, or pharmaceutical composition of the invention. In one particular aspect, said medical condition is a cancer.

In another aspect, the invention provides the designed repeat domain, recombinant protein, nucleic acid, or pharmaceutical composition of the invention for use in a method of treating a medical condition. In one particular aspect, said medical condition is a cancer.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A method of producing a designed repeat domain, the method comprising the steps of:
(i) providing a first repeat domain and a second repeat domain, wherein said first repeat domain has binding specificity for a first target and said second repeat domain has binding specificity for a second target; and
(ii) joining part of said first repeat domain and part of said second repeat domain to form a designed repeat domain with dual binding specificity, by covalently combining at least one repeat module of said first repeat domain and at least one repeat module of said second repeat domain;
wherein said designed repeat domain has binding specificity for said first target and binding specificity for said second target, and wherein binding of said designed repeat domain to said first and second targets is mutually exclusive.

E2. The method of E1, wherein said covalently combining at least one repeat module of said first repeat domain and at least one repeat module of said second repeat domain comprises covalently connecting said at least one repeat module of said first repeat domain and said at least one repeat module of said second repeat domain.

E2.1. The method E2, wherein said at least one repeat module of said first repeat domain and said at least one repeat module of said second repeat domain are covalently connected by a linker.

E2.2. The method of E2.1, wherein said linker is a peptide linker or a non-binding repeat module.

E3. The method of E1, wherein said covalently combining at least one repeat module of said first repeat domain and at least one repeat module of said second repeat domain comprises covalently merging said at least one repeat module of said first repeat domain and said at least one repeat module of said second repeat domain.

E4. The method of any one of E1 to E3, wherein said designed repeat domain comprises an N-terminal capping module and/or a C-terminal capping module, and at least one internal repeat module,

E5. The method of E4, wherein said N-terminal capping module is at least 90% identical in amino acid sequence to an N-terminal capping module comprised in said first repeat domain and said C-terminal capping module is at least 90% identical in amino acid sequence to a C-terminal capping module comprised in said second repeat domain.

E6. The method of any one of E1 to E5, wherein said part of said first repeat domain comprises an N-terminal capping module and at least one repeat module, and wherein said part of said second repeat domain comprises a C-terminal capping module and at least one repeat module.

E7. The method of any one of E1 to E6, wherein an N-terminal fragment of said first repeat domain is covalently combined with a C-terminal fragment of said second repeat domain.

E8. The method of any one of E1 to E7, wherein step (ii) comprises removing a C-terminal capping module of said first repeat domain and removing an N-terminal capping module of said second repeat domain, such that said C-terminal capping module of said first repeat domain and said N-terminal capping module of said second repeat domain are not comprised in said designed repeat domain.

E9. The method of any one of E1 to E8, wherein said designed repeat domain, when bound to said first target or said second target, inhibits a biological activity of the bound target.

E10. The method of any one of E1 to E9, wherein binding of said designed repeat domain to one of said first and second targets sterically hinders binding of said designed repeat domain to the other of said first and second targets.

E11. The method of any one of E1 to E10, wherein said designed repeat domain binds to said first target with a dissociation constant (KD) below 10⁻⁵M.

E12. The method of any one of E1 to E11, wherein said designed repeat domain binds to said second target with a dissociation constant (KD) below 10⁻⁵M.

E13. The method of any one of E1 to E12, wherein said designed repeat domain binds to one of said first and second targets with a first binding affinity and to the other of said first and second targets with a second binding affinity, wherein the ratio of said first binding affinity and said second binding affinity is between 1:1 and 1:105.

E14. The method of any one of E1 to E13, wherein said first and second targets are located on different molecules.

E15. The method of any one of E1 to E14, wherein said first repeat domain is an ankyrin repeat domain and said second repeat domain is an ankyrin repeat domain.

E16. The method of any one of E1 to E15, wherein said designed repeat domain is an ankyrin repeat domain.

E17. A designed repeat domain obtainable by the method of any one of the preceding embodiments.

E18. A designed repeat domain having a first binding specificity for a first target and a second binding specificity for a second target, wherein binding of said designed repeat domain to said first and second targets is mutually exclusive.

E19. The designed repeat domain of E18, wherein the designed repeat domain, when bound to said first target or said second target, inhibits a biological activity of the bound target.

E20. The designed repeat domain of any one of E18 to E19, wherein binding of said designed repeat domain to one of said first and second targets sterically hinders binding of said designed repeat domain to the other of said first and second targets.

E21. The designed repeat domain of any one of E18 to E20, wherein said designed repeat domain binds to said first target with a dissociation constant (KD) below 10⁻⁵M.

E22. The designed repeat domain of any one of E18 to E21, wherein said designed repeat domain binds to said second target with a dissociation constant (KD) below 10⁻⁵M.

E23. The designed repeat domain of any one of E18 to E22, wherein said designed repeat domain binds to one of said first and second targets with a first binding affinity and to the other of said first and second targets with a second binding affinity, wherein the ratio of said first binding affinity and said second binding affinity is between 1:1 and 1:105.

E24. The designed repeat domain of any one of E18 to E23, wherein said first and second targets are located on different molecules.

E25. The designed repeat domain of any one of E18 to E24, wherein said designed repeat domain comprises at least three repeat modules.

E26. The designed repeat domain of any one of E18 to E25, wherein said designed repeat domain comprises an N-terminal capping module and/or a C-terminal capping module, and at least one internal repeat module, at least two internal repeat modules, at least three internal repeat modules or at least four internal repeat modules.

E27. The designed repeat domain of any one of E18 to E26, wherein all internal repeat modules comprised in said designed repeat domain contribute to the first and/or second binding specificity.

E28. The designed repeat domain of any one of E18 to E27, wherein said designed repeat domain comprises at least one internal repeat module which contributes to binding to said first target, and wherein said designed repeat domain comprises at least one internal repeat module which contributes to binding to said second target.

E29. The designed repeat domain of any one of E18 to E28, wherein the designed repeat domain is an ankyrin repeat domain.

E30. The designed repeat domain of any one of E18 to E29, wherein said designed repeat domain comprises at least one, at least two, at least three, at least four, or at least five target interaction residues which bind to said first target, and wherein said designed repeat domain comprises at least one, at least two, at least three, at least four, or at least five target interaction residues which bind to said second target.

E31. The designed repeat domain of any one of E18 to E30, wherein the designed repeat domain comprises at least one internal repeat module comprising a sequence selected from SEQ ID NOs: 75 to 81.

E32. The designed repeat domain of any one of E18 to E31, wherein said designed repeat domain comprises (i) an N-terminal capping module comprising a sequence selected from the group consisting of (1) SEQ ID NOS: 35, 36, 47 to 63 and 83 to 85 and (2) sequences in which up to 9 amino acids in any of SEQ ID NOs: 35, 36, 47, 53, 59, 60, 83 and 84 are substituted by another amino acid; and/or (ii) a C-terminal capping module comprising a sequence selected from the group consisting of (1) SEQ ID NOS: 37, 38 and 64 to 74 and (2) sequences in which up to 9 amino acids in any of SEQ ID NOs: 37, 38 and 69 are substituted by another amino acid.

E32.1. The designed repeat domain of any one of E18 to E32, wherein said designed repeat domain comprises (i) an N-terminal capping module comprising a sequence selected from the group consisting of (1) SEQ ID NOs: 35, 36, 47 to 63, 83 to 85 and 97 to 100 and (2) sequences in which up to 9 amino acids in any of SEQ ID NOs: 35, 36, 47, 53, 59, 60, 83 and 84 are substituted by another amino acid; and/or (ii) a C-terminal capping module comprising a sequence selected from the group consisting of (1) SEQ ID NOs: 37, 38 and 64 to 74 and (2) sequences in which up to 9 amino acids in any of SEQ ID NOs: 37, 38 and 69 are substituted by another amino acid, and/or (iii) at least one internal repeat module comprising a sequence selected from the group consisting of (1) SEQ ID NOs: 40 to 46 and (2) sequences in which up to 9 amino acids in any of SEQ ID NOs: 40 to 46 are substituted by another amino acid.

E33. The designed repeat domain of any one of E18 to E32.1, wherein said designed repeat domain comprises at least one internal repeat module comprising a sequence selected from the group consisting of (1) SEQ ID NOs: 40 to 46 and (2) sequences in which up to 9 amino acids in any of SEQ ID NOs: 40 to 46 are substituted by another amino acid.

E34. The designed repeat domain of any one of E18 to E33, wherein said designed repeat domain comprises (i) an N-terminal capping module comprising a sequence selected from the group consisting of (1) SEQ ID NOS: 35 to 36 and (2) sequences in which up to 9 amino acids in any of SEQ ID NOs: 35 to 36 are substituted by another amino acid; and/or (ii) a C-terminal capping module comprising a sequence selected from the group consisting of (1) SEQ ID NO: 38 and (2) sequences in which up to 9 amino acids in SEQ ID NO: 38 are substituted by another amino acid.

E35. The designed repeat domain of any one of E18 to E34, wherein said designed repeat domain comprises at least one internal repeat module, wherein residues at positions corresponding to positions 3, 4, 6, 11, 14 and/or 15 of SEQ ID NO: 39 are target interaction residues.

E36. The designed repeat domain of any one of E18 to E35, wherein said designed repeat domain comprises at least one N-terminal capping module, wherein residues at positions corresponding to positions 4, 5, 8, 11 and/or 12 of SEQ ID NO: 36 are target interaction residues.

E37. The designed repeat domain of any one of E18 to E36, wherein said designed repeat domain comprises at least one C-terminal capping module, wherein residues at positions corresponding to positions 3, 4, 6, 14 and/or 15 of SEQ ID NO: 38 are target interaction residues.

E38. The designed repeat domain of any one of E18 to E37, wherein said designed repeat domain comprises two internal repeat modules, wherein the N-terminal one of said two internal repeat modules comprises target interaction residues which bind to said first target, and wherein the C-terminal one of said two internal repeat modules comprises target interaction residues which bind to said second target.

E39. The designed repeat domain of any one of E18 to E37, wherein said designed repeat domain comprises three internal repeat modules, wherein the most N-terminal one of said three internal repeat modules comprises target interaction residues which bind to said first target, and wherein the most C-terminal one of said three internal repeat modules comprises target interaction residues which bind to said second target, and wherein the middle one of said three internal repeat modules comprises target interaction residues which bind to said first and/or second target(s).

E40. The designed repeat domain of E39, wherein the designed repeat domain comprises an amino acid sequence selected from the group consisting of (1) SEQ ID NOs: 6 to 10 and (2) sequences with at least 80% amino acid sequence identity with any one of SEQ ID NOs: 6 to 10.

E41. The designed repeat domain of any one of E18 to E37, wherein said designed repeat domain comprises four internal repeat modules, wherein the most N-terminal one of said four internal repeat modules comprises target interaction residues which bind to said first target, wherein the most C-terminal one of said four internal repeat modules comprises target interaction residues which bind to said second target, and wherein each of the second-most N-terminal one and the second-most C-terminal one of said four internal repeat modules comprises target interaction residues which bind to said first target and/or said second target.

E42. The designed repeat domain of E41, wherein the designed repeat domain comprises an amino acid sequence selected from the group consisting of (1) SEQ ID NOS: 11 to 13 and (2) sequences with at least 80% amino acid sequence identity with any one of SEQ ID NOs: 11 to 13.

E43. A recombinant protein comprising the designed repeat domain according to any one of claims 17 to 42.

E44. The protein according to E43, wherein the protein further comprises a therapeutic agent.

E45. The protein according to E44, wherein the therapeutic agent is a binding molecule.

E46. The protein according to E45, wherein the binding molecule comprises a designed ankyrin repeat domain.

E47. The protein according to E45 or E46, wherein the binding molecule is a T cell engager.

E48. An isolated nucleic acid encoding the designed repeat domain according to any one of E17 to E42 or the protein according to any one of E43 to E47.

E49. A recombinant expression vector comprising the nucleic acid according to E48.

E50. A host cell comprising the recombinant expression vector according to E49.

E51. A pharmaceutical composition comprising one or more of: (i) the designed repeat domain according to any one of E17 to E42, (ii) the recombinant protein according to any one of E43 to E47, (iii) the nucleic acid according to E48, and/or (iv) the recombinant expression vector according to E49, and optionally a pharmaceutically acceptable carrier or diluent.

E52. A method of treating a medical condition, the method comprising the step of administering to a patient in need thereof a therapeutically effective amount of the designed repeat domain according to any one of E17 to E42, the recombinant protein according to any one of E43 to E47, the nucleic acid of E48 or the pharmaceutical composition of E51.

E53. The method of E52, wherein said medical condition is a cancer.

E54. The designed repeat domain according to any one of E17 to E42, the recombinant protein according to any one of E43 to E47, the nucleic acid of E48 or the pharmaceutical composition according to E51, for use in a method of treating a medical condition.

E55. The designed repeat domain, the recombinant protein, the nucleic acid or the pharmaceutical composition for use according to E54, wherein said medical condition is a cancer.

### BRIEF DESCRIPTION OF THE FIGURES

FIG. 1: Illustration of designed repeat domains of the invention, as exemplified by designed ankyrin repeat domains of the invention, also termed “2-in-1 DARPins” or “SwitchDARPins” (6). DARPins typically comprise an N-terminal cap (N-cap), one or more internal repeat modules (most commonly one, two or three), and a C-terminal cap (C-cap). The target-(or antigen-) binding paratope (or binding surface) (4, 5) is a continuous surface formed by one or several of these repeat modules. A 2-in-1 DARPin can be formed by genetically combining the N-cap plus one or several internal repeat modules of a first DARPin binding a target A with the C-cap plus one or several internal repeat modules from a second DARPin binding a target B (the two parent DARPins are shown as (1) and (2)). The resulting 2-in-1 DARPin (6) then typically contains a N-cap, a C-cap, and at least one internal repeat module, most commonly two internal repeat modules (termed N2C), three internal repeat modules (N3C), or four internal repeat modules (N4C). It may contain even more internal repeat modules (N×C). The 2-in-1 DARPin is able to bind target A and target B, but not simultaneously (see FIG. 2). Additionally, one or several internal repeat modules in the 2-in-1 DARPin can be mixed (or merged) repeat modules (7). A repeat module is called a mixed repeat module if its binding surface comprises at least one target interaction residue from the corresponding module of the first parent repeat protein and at least one target interaction residue from the corresponding module of the second parent repeat protein.

FIG. 2: The concept of mutually exclusive binding is illustrated. Due to the close proximity or overlap (overlap is represented as diagonally striped) of the two paratopes of the 2-in-1 DARPin, the molecule is able to bind target A (FIG. 2A) or target B (FIG. 2B) individually, but not both targets at the same time (FIG. 2C), as the simultaneous binding of both targets A and B is sterically hindered. The steric clash is represented by the star symbol between target A and target B.

FIG. 3: Generic examples of constructs comprising a 2-in-1 DARPin of the invention. These exemplary constructs are also referred to as “Switchdrug DARPins”. In FIG. 3A, an exemplary on-switch mechanism is shown, where a 2-in-1 DARPin binds target A (e.g. a sensor target) or target B (e.g. a drug molecule) in a mutually exclusive manner and is chemically linked to said target B (e.g. a drug molecule). The state of the target B (e.g. active or inactive drug molecule) as part of the Switchdrug DARPin is conditionally regulated by the interaction of the 2-in-1 DARPin with target A, wherein binding of target A by the 2-in-1 DARPin results in release of bound target B (hence, active drug molecule). In an exemplary off-switch setup shown in FIG. 3B, a 2-in-1 DARPin is designed to bind target A (e.g. a sensor target) or target B (e.g. a cell receptor). A downstream activity resulting from the interaction of said 2-in-1 DARPin with target B can be conditionally switched-off upon binding of target A to the 2-in-1 DARPin. In such Switchdrug DARPins the 2-in-1 DARPin can be further linked to other binding domains or molecules.

FIG. 4: Exemplary designs of Switchdrug DARPins according to the invention as on- and off-switches to conditionally regulate a T-cell engager (TCE). In the on-switch concept (FIG. 4A), the T-cell engager comprises a CD3-binding DARPin (target B) and a tumor-associated antigen (TAA)-binding DARPin. A 2-in-1 DARPin is used which can bind to and block the paratope of the CD3-binding DARPin, and which can alternatively bind to a molecule that is predominantly present in the tumor-microenvironment, represented as target A. This 2-in-1 DARPin is chemically linked to the TCE to form a Switchdrug DARPin. High concentrations of target A induce the release of the CD3-binding DARPin (target B), enabling the recruitment of cytotoxic T-cells and the formation of an immunological synapse between a TAA-expressing tumor cell and a T-cell. In the exemplary off-switch concept (FIG. 4B), a 2-in-1 DARPin is used which binds target A (a sensor target) and alternatively binds target B (CD3). The TCE in this case comprises a tumor-associated antigen (TAA)-binding DARPin chemically linked to said 2-in-1 DARPin. Upon high concentrations of target A, the TCE can be silenced by binding to target A, thereby preventing binding of the TCE to CD3 on T cells.

FIG. 5: Detailed representation of 2-in-1 DARPins which can be used as VEGF-triggered T-cell engager Switchdrug DARPin. 2-in-1 DARPins as represented in FIG. 1 were created by combining a first VEGF-binding DARPin, also referred to as “VEGF binder” (SEQ ID NO: 1) having high affinity towards VEGF, and a second DARPin targeting the paratope of a CD3-binding DARPin, also referred to as “α-CD3 DARPin-blocker” (SEQ ID NO: 2). Thus, by combining N-cap plus one or two internal repeat modules from the VEGF binder, and one or two internal repeat modules plus the C-cap of the α-CD3 DARPin-blocker, VEGF-regulated 2-in-1 DARPins can be generated. 5A-5B: Amino acid sequence alignments of the VEGF-binder, the α-CD3 DARPin-blocker and eight 2-in-1 DARPin designs according to the invention. FIG. 5A shows the N3C 2-in-1 DARPins (i.e. 2-in-1 DARPins 11, 12, 13, 15 and 17 corresponding to SEQ ID NOs: 6, 7, 8, 9 and 10, respectfully) and FIG. 5B shows the N4C 2-in-1 DARPins (i.e. 2-in-1 DARPins 18, 19 and 20, corresponding to SEQ ID NOs: 11, 12 and 13, respectfully). Numbers boxed with black borders in the repeat numbering row indicate paratope positions. 5C-5D: A visualization method of the paratope of DARPins is depicted, representing the surface of the DARPin paratope in 3D (FIG. 5C) and 2D (FIG. 5D) formats. FIG. 5C shows a cartoon representation of a N3C DARPin (a DARPin containing an N-cap, 3 internal repeats and a C-cap) with highlighted paratope positions in each repeat module. FIG. 5D provides a position map showing for each of the repeat modules which positions of the paratope comprise framework residues or potential target interaction residues. The dotted line in FIG. 5C or 5D illustrates the relative orientation of the same residues in the 3D and 2D representation. 5E: The paratopes of the VEGF-binder, α-CD3 DARPin-blocker and the eight 2-in-1 DARPins from FIG. 5A-5B depicted in 2D representation. The bar below the 2D representation indicates whether the repeat module was taken from the VEGF-binder, the α-CD3 DARPin-blocker, or is a mixed repeat module between α-CD3 DARPin-blocker and VEGF-binder.

FIG. 6: Surface plasmon resonance (SPR) data of the individual VEGF binder, α-CD3 DARPin-blocker and the eight 2-in-1-DARPins binding to VEGF and α-CD3 DARPin. 6A: Schematic representation of the three different types of SPR experiments of FIG. 6D. 6B: Binding of the VEGF binder to its target (VEGF165) was assessed in a single-trace SPR measurement (see U.S. Pat. No. 9,458,211B1 for detailed data). 6C: Multi-trace SPR data was recorded for the α-CD3 DARPin-blocker against the target CD3-binding DARPin, whereas no binding towards VEGF could be measured. 6D: Multi-trace SPR data of the eight 2-in1 DARPins binding immobilized VEGF or α-CD3 DARPin, as well as single-trace SPR data for binding α-CD3 DARPin in absence and presence of 130 nM soluble VEGF.

FIG. 7: Residual binding of Switchdrug DARPins to VEGF by ELISA. Data of eight His-tagged Switchdrug DARPins in two different formats binding biotinylated VEGF 165 that is bound to streptavidin-coated microtiter plates. Binding is detected via an HRP-labeled anti-His antibody. Four Switchdrug DARPins in format A ([α-CD123 binder]-[α-CD3 binder]-[2-in-1 DARPin]) and four in format B ([2-in-1 DARPin]-[α-CD123 binder]-[α-CD3 binder]) were analysed, and a VEGF-binder was used as control.

FIG. 8: Switch-property analysis. ELISA data of His-tagged Switchdrug DARPins of the invention comprising 2-in-1 DARPins 13, 15, 17 or 18 in different formats binding biotinylated scCD3εγ (bio-scCD3εγ) that is bound to streptavidin-coated microtiter plates. Binding is detected via an HRP-labeled anti-His antibody. 8A: Binding of Switchdrug DARPins 15-1, 17-1 or 18-1 (format 1) to bio-scCD3εγ in presence and absence of 100 nM VEGF. Format 1 denotes constructs formatted in the order [α-CD3 binder]-[2-in-1 DARPin]. The shift in EC50 of the Switchdrug DARPins in presence and absence of VEGF is indicated. A α-CD3 mono-DARPin in presence and absence of 100 nM VEGF served as control. 8B: Binding of Switchdrug DARPins 13-3, 15-3, 17-3 or 18-3 (format 3) to bio-scCD3εγ in presence and absence of 100 nM VEGF. Format 3 denotes constructs formatted in the order [2-in-1 DARPin]-[Ni2C]-[α-CD3 binder]. Ni2C represents a non-binding DARPin. The shift in EC50 of the Switchdrug DARPins in presence and absence of VEGF is indicated. A α-CD3 mono-DARPin in presence and absence of 100 nM VEGF served as control. 8C: Binding of Switchdrug DARPins 17-A and 18-A (format A) to bio-scCD3εγ in presence and absence of 100 nM VEGF. Format A denotes constructs formatted in the order [α-CD123 binder]-[α-CD3 binder]-[2-in-1 DARPin]. The shift in EC50 of the Switchdrug DARPins in presence and absence of VEGF is indicated. An α-CD3 mono-DARPin served as control. 8D: Binding of Switchdrug DARPins 17-B and 18-B (format B) to bio-scCD3εγ in presence and absence of 100 nM VEGF. Format B denotes constructs formatted in the order [2-in-1 DARPin]-[α-CD123 binder]-[α-CD3 binder]. The shift in EC50 of the Switchdrug DARPins in presence and absence of VEGF is indicated. An α-CD3 mono-DARPin served as control.

FIG. 9: VEGF titration analysis. ELISA data of His-tagged Switchdrug DARPins in format 1 ([α-CD3 binder]-[2-in-1 DARPin]) or 3 ([2-in-1 DARPin]-[Ni2C]-[α-CD3 binder]) binding bio-scCD3εγ that is bound to streptavidin-coated microtiter plates. 9A: Switchdrug DARPin 17-1 was titrated (left graph) or kept at constant concentration of 10 nM with soluble VEGF titrated from 100 nM to ˜0.1 nM (right graph). A VEGF-dependent binding curve of the Switchdrug DARPin 17-1 can be observed. 9B: Same as for 9A, but with Switchdrug DARPin 17-3, where Ni2C is a non-binding DARPin. The fixation of Switchdrug DARPin 17-3 and titration of VEGF resulted in a robust VEGF-dependent binding curve. 9C: Switchdrug DARPin 18-3 was titrated (left graph) or kept at constant concentration of 100 nM with soluble VEGF titrated from 500 nM to ˜0.5 nM (right graph). A robust VEGF dependent binding signal was detected.

FIG. 10: Homogeneous Time Resolved Fluorescence (HTRF) data of four Switchdrug DARPin constructs of the invention. 10A: Schematic illustration of the HTRF setup, in absence of soluble VEGF. The bio-scCD3εγ is bound by a Terbium-labeled strepatavidin molecule, whereas the Switchdrug DARPin construct is bound by a d2-labeled anti-His antibody. In absence of VEGF the Switchdrug DARPin is preferentially in its closed conformation and hence a low signal intensity is expected. 10B: Upon addition of soluble VEGF, the Switchdrug DARPin is opened and the binding signal is expected to increase. 10C: HTRF signal x-fold over PBS of Switchdrug DARPins 13-3, 15-3, 17-3 or 18-3 in absence or presence of 100 nM VEGF. A clear difference in signal intensity can be observed for all four constructs. 10D: HTRF signal (-fold over PBS) of titration of VEGF to a constant concentration of the same Switchdrug DARPins (20 nM) as in FIG. 10C.

FIG. 11: Jurkat cell binding experiment of Switchdrug DARPin 17-B in absence or presence of VEGF (100 nM or 400 nM). A control construct where the 2-in-1 DARPin is exchanged with the VEGF-binder served as control.

FIG. 12: T-cell activation assay (CD69+ cells as % of CD8+ cells) of Switchdrug DARPins 13-A, 13-B, 15-A, 15-B, 17-A, 17-B, 18-A and 18-B. As shown, format A (FIG. 12A) corresponds to: [α-CD123 binder]-[α-CD3 binder]-[2-in-1 DARPin] and format B (FIG. 12B) to: [2-in-1 DARPin]-[α-CD123 binder]-[α-CD3 binder]. Additionally, TCE control construct ([α-CD123 binder]-[α-CD3 binder]) and Switchdrug format controls ([α-CD123 binder]-[α-CD3 binder]-[α-VEGF binder] for format A and [α-VEGF binder]-[α-CD123 binder]-[α-CD3 binder] for format B) were used. T-cell activation was determined in absence and presence of 100 nM VEGF and the shift in EC50 upon addition of VEGF is indicated in each graph.

FIG. 13: Exemplary profiling method applied to assess compatibility of parent DARPins of interest with being covalently combined to form a 2-in-1 DARPin. This method is used to determine an optimal starting point for subsequent combination designs based on two parent repeat domains. The graphics show the assessment for the N-terminal side of a parent DARPin. The assessment of the C-terminal side follows the same logic and is analogous. In each of steps 1, 2 and 4, a variant of the parent DARPin comprising one or more repeat modules of a template non-binding repeat domain (such as SEQ ID NO: 5) is created and subsequently the binding of said variant to the target of the parent DARPin is tested. In step 3, a variant of the parent DARPin comprising a modified capping module and one less internal repeat modules is created and subsequently the binding of said variant to the target of the parent DARPin is tested. Further details are provided in Example 9. IR stands for internal repeat module, N stands for N-terminal capping module, and C stands for C-terminal capping module.

FIG. 14: Characterisation of 2-in-1 DARPins (i.e. 2-in-1 DARPin 21 (SEQ ID NO: 90) and 2-in-1 DARPin 22 (SEQ ID NO: 91)) having mutually exclusive binding specificities for Maltose binding protein (MBP) and Aminoglycoside phosphotransferase (APH). 14A: Size exclusion chromatography (SEC) profiles. 14B: Response curves from multi-trace SPR analysis of 2-in-1 DARPin 21, 2-in-1 DARPin 22, and parent DARPins no 1 (SEQ ID NO: 86) and 2 (SEQ ID NO:87) binding either to target APH or to target MBP as indicated. Parent DARPin no 1 binds to MBP (plot 5), and parent DARPin no 2 binds to APH (plot 6). 14C: Results of a first SPR competitive binding assay. 2-in-1 DARPin 21 and 2-in-1 DARPin 22 were pre-incubated with different concentration of soluble MBP for 40 min prior to running the samples on the APH coated SPR chip (plots 1 & 3). MBP acts as competitor for the binding of APH, as observed by the MBP-dependent response curve (plots 2 & 4). 14D: Results of a second SPR competitive binding assay. 2-in-1 DARPin 21 and 2-in-1 DARPin 22 were pre-incubated with different concentration of soluble APH for 40 min prior to running the samples on the MBP coated SPR chip (plots 1 & 3). APH acts as competitor for the binding of MBP, as observed by the APH-dependent response curve (plots 2 & 4). 14E: Results of a competitive ELISA based binding assay. 2-in-1 DARPin 21 and 2-in-1 DARPin 22 at a final concentration of 100 nM were incubated with serial dilutions of MBP for 1 hour prior to exposure to the APH target coated on an ELISA plate. MBP acts as competitor for the binding of APH, as observed by the MBP-dependent response curve. 14F: Independent binding of the 2-in-1 DARPins to target MBP (plot 1) or APH (plot 2) by ELISA. Parent DARPins no 1 and 2 were used as positive controls. 2-in-1 DARPins and parent DARPins were tested at different concentrations as indicated.

FIG. 15: Characterisation of 2-in-1 DARPins (i.e. 2-in-1 DARPin 23 (SEQ ID NO: 92), 2-in-1 DARPin 24 (SEQ ID NO: 93) and 2-in-1 DARPin 25 (SEQ ID NO: 94)) having mutually exclusive binding specificities for scCD3εγ and TargetA. 15A: Size exclusion chromatography (SEC) profiles. 15B: Response curves from multi-trace SPR analysis of 2-in-1 DARPin 23, 2-in-1 DARPin 24, 2-in-1 DARPin 25 and parent DARPins no 3 (SEQ ID NO: 3) and 4 (SEQ ID NO: 88) binding either to targets scCD3εγ or to TargetA as indicated. Parent DARPin no 4 binds to TargetA (plot 7), and parent DARPin no 3 binds to scCD3εγ (plot 8). 15C: Results of an SPR competitive binding assay. 2-in-1 DARPin 23 was pre-incubated with different concentrations of soluble TargetA for 4.5 hours prior to running the sample on the bio-scCD3εγ coated SPR chip (plot 1). TargetA acts as competitor for the binding of scCD3εγ, as observed by the TargetA-dependent response curve (plot 2). 15D: Mutual exclusivity of binding of the 2-in-1 DARPins to their two targets can alternatively be measured by a competitive binding assay based on HTRF. Binding of 2-in-1 DARPin 23, 2-in-1 DARPin 24 and 2-in-1 DARPin 25 to bio-scCD3εγ in presence of different concentrations of non-biotinylated TargetA competitor was assessed. A TargetA concentration-dependent reduction of binding to scCD3εγ is observed for all tested 2-in-1 DARPins. 15E: Independent binding of the 2-in-1 DARPins to target TargetA (plot 1) or scCD3εγ (plot 2) by ELISA. Parent DAPRins no 3 and 4 were used as positive controls. 2-in-1 DARPins and parent DARPins were tested at different concentrations as indicated.

FIG. 16: 2D paratope representations of 2-in-1 DARPin 21 (SEQ ID NO: 90), 2-in-1 DARPin 22 (SEQ ID NO: 91), 2-in-1 DARPin 23 (SEQ ID NO: 92), 2-in-1 DARPin 24 (SEQ ID NO: 93) and 2-in-1 DARPin 25 (SEQ ID NO: 94) as well as parent DARPins Nos 1, 2, 3, and 4. Black or white cells indicate the origin of each residue. Residues represented in bold are consensus residues. Residues represented in italic are mutated residues as compared to the parent sequences and are therefore neither directly originating from the first or second parent.

### DETAILED DESCRIPTION OF THE INVENTION

As disclosed and exemplified herein, the disclosure provides in a first aspect a method of producing designed repeat domains, preferably designed ankyrin repeat domains, that have binding specificity for two targets and wherein binding of said designed repeat domains to the two targets is mutually exclusive.

Designed ankyrin repeat domains are structural units of designed ankyrin repeat proteins. Designed repeat protein libraries, including designed ankyrin repeat protein libraries (WO2002/020565; Binz et al., Nat. Biotechnol. 22, 575-582, 2004; Stumpp et al., Drug Discov. Today 13, 695-701, 2008), can be used for the selection of target-specific designed repeat domains that bind to their target with high affinity. Such target-specific designed repeat domains in turn can be used as valuable components of recombinant binding proteins for the treatment of diseases.

Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain, or may comprise a combination of two, three, four, five or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two, three, four, five or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

The binding specificity of a designed ankyrin repeat domain is dictated by the nature of the repeat modules comprised in each repeat domain. Both internal repeat modules and terminal capping modules (C-cap or N-cap modules) can contribute to said specificity. Unexpectedly, the method of the present invention allows to produce single designed repeat domains (or mono-domains) having mutually exclusive binding specificity for two targets by combining repeat modules of two parental repeat domains. Said binding specificity for each of the first and second targets is therefore inherited from the binding specificity to the respective targets of the parental repeat domains. Such designed repeat domains of the invention maintain advantageous properties of conventional designed repeat domains, including binding characteristics. Designed repeat domains of the invention can be used to further broaden the applications of repeat proteins, for example as control mechanisms such as protein switches.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities of ingredients or reaction conditions used herein should be understood as modified in all instances by the term “about” as that term would be interpreted by the person skilled in the relevant art. The term “about” as used herein is equivalent to ±10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The term “nucleic acid” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid may either be present in isolated form or be comprised in recombinant nucleic acid molecules or vectors.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

The term “target” refers to an individual molecule such as a nucleic acid, a polypeptide or protein, a carbohydrate, or any other naturally or non-naturally occurring molecule, including any part of such individual molecule, or complexes of two or more of such molecules. The target may be a whole cell or a tissue sample, or it may be any non-natural molecule or moiety. Preferably, the target is a naturally occurring or nonnatural polypeptide or a polypeptide containing chemical modifications, for example modified by natural or non-natural phosphorylation, acetylation, or methylation.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Optionally, a repeat domain also comprises an N-terminal and/or a C-terminal capping module. For clarity, a capping module can be a repeat module, and as such can contribute to the two or more consecutive repeat modules of a repeat domain. Such repeat domains, repeat modules, and capping modules, sequence motives, as well as structural homology and sequence homology are well known to the practitioner in the art from examples of ankyrin repeat domains (Binz et al., J. Mol. Biol. 332, 489-503, 2003; Binz et al., 2004, loc. cit.; WO2002/020565; WO2012/069655), leucine-rich repeat domains (WO2002/020565), tetratricopeptide repeat domains (Main, E. R., Xiong, Y., Cocco, M. J., D'Andrea, L., Regan, L., Structure 11 (5), 497-508, 2003), and armadillo repeat domains (WO2009/040338). It is further well known to the practitioner in the art that such repeat domains are different from proteins comprising repeated amino acid sequences, where every repeated amino acid sequence is able to form an individual domain (for example FN3 domains of Fibronectin).

The term “ankyrin repeat domain” refers to a repeat domain comprising two or more consecutive ankyrin repeat modules as structural units, wherein said ankyrin repeat modules have structural and sequence homology.

The term “part” as used in “part of a repeat domain” (or the like) includes both a portion of the repeat domain or the entire repeat domain. Such a portion can include one or more repeat modules comprised in such a repeat domain or any fragment or stretch of amino acids comprised in the sequence of such a repeat domain. Preferably, a part of a repeat domain as described herein comprises no less than 10 (more preferably no less than 20, even more preferably no less than 30) consecutive amino acids of the amino acid sequence of the repeat domain. More preferably, such a part comprises at least one entire repeat module of the repeat domain.

The term “designed” as used in designed repeat protein, designed repeat domain and the like refers to the property that such repeat proteins and repeat domains, respectively, are man-made and do not occur in nature. The binding domains of the instant invention are designed repeat domains. Preferably, a designed repeat domain of the invention is a designed ankyrin repeat domain.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, preferably the family of ankyrin repeat proteins. Furthermore, each repeat module comprised in a repeat domain may comprise a “repeat sequence motif” deduced from homologous repeat modules obtained from repeat domains selected on a target and having the same target specificity. A repeat module as used in the present invention encompasses internal repeat modules and capping modules such as N-terminal and C-terminal capping modules. An “internal repeat module” refers to a repeat module that is flanked by two repeat modules. In other words, an internal repeat module is N-terminally flanked by one repeat module and C-terminally flanked by another repeat module.

Accordingly, the term “ankyrin repeat module” refers to a repeat module, which is originally derived from the repeat units of naturally occurring ankyrin repeat proteins. Ankyrin repeat proteins are well known to the person skilled in the art. Designed ankyrin repeat proteins have been described previously; see, e.g., International Patent Publication Nos. WO2002/020565, WO2010/060748, WO2011/135067, WO2012/069654, WO2012/069655, WO2014/001442, WO2014/191574, WO2014/083208, WO2016/156596, and WO2018/054971, all of which are incorporated by reference in their entireties. Typically, an ankyrin repeat module comprises about 31 to 33 amino acid residues that form two alpha helices, separated by loops.

Repeat modules may comprise positions with amino acid residues which have not been randomized in a library for the purpose of selecting target-specific repeat domains (“non-randomized positions” or “fixed positions” used interchangeably herein) and positions with amino acid residues which have been randomized in the library for the purpose of selecting target-specific repeat domains (“randomized positions”). Non-randomized positions comprise framework residues and may also comprise target interaction residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline.

The term “paratope” or “binding surface” refers to the region of a repeat domain which provides binding specificity for one or more target(s). A paratope as used herein may comprise target interaction residues and framework residues. The positions of paratope residues are defined as follows:
in internal repeat modules, positions corresponding to positions 3, 4, 6, 11, 14 and/or 15 of SEQ ID NO: 39 comprise target interaction residues, and a position corresponding to position 10 of SEQ ID NO: 39 comprises a framework residue; in N-terminal capping modules, positions corresponding to positions 4, 5, 8, 11 and/or 12 of SEQ ID NO: 36 comprise target interaction residues, and a position corresponding to position 7 of SEQ ID NO: 36 comprises a framework residue; in C-terminal capping modules, positions corresponding to positions 3, 4, 6, 14 and/or 15 of SEQ ID NO: 38 comprise target interaction residues, and positions corresponding to positions 10 and 11 of SEQ ID NO: 38 comprise framework residues.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “target interaction residues” refers to amino acid residues of a repeat module, which contribute to the direct interaction with a target. Such contribution of a residue can be tested, e.g., in a binding assay, for example in a mutagenesis study performed to identify residues required, sufficient, and/or necessary for a repeat domain to bind a target with its original binding affinity or quantity (i.e. its binding affinity or quantity in the absence of any mutations). In the context of the present invention, if a loss of at least 10% binding interaction between a repeat domain and a target results from a residue mutation within said domain, as compared to the binding interaction between the non-mutated repeat domain and the target, said residue is considered to contribute to the interaction with said target. Target interaction residues can also be determined by structural analyses of a repeat domain bound to a target.

The terms “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “binding specificity”, “has binding specificity for a target”, “specifically binding to a target”, “binding to a target with high specificity”, “specific for a target” or “target specificity” and the like means that a binding protein or binding domain binds to a target with a lower dissociation constant (i.e. it binds with higher affinity) than it binds to an unrelated protein such as the E. coli maltose binding protein (MBP). Preferably, the dissociation constant (“KD”) for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of KD values are preferably made with standardized solutions of protein and a standardized buffer, such as PBS.

Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {⇌}_{{k}_{off}}^{{k}_{o n}} [ BT ]$$

kₒₙ is a second-order rate constant of the binding reaction, with the unit M⁻¹s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015-5-5; 15 (5): 10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). A K_{D} value may be measured using OCTET® technology (Octet QKe system, ForteBio). Alternatively, or in addition, a KinExA® (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred. Most preferably, the K_{D} values are determined in PBS and by SPR. A typical and preferred determination of K_{D} of designed repeat domains binding a target by SPR analysis is described in Example 2.

The term “PBS” means a phosphate buffered water solution containing 137 mM NaCl, 10 mM phosphate and 2.7 mM KCl and having a pH of 7.4.

The term “dual binding specificity” or “dual-specific” refers to the property that designed repeat domains of the invention have binding specificity for two targets, wherein the two targets may be two epitopes on a single target molecule or two epitopes where each one of said two epitopes is located on a different target molecule. The epitopes are preferably different from each other.

The term “covalently combining” or “covalent combination” as used in covalently combining repeat modules includes “covalently connecting” and “covalently merging” said repeat modules. Covalently connecting two repeat modules refers to directly connecting said repeat modules through a chemical bond such as a peptide bond. Covalently connecting encompasses a connection by a direct peptide bond or by a linker between said repeat modules. Such a linker may be a non-peptidic chemical linker or a peptide linker, wherein said peptide linker may be any suitable peptide linker comprising 1, 2, 3, 4, 5, 6, 7, 8, 9, 10, 11, 12, 13, 14, 15, 16, 17, 18, 19, 20, 21, 22, 23, 24 or 25 amino acids. Alternatively said peptide linker is an entire non-binding repeat module. Examples of non-binding repeat modules are described herein. Covalently merging two (parent) repeat modules refers to the process of creating a single hybrid repeat module from two parent repeat modules. Such a hybrid repeat module comprises sequence elements from both parent repeat modules.

The term “merged module” or “mixed module” refers to a repeat module, preferably an ankyrin repeat module, which can be obtained by covalently merging two parent repeat modules and comprises at least one target interaction residue of the first parent repeat module and at least one target interaction residue of the second parent repeat module.

The term “mutual exclusivity” or “mutually exclusive” as used in mutually exclusive binding refers to the property of designed repeat domains of the invention to be able to bind a first target and a second target only in a substantially non-simultaneous manner. In such a mutually exclusive binding, binding of said first target to the repeat domain influences binding of said second target to the repeat domain (and/or vice versa) such that simultaneous binding of said first and second targets is substantially prevented. Underlying causes for this effect include steric hindrance and/or an overlap of paratopes. In the context of the present invention, a binding interaction between a designed repeat domain of the invention and its first and second targets is considered to be mutually exclusive if, when bound to one of said first and second targets, said repeat domain cannot bind more than 1%, more than 2%, more than 3%, more than 4%, more than 5%, more than 10%, more than 15%, more than 20%, more than 25%, more than 30%, more than 35%, more than 40%, more than 45% or more than 50% of the molar equivalent of the other target. The extent of such mutual exclusivity can be measured by competitive binding assays in which binding of the first target to the repeat domain is compared to the binding of the second target to the repeat domain. Such a competitive binding assay may be adapted from standard competitive binding assays used in the field of antibodies, as known by the person skilled in the art.

The term “therapeutic agent” as used herein refers to a drug, molecule, nucleic acid, protein, composition or other substance that provides a therapeutic effect. The terms “therapeutic agent” and “drug” are used interchangeably.

Throughout the present disclosure, the order or orientation with regard to the “first” and “second” entity, as used for instance in first and second targets, first and second repeat domains, first and second binding specificities, etc. is not meant to specify one specific order or orientation and encompasses the alternative, unless specifically defined. In other words, any reference to such a first and second entity also encompasses the alternative order where the first entity corresponds to the second one and the second entity corresponds to the first one, unless specifically defined otherwise.

### Production Method According to the Invention

In one aspect, the invention provides a method of producing a designed repeat domain, the method comprising the steps of:
(i) providing a first repeat domain and a second repeat domain, wherein said first repeat domain has binding specificity for a first target and said second repeat domain has binding specificity for a second target; and
(ii) joining part of said first repeat domain and part of said second repeat domain to form a designed repeat domain with dual binding specificity, by covalently combining at least one repeat module of said first repeat domain and at least one repeat module of said second repeat domain;
   wherein said designed repeat domain has binding specificity for said first target and binding specificity for said second target, and wherein binding of said designed repeat domain to said first and second targets is mutually exclusive.

DARPins (designed ankyrin repeat proteins) are repeat proteins comprising an ankyrin repeat domain, which typically comprises one or more internal repeat modules that are identical or similar except for their randomized positions and that are flanked by an N-terminal and a C-terminal capping repeat module (see, e.g., Binz et al. 2003). As used herein, the term “repeat module” encompasses internal repeat modules and terminal repeat modules (C-cap and N-cap modules). 27 of the 33 amino acid positions of typical internal repeat modules are highly conserved, whereas the other 6 amino acid positions are less conserved and to the most part responsible for the specific interaction of the ankyrin repeat domain with its target (Binz et al. 2003). The paratope of the ankyrin repeat domain is formed by the continuous surface formed largely by these variable positions of the internal repeat modules and sometimes also the capping repeat modules.

The inventors of the present invention have surprisingly discovered that single designed repeat domains (i.e. mono-domains) with dual and mutually exclusive binding specificity can be generated by combining amino acid sequence elements, such as repeat modules, from two parental repeat domains, wherein the parental repeat domains are preferably both ankyrin repeat domains and the generated designed repeat domain with dual and mutually exclusive binding specificity is thus also an ankyrin repeat domain.

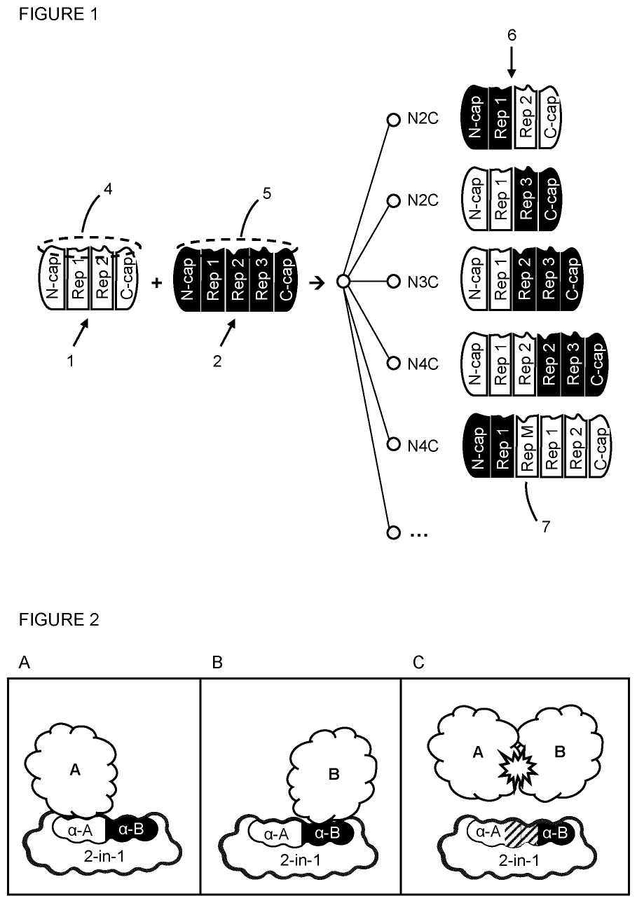

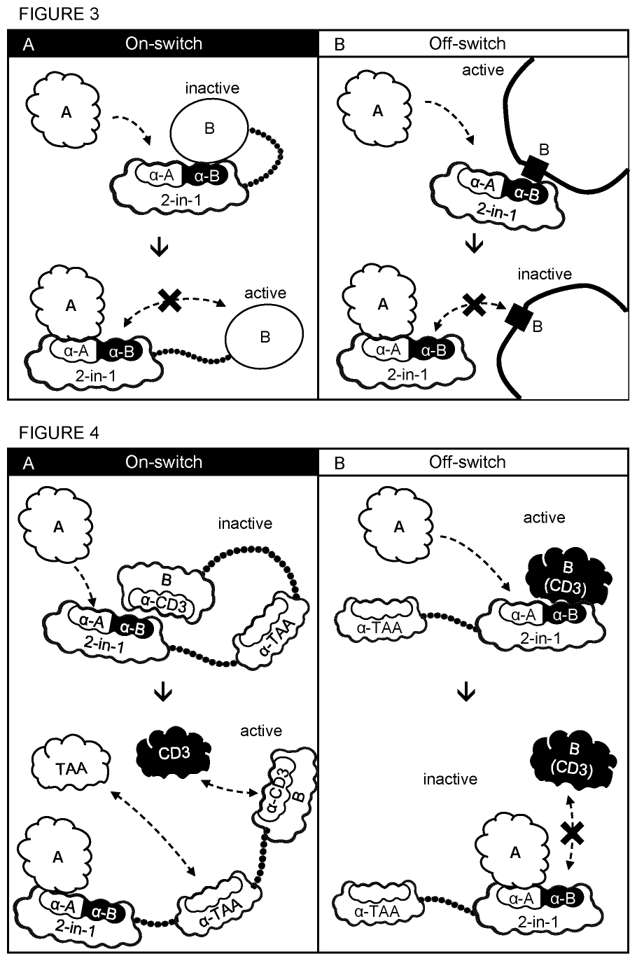

The basic concept of the production method according to the present invention is illustrated in FIG. 1. As can be seen from FIG. 1, a portion of a first (parent) repeat domain (1) having binding specificity for a first target (target A), and a portion of a second (parent) repeat domain (2) having binding specificity for a second target (target B), can be covalently combined to form a new designed repeat domain. The respective paratopes or binding surfaces of the parent repeat domains are shown ((4) and (5)). Such newly formed designed repeat domains are also referred herein as “2-in-1 DARPin” or “SwitchDARPin” (6). Such a designed repeat domain can bind the two targets A and B in a mutually exclusive manner, i.e. either target A or target B. Additionally, one or several internal repeat modules in the designed repeat domain can be mixed (or merged) repeat modules (7). A repeat module is called a mixed repeat module if its paratope comprises at least one target interaction residue from the corresponding module in the first parent repeat protein and at least one target interaction residue from the corresponding module in the second parent repeat protein. The mutually exclusive binding property of the designed repeat domain of the invention is illustrated in FIG. 2. Due to the close proximity or overlap of the two paratopes of the designed repeat domain of the invention, said designed repeat domain is able to bind target A (FIG. 2A) or target B (FIG. 2B) individually, but not both targets at the same time (FIG. 2C), as the simultaneous binding of both targets A and B is sterically hindered. This mutually exclusive binding feature in consequence enables the design of on- or off-switches for conditional activation or de-activation of drug molecules or other target molecules (such as, e.g., a cell surface receptor), as illustrated in FIGS. 3 and 4. This mutually exclusive binding feature also distinguishes the designed repeat domain of the invention from other ankyrin repeat protein formats (Mohan, K. et al, 2019, Science, 364 (6442); WO 2020/190852 A2).

Covalent combination as used in the present invention includes covalently connecting or covalently merging parental repeat modules. This distinction depends on the nature and position of target interaction residues comprised in the repeat module(s) resulting from the covalent combination as compared to the target interaction residues comprised in the parental modules. Covalently connecting a first and a second parental repeat module means to connect both parental repeat modules by a chemical bond such as a peptide bond. Covalently connecting alternatively refers to connecting said first and second repeat modules by a linker. Covalently merging a first and a second parental repeat module results in a single merged (or mixed) repeat module which comprises at least one target interaction residue from the first parental repeat module and at least one target interaction residue from the second parental repeat module. Accordingly, k merged repeat modules may be obtained from covalently merging 2 k parental repeat modules. An effect of such covalent combinations is that the resulting designed repeat domain can bind both targets only in a mutually exclusive way due to steric hindrance between the two targets. In the covalent merging case, the presence of any merged repeat module may further enhance the mutually exclusive binding effect of the designed repeat domain due to the fact that such merged modules can either bind to one or the other target at a given time.

Thus, in one embodiment, said covalently combining at least one repeat module of said first repeat domain and at least one repeat module of said second repeat domain comprises covalently connecting said at least one repeat module of said first repeat domain and said at least one repeat module of said second repeat domain. Covalently connecting such repeat modules can be achieved by methods well known in the art and may involve any type of covalent bond. In a preferred embodiment, said covalently connecting involves a peptide bond. In a preferred embodiment, said covalently combining comprises covalently connecting one repeat module of said first repeat domain and one repeat module of said second repeat domain. In another embodiment, said covalently combining comprises covalently connecting one repeat module of said first repeat domain and one repeat module of said second repeat domain by a linker. In one embodiment, said linker is a chemical non-peptidic linker. In another embodiment, said linker is a peptide linker, wherein preferably said peptide linker comprises 1, 2, 3, 4, 5, 6, 7, 8, 9, 10, 11, 12, 13, 14, 15, 16, 17, 18, 19, 20, 21, 22, 23, 24 or 25 amino acids. In another embodiment, said linker is a non-binding repeat module. An example of such a non-binding repeat module is provided by SEQ ID NO: 89.

In one embodiment, said covalently combining at least one repeat module of said first repeat domain and at least one repeat module of said second repeat domain comprises covalently merging said at least one repeat module of said first repeat domain and said at least one repeat module of said second repeat domain. In another embodiment, one repeat module of said first repeat domain may be covalently merged to one repeat module of said second repeat domain. In another embodiment, two repeat modules of said first repeat domain may be covalently merged to two repeat modules of said second repeat domain. In another embodiment, three repeat modules of said first repeat domain may be covalently merged to three repeat modules of said second repeat domain.

In some embodiments, said first and/or said second repeat domain(s) may independently comprise at least 1 repeat module, at least 2 repeat modules, at least 3 repeat modules, at least 4 repeat modules, at least 5 repeat modules or at least 6 repeat modules. In some embodiments, said first and/or second repeat domain(s) may independently comprise between 1 and 6 repeat modules, between 2 and 6 repeat modules, between 3 and 6 repeat modules, between 4 and 6 repeat modules or between 5 and 6 repeat modules. In some embodiments, said first and/or second repeat domain(s) may independently comprise an N-terminal capping module and/or a C-terminal capping module. In some embodiments, said first and/or second repeat domain(s) may independently comprise an N-terminal capping module and/or a C-terminal capping module and at least 1 internal repeat module, at least 2 internal repeat modules, at least 3 internal repeat modules or at least 4 internal repeat modules. In some embodiments, said first and/or second repeat domain(s) may independently comprise an N-terminal capping module and/or a C-terminal capping module and between 1 and 4 internal repeat modules, between 1 and 3 internal repeat modules or between 1 and 2 internal repeat modules.

In some preferred embodiments, said first repeat domain comprises between 3 and 5 repeat modules and said second repeat domain comprises between 3 and 5 repeat modules. In some more preferred embodiments, said first repeat domain comprising between 3 and 5 repeat modules comprises from N- to C-terminus: a N-terminal capping module, between 1 and 3 internal repeat modules and a C-terminal capping module, and said second repeat domain comprising between 3 and 5 repeat modules comprises from N- to C-terminus: a N-terminal capping module, between 1 and 3 internal repeat modules and a C-terminal capping module.

In some preferred embodiments, each of said first and second repeat domains comprises 4 repeat modules. In some more preferred embodiments, each of said first and second repeat domains comprising 4 repeat modules comprises from N- to C-terminus: a N-terminal capping module, two internal repeat modules and a C-terminal capping module.

In some preferred embodiments, each of said first and second repeat domains comprises 5 repeat modules. In some more preferred embodiments, each of said first and second repeat domains comprising 5 repeat modules comprises from N- to C-terminus: a N-terminal capping module, three internal repeat modules and a C-terminal capping module.

In some preferred embodiments, said first repeat domain comprises 4 repeat modules and said second repeat domain comprises 5 repeat modules. In some more preferred embodiments, said first repeat domain comprising 4 repeat modules comprises from N- to C-terminus: a N-terminal capping module, two internal repeat modules and a C-terminal capping module, and said second repeat domain comprising 5 repeat modules comprises from N- to C-terminus: a N-terminal capping module, three internal repeat modules and a C-terminal capping module.

In one embodiment, said designed repeat domain comprises an N-terminal capping module and/or a C-terminal capping module, and at least one internal repeat module. In one embodiment, said designed repeat domain comprises an N-terminal capping module and/or a C-terminal capping module, and one internal repeat module. In one embodiment, said designed repeat domain comprises an N-terminal capping module and/or a C-terminal capping module, and two internal repeat modules. In one embodiment, said designed repeat domain comprises an N-terminal capping module and/or a C-terminal capping module, and three internal repeat modules. In one embodiment, said designed repeat domain comprises an N-terminal capping module and/or a C-terminal capping module, and four internal repeat modules. In one embodiment, said designed repeat domain comprises an N-terminal capping module and/or a C-terminal capping module, and five internal repeat modules. In any of the above embodiments, the “and” conjunction is preferred. Accordingly, in some embodiments, said designed repeat domain comprises an N-terminal capping module and a C-terminal capping module, and at least one internal repeat module. In one embodiment, said designed repeat domain comprises an N-terminal capping module and a C-terminal capping module, and one internal repeat module. In one embodiment, said designed repeat domain comprises an N-terminal capping module and a C-terminal capping module, and two internal repeat modules. In one embodiment, said designed repeat domain comprises an N-terminal capping module and a C-terminal capping module, and three internal repeat modules. In one embodiment, said designed repeat domain comprises an N-terminal capping module and a C-terminal capping module, and four internal repeat modules. In one embodiment, said designed repeat domain comprises an N-terminal capping module and a C-terminal capping module, and five internal repeat modules.

In one embodiment, said N-terminal capping module is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the N-terminal capping module comprised in said first repeat domain.

In one embodiment, said C-terminal capping module is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the C-terminal capping module comprised in said second repeat domain.

In one embodiment, said N-terminal capping module is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the N-terminal capping module comprised in said first repeat domain and said C-terminal capping module is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the C-terminal capping module comprised in said second repeat domain.

Thus, in a particular embodiment, said N-terminal capping module is at least 90% identical in amino acid sequence to the N-terminal capping module comprised in said first repeat domain and said C-terminal capping module is at least 90% identical in amino acid sequence to the C-terminal capping module comprised in said second repeat domain. In some embodiments, the number of repeat modules comprised in said designed repeat domain corresponds to the sum of the repeat modules comprised in said part of the first repeat domain and the repeat modules comprised in said part of the second repeat domain. In some embodiments, the number of repeat modules comprised in said designed repeat domain corresponds to n-m, where n is the sum of the repeat modules comprised in said part of the first repeat domain and the repeat modules comprised in said part of the second repeat domain, and m is the number of merged modules in said designed repeat domain.

In some embodiments, said part of the first repeat domain comprises a chain of repeat modules comprising x repeat modules and/or said part of the second repeat domain comprises a chain of repeat modules comprising y repeat modules, wherein x is an integer selected in the range from 1 to the total number of repeat modules comprised in said first repeat domain and y is an integer selected in the range from 1 to the total number of repeat modules comprised in said second repeat domain.

In some embodiments, said part of the first repeat domain may be selected from the group consisting of a chain of 2 repeat modules, a chain of 3 repeat modules, a chain of 4 repeat modules, a chain of 5 repeat modules and a chain of 6 repeat modules comprised in said first repeat domain and/or said part of the second repeat domain may be selected from the group consisting of a chain of 2 repeat modules, a chain of 3 repeat modules, a chain of 4 repeat modules, a chain of 5 repeat modules and a chain of 6 repeat modules comprised in said second repeat domain. In some embodiments, said chain is a consecutive chain of repeat modules as compared to the order of repeat modules comprised in the corresponding first or second repeat domain. In some embodiments, said part of the first repeat domain comprises the entire first repeat domain and/or said part of the second repeat domain comprises the entire second repeat domain. In the above embodiments, the length or size of said part of the first repeat domain may be independent of the length or size of said part of the second repeat domain.

In one embodiment, said part of said first repeat domain comprises the N-terminal capping module and at least one internal repeat module of said first repeat domain and said part of said second repeat domain comprises the C-terminal capping module and at least one internal repeat module of said second repeat domain. In one embodiment, said part of said first repeat domain comprises the C-terminal capping module and at least one internal repeat module of said first repeat domain and said part of said second repeat domain comprises the N-terminal capping module and at least one internal repeat module of said second repeat domain. In an alternative embodiment, said part of said first repeat domain comprises at least one internal repeat module of said first repeat domain and said part of said second repeat domain comprises the N-terminal capping module, the C-terminal capping module and at least one internal repeat module of said second repeat domain.

In some embodiments, said part of the first repeat domain may represent between 1% and 5%, between 1% and 10%, between 1% and 15%, between 1% and 20%, between 1% and 25%, between 1% and 30%, between 1% and 35%, between 1% and 40%, between 1% and 45%, between 1% and 50%, between 1% and 55%, between 1% and 60%, between 1% and 65%, between 1% and 70%, between 1% and 75%, between 1% and 80%, between 1% and 85%, between 1% and 90%, between 1% and 95% or between 1% and 100% of the amino acid sequence of said first repeat domain and/or said part of the second repeat domain may represent between 1% and 5%, between 1% and 10%, between 1% and 15%, between 1% and 20%, between 1% and 25%, between 1% and 30%, between 1% and 35%, between 1% and 40%, between 1% and 45%, between 1% and 50%, between 1% and 55%, between 1% and 60%, between 1% and 65%, between 1% and 70%, between 1% and 75%, between 1% and 80%, between 1% and 85%, between 1% and 90%, between 1% and 95% or between 1% and 100% of the amino acid sequence of said second repeat domain.

In some embodiments, said part of the first repeat domain may represent between 1% and 100%, between 5% and 100%, between 10% and 100%, between 15% and 100%, between 20% and 100%, between 25% and 100%, between 30% and 100%, between 35% and 100%, between 40% and 100%, between 45% and 100%, between 50% and 100%, between 55% and 100%, between 60% and 100%, between 65% and 100%, between 70% and 100%, between 75% and 100%, between 80% and 100%, between 85% and 100%, between 90% and 100% or between 95% and 100% of the amino acid sequence of said first repeat domain and/or said part of the second repeat domain may represent between 1% and 100%, between 5% and 100%, between 10% and 100%, between 15% and 100%, between 20% and 100%, between 25% and 100%, between 30% and 100%, between 35% and 100%, between 40% and 100%, between 45% and 100%, between 50% and 100%, between 55% and 100%, between 60% and 100%, between 65% and 100%, between 70% and 100%, between 75% and 100%, between 80% and 100%, between 85% and 100%, between 90% and 100% or between 95% and 100% of the amino acid sequence of said second repeat domain.

In some embodiments, said part of the first repeat domain may represent about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% of the amino acid sequence of said first repeat domain and/or said part of the second repeat domain may represent about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% of the amino acid sequence of said second repeat domain. In the above embodiments, the length or size of said part of the first repeat domain may be independent of the length or size of said part of the second repeat domain. In some embodiments, said parts are consecutive amino acid sequences as compared to the corresponding amino acid sequence of the first and second repeat domains.

In some embodiments, any such part of the first repeat domain is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the corresponding amino acid sequence of said first repeat domain and/or any such part of the second repeat domain is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the corresponding amino acid sequence of said second repeat domain.

In one embodiment, an N-terminal fragment of said first repeat domain may be covalently combined with a C-terminal fragment of said second repeat domain. In another embodiment, a C-terminal fragment of said first repeat domain may be covalently combined with an N-terminal fragment of said second repeat domain.

In some embodiments, such fragments of the first repeat domain may represent an amino acid sequence corresponding to between 1% and 5%, between 1% and 10%, between 1% and 15%, between 1% and 20%, between 1% and 25%, between 1% and 30%, between 1% and 35%, between 1% and 40%, between 1% and 45%, between 1% and 50%, between 1% and 55%, between 1% and 60%, between 1% and 65%, between 1% and 70%, between 1% and 75%, between 1% and 80%, between 1% and 85%, between 1% and 90%, between 1% and 95% or between 1% and 100% of consecutive amino acids comprised in the amino acid sequence of said first repeat domain and/or such fragments of the second repeat domain may represent an amino acid sequence corresponding to between 1% and 5%, between 1% and 10%, between 1% and 15%, between 1% and 20%, between 1% and 25%, between 1% and 30%, between 1% and 35%, between 1% and 40%, between 1% and 45%, between 1% and 50%, between 1% and 55%, between 1% and 60%, between 1% and 65%, between 1% and 70%, between 1% and 75%, between 1% and 80%, between 1% and 85%, between 1% and 90%, between 1% and 95% or between 1% and 100% of consecutive amino acids comprised in the amino acid sequence of said second repeat domain.

In some embodiments, such fragments of the first repeat domain may represent a consecutive amino acid stretch corresponding to between 1% and 100%, between 5% and 100%, between 10% and 100%, between 15% and 100%, between 20% and 100%, between 25% and 100%, between 30% and 100%, between 35% and 100%, between 40% and 100%, between 45% and 100%, between 50% and 100%, between 55% and 100%, between 60% and 100%, between 65% and 100%, between 70% and 100%, between 75% and 100%, between 80% and 100%, between 85% and 100%, between 90% and 100% or between 95% and 100% of consecutive amino acids comprised in the amino acid sequence of said first repeat domain and/or such fragments of the second repeat domain may represent an amino acid sequence corresponding to between 1% and 100%, between 5% and 100%, between 10% and 100%, between 15% and 100%, between 20% and 100%, between 25% and 100%, between 30% and 100%, between 35% and 100%, between 40% and 100%, between 45% and 100%, between 50% and 100%, between 55% and 100%, between 60% and 100%, between 65% and 100%, between 70% and 100%, between 75% and 100%, between 80% and 100%, between 85% and 100%, between 90% and 100% or between 95% and 100% of consecutive amino acids comprised in the amino acid sequence of said second repeat domain.

In some embodiments, such fragments of the first repeat domain may represent an amino acid sequence corresponding to about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% of consecutive amino acids comprised in the amino acid sequence of said first repeat domain and/or such fragments of the second repeat domain may represent an amino acid sequence corresponding to about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% of consecutive amino acids comprised in the amino acid sequence of said second repeat domain. In the above embodiments, the length or size of said fragment of the first repeat domain may be independent of the length or size of said fragment of the second repeat domain.

In a preferred embodiment, a fragment of a repeat domain corresponds to an amino acid sequence starting from one terminus (either N-terminus or C-terminus) of said repeat domain (such as the first or second repeat domain in the embodiments above). As used herein, covalently combining a N-terminal fragment of a repeat domain with the C-terminal fragment of another repeat domain includes covalently connecting or covalently merging both fragments.

In some embodiments, any such fragment of the first repeat domain is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the corresponding amino acid sequence of said first repeat domain and/or any such fragment of the second repeat domain is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical in amino acid sequence to the corresponding amino acid sequence of said second repeat domain.

In one embodiment, the method of the invention further comprises step (i)(2) after step (i), wherein step (i)(2) comprises evaluating said first and/or said second repeat domains for their compatibility with being covalently combined with another repeat domain at the N-terminal side and/or at the C-terminal side. The extent of said evaluation may vary depending on the structural properties of said first and second repeat domains and their respective targets. In some embodiments, said evaluation comprises at least determining if the N-terminal and/or C-terminal capping modules of said first repeat domain contribute to the binding of said first repeat domain to said first target, and/or determining if the N-terminal and/or C-terminal capping modules of said second repeat domain contribute to the binding of said second repeat domain to said second target. In some embodiments, said evaluation comprises determining if the N-terminal and/or C-terminal capping modules and the internal repeat module(s) adjacent to said N-terminal and/or C-terminal terminal capping modules of said first repeat domain contribute to the binding of said first repeat domain to said first target, and/or determining if the N-terminal and/or C-terminal capping modules and the internal repeat module(s) adjacent to said N-terminal and/or C-terminal terminal capping modules of said second repeat domain contribute to the binding of said second repeat domain to said second target.

An exemplary way to perform this binding contribution determination is to create variants of said first and/or second repeat domains and subsequently perform binding assays to determine whether or not the modifications made in the variants impact their binding properties, such as resulting in a loss of binding or in the maintenance of binding. Based on the outcome of the variants' evaluation, covalent combination compatibility of said first and/or second repeat domain can be inferred. Assays used in said evaluation or binding contribution determination include an ELISA binding assay or any other suitable test assay known by a skilled person in the art. When an ELISA binding assay is used, an EC50 value of about 50 nm, about 100 nm, about 150 nm, about 200 nm, about 250 nm, about 300 nm, about 350 nm or about 400 nm may be used as threshold to classify whether a loss of binding occurred or whether binding was maintained. Preferably, an EC50 value of 300 nm is used as such a threshold.

Such a determination of covalent combination compatibility is described in Example 9.1 (the method used is also referred to as profiling method).

Accordingly, in one embodiment, the invention provides a method of producing a designed repeat domain, the method comprising the steps of:
(i) providing a first repeat domain and a second repeat domain, wherein said first repeat domain has binding specificity for a first target and said second repeat domain has binding specificity for a second target,
(i)(2) determining whether said first repeat domain and/or said second repeat domain is compatible with being covalently combined with another repeat domain at the N-terminal side and/or the C-terminal side; and
(ii) joining part of said first repeat domain and part of said second repeat domain to form a designed repeat domain with dual binding specificity, by covalently combining at least one repeat module of said first repeat domain and at least one repeat module of said second repeat domain;
   wherein said designed repeat domain has binding specificity for said first target and binding specificity for said second target, and wherein binding of said designed repeat domain to said first and second targets is mutually exclusive.

In one embodiment, step (ii) of the method of the invention further comprises removing the C-terminal capping module of said first repeat domain and removing the N-terminal capping module of said second repeat domain, such that said C-terminal capping module of said first repeat domain and said N-terminal capping module of said second repeat domain are not comprised in said designed repeat domain.

In one embodiment, step (ii) of the method of the invention further comprises removing the C-terminal capping module of said first repeat domain and/or removing the N-terminal capping module of said second repeat domain, such that said C-terminal capping module of said first repeat domain and/or said N-terminal capping module of said second repeat domain are not comprised in said designed repeat domain.

In one embodiment, step (ii) of the method of the invention further comprises removing the N-terminal capping module of said first repeat domain and/or removing the C-terminal capping module of said second repeat domain, such that said N-terminal capping module of said first repeat domain and/or said C-terminal capping module of said second repeat domain are not comprised in said designed repeat domain.

In one embodiment, step (ii) of the method of the invention further comprises removing one or more repeat modules from the C-terminal end of said first repeat domain and removing one or more repeat modules from the N-terminal end of said second repeat domain, such that said one or more repeat modules from the C-terminal end of said first repeat domain and said one or more repeat modules from the N-terminal end of said second repeat domain are not comprised in said designed repeat domain. In one embodiment, step (ii) of said method further comprises removing one or more repeat modules from the N-terminal end of said first repeat domain and removing one or more repeat modules from the C-terminal end of said second repeat domain, such that said one or more repeat modules from the N-terminal end of said first repeat domain and said one or more repeat modules from the C-terminal end of said second repeat domain are not comprised in said designed repeat domain.

In one embodiment, step (ii) of the method of the invention further comprises removing one or more repeat modules from the C-terminal end of said first repeat domain and/or removing one or more repeat modules from the N-terminal end of said second repeat domain, such that said one or more repeat modules from the C-terminal end of said first repeat domain and/or said one or more repeat modules from the N-terminal end of said second repeat domain are not comprised in said designed repeat domain. In one embodiment, step (ii) of said method further comprises removing one or more repeat modules from the N-terminal end of said first repeat domain and/or removing one or more repeat modules from the C-terminal end of said second repeat domain, such that said one or more repeat modules from the N-terminal end of said first repeat domain and/or said one or more repeat modules from the C-terminal end of said second repeat domain are not comprised in said designed repeat domain.

In one embodiment, the method of the invention further comprises step (ii)(2), wherein step (ii)(2) comprises determining whether binding of the designed repeat domain formed in step (ii) to said first and second targets is mutually exclusive. An exemplary determination of such mutually exclusive target binding of a designed repeat domain is described in Example 9.

Accordingly, in one embodiment, the invention provides a method of producing a designed repeat domain, the method comprising the steps of:
(i) providing a first repeat domain and a second repeat domain, wherein said first repeat domain has binding specificity for a first target and said second repeat domain has binding specificity for a second target,
(ii) joining part of said first repeat domain and part of said second repeat domain to form a designed repeat domain with dual binding specificity, by covalently combining at least one repeat module of said first repeat domain and at least one repeat module of said second repeat domain; and
(ii)(2) determining whether binding of said designed repeat domain formed in step (ii) to said first and second targets is mutually exclusive;
   wherein said designed repeat domain has binding specificity for said first target and binding specificity for said second target, and wherein binding of said designed repeat domain to said first and second targets is mutually exclusive.

In another embodiment, the invention provides a method of producing a designed repeat domain, the method comprising the steps of:
(i) providing a first repeat domain and a second repeat domain, wherein said first repeat domain has binding specificity for a first target and said second repeat domain has binding specificity for a second target,
(i)(2) determining whether said first repeat domain and/or said second repeat domain is compatible with being covalently combined with another repeat domain at the N-terminal side and/or the C-terminal side;
(ii) joining part of said first repeat domain and part of said second repeat domain to form a designed repeat domain with dual binding specificity, by covalently combining at least one repeat module of said first repeat domain and at least one repeat module of said second repeat domain; and
(ii)(2) determining whether binding of said designed repeat domain formed in step (ii) to said first and second targets is mutually exclusive;
   wherein said designed repeat domain has binding specificity for said first target and binding specificity for said second target, and wherein binding of said designed repeat domain to said first and second targets is mutually exclusive.

In one embodiment, the method of the invention further comprises a step (ib) of comparing target interaction residues of the repeat modules comprised in said first repeat domain and target interaction residues of the repeat modules comprised in said second repeat domain, and selecting said at least one repeat module of the first repeat domain and said at least one repeat module of the second repeat domain for covalent combination.

Accordingly, in one embodiment, the invention provides a method of producing a designed repeat domain, the method comprising the steps of:
(i) providing a first repeat domain and a second repeat domain, wherein said first repeat domain has binding specificity for a first target and said second repeat domain has binding specificity for a second target;
(ib) comparing target interaction residues in said first repeat domain and target interaction residues in said second repeat domain and selecting a part of said first repeat domain and a part of said second repeat domain; and
(ii) joining said part of the first repeat domain and said part of the second repeat domain to form a designed repeat domain with dual binding specificity, by covalently combining at least one repeat module of said first repeat domain and at least one repeat module of said second repeat domain;
   wherein said designed repeat domain has binding specificity for said first target and binding specificity for said second target, and wherein binding of said designed repeat domain to said first and second targets is mutually exclusive.

In some preferred embodiments, said first target and/or said second target is/are (a) molecule(s) having biological activity. Many molecules having biological activity are known to one of skill in the art. One example of molecules having biological activity are agonists or antagonists or modulators of cell surface receptors and the cell surface receptors themselves. As known by the skilled person in the art, a molecule having biological activity may, for example, function by interacting with a receptor on a cell surface, thereby stimulating, inhibiting, or modulating the activity of the cell surface receptor. Such activity of the cell surface receptor, in turn, often involves intracellular signalling pathways of cells carrying the receptor, wherein the signalling pathways comprise biologically active molecules, such as enzymes, e.g. protein kinases, and transcription factors. More generally, molecule(s) having biological activity may exert their biological activity by binding to one or more binding partners. The binding partner of such a molecule can be an extracellular, intracellular or transmembrane protein, such as, e.g., a soluble receptor, a membrane bound receptor, a ligand, a cytokine, an enzyme, binding protein or a structural protein. Accordingly, in one embodiment, said designed repeat domain, when bound to said first target or said second target, inhibits a biological activity of the bound target. In another embodiment, said designed repeat domain, when bound to said first target or said second target, activates a biological activity of the bound target. In another embodiment, said designed repeat domain, when bound to said first target or said second target, modulates a biological activity of the bound target.

In context of the present invention, preferred examples of a target molecule having biological activity are any synthetic or naturally occurring molecules, such as polypeptides or proteins, which are either applicable for use in the diagnosis, cure, mitigation, treatment or prevention of a disease, disorder, condition or infection, or are present in a living organism. In one embodiment, a target molecule having biological activity is a drug molecule. In one embodiment, said drug molecule is a T cell engager.

In one embodiment, binding of said designed repeat domain to one of said first and second targets sterically hinders binding of said designed repeat domain to the other of said first and second targets.

In some embodiments, the binding affinity of said designed repeat domain to said first target may be described in terms of dissociation constant (K_{D}) values. In one embodiment, said designed repeat domain binds to said first target with a dissociation constant (K_{D}) below 10⁻⁵ M. In certain embodiments, said designed repeat domain binds to said first target with a K_{D} of about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻¹¹ M to about 10⁻¹⁵ M, from about 10⁻¹² M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻¹¹ M to about 10⁻¹⁴ M, from about 10⁻¹² M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, from about 10⁻¹⁰ M to about 10⁻¹³ M, from about 10⁻¹¹ M to about 10⁻¹³ M, or from about 10⁻¹² M to about 10⁻¹³ M. In further embodiments, said designed repeat domain binds to said first target with a K_{D} value of, or less than: about 1000 nM, about 100 nM, about 50 nM, about 25 nM, about 10 nM, about 5 nM, about 2 nM, about 1 nM, about 900 pM, about 800 pM, about 700 pM, about 600 pM, about 500 pM, about 400 PM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In one exemplary embodiment, said designed repeat domain binds to said first target with a K_{D} value of less than or equal to about 1 nM. In another exemplary embodiment, said designed repeat domain binds to said first target with a K_{D} value of less than or equal to about 100 pM. In another exemplary embodiment, said designed repeat domain binds to said first target with a K_{D} value of less than or equal to about 10 pM. In yet another exemplary embodiment, said designed repeat domain binds to said first target with a K_{D} value of less than or equal to about 1 pM.

In some embodiments, the binding affinity of said designed repeat domain to said second target may be described in terms of dissociation constant (K_{D}) values. In one embodiment, said designed repeat domain binds to said second target with a dissociation constant (K_{D}) below 10⁻⁵ M. In certain embodiments, said designed repeat domain binds to said second target with a K_{D} of about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10.5 M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻¹¹ M to about 10⁻¹⁵ M, from about 10⁻¹² M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻¹¹ M to about 10⁻¹⁴ M, from about 10⁻¹² M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10.9 M to about 10⁻¹³ M, from about 10⁻¹⁰ M to about 10⁻¹³ M, from about 10⁻¹¹ M to about 10⁻¹³ M, or from about 10⁻¹² M to about 10⁻¹³ M. In further embodiments, said designed repeat domain binds to said second target with a K_{D} value of, or less than: about 1000 nM, about 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In one exemplary embodiment, said designed repeat domain binds to said second target with a K_{D} value of less than or equal to about 1 nM. In another exemplary embodiment, said designed repeat domain binds to said second target with a K_{D} value of less than or equal to about 100 pM. In another exemplary embodiment, said designed repeat domain binds to said second target with a K_{D} value of less than or equal to about 10 pM. In yet another exemplary embodiment, said designed repeat domain binds to said second target with a K_{D} value of less than or equal to about 1 pM.

In some embodiments, the binding affinity of said designed repeat domain to each of said first and second target may be described in terms of dissociation constant (K_{D}) values. In such embodiments, K_{D}1 represents the dissociation constant between said designed repeat domain and said first target and K_{D}2 represents the dissociation constant between said designed repeat domain and said second target. In exemplary embodiments, said designed repeat domain binds to each of said first and second target with K_{D}1 and K_{D}2 being independently about 10⁻⁵ M or less, about 10.6 M or less, about 10⁻⁷ M or less, about 10⁻⁸ M or less, about 10⁻⁹ M or less, about 10⁻¹⁰ M or less, about 10.11 M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻¹¹ M to about 10⁻¹⁵ M, from about 10⁻¹² M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻¹¹ M to about 10⁻¹⁴ M, from about 10⁻¹² M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, from about 10⁻¹⁰ M to about 10⁻¹³ M, from about 10⁻¹¹ M to about 10⁻¹³ M, or from about 10⁻¹² M to about 10⁻¹³ M. In exemplary embodiments, K_{D}1 and K_{D}2 are independently equal to or less than: about 100 nM, about 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In one exemplary embodiment, K_{D}1 and K_{D}2 are independently less than or equal to about 1 nM. In another exemplary embodiment, K_{D}1 and K_{D}2 are independently less than or equal to about 100 pM. In another exemplary embodiment, K_{D}1 and K_{D}2 are independently less than or equal to about 10 pM. In yet another exemplary embodiment of the method according to the invention, the designed repeat domain binds to each of the first and second target with K_{D}1 and K_{D}2 being independently less than or equal to about 1 pM.

In one embodiment, said designed repeat domain binds to one of said first and second targets with a first binding affinity and to the other of said first and second targets with a second binding affinity, wherein the ratio of said first binding affinity and said second binding affinity is between 1:1 and 1:105. In further embodiments, said ratio may be equal to about 1:1, 1:10, 1:102, 1:103, 1:104 or 1:105.

In one embodiment, said first and second targets are located on different molecules. In one embodiment, said first and second targets are epitopes located on different molecules. In one embodiment, said first and second targets are located on a same molecule. In one embodiment, said first and second targets are epitopes on a same molecule. In one embodiment, said first and second targets are different epitopes on a same molecule. In some preferred embodiments, said first target is a molecule that is present in a tumor microenvironment (such as, e.g., VEGF) and said second target molecule is the paratope of a CD3-binding domain (e.g., a (designed) ankyrin repeat domain with binding specificity for CD3).

In one embodiment, said first repeat domain is an ankyrin repeat domain and said second repeat domain is an ankyrin repeat domain.

In one embodiment, said designed repeat domain is an ankyrin repeat domain.

### Repeat Domains of the Invention

In one aspect, the invention provides a designed repeat domain obtainable or obtained by the method according to the invention.

In one aspect, the invention provides a designed repeat domain having a first binding specificity for a first target and a second binding specificity for a second target, wherein binding of said designed repeat domain to said first and second targets is mutually exclusive.

The designed repeat domain of the present invention is thus a bispecific molecule, i.e. having dual binding specificity for two, preferably different, targets. The mutually exclusive binding property of the designed repeat domain of the invention is illustrated in FIG. 2 and explained in detail above in the context of the production method of the invention as well as in the appended Examples.

In some preferred embodiments, said first target and/or said second target is/are (a) molecule(s) having biological activity (as defined herein above). Accordingly, in one embodiment, said designed repeat domain, when bound to said first target or said second target, inhibits a biological activity of the bound target. In other words, the binding of the designed repeat domain to the target substantially or significantly interferes or competes with the binding of the target to its natural binding partner, and thereby the biological activity of the target is inhibited by binding to the designed repeat domain of the invention. In an alternative embodiment, said designed repeat domain, when bound to said first target or said second target, induces a biological activity by binding the target (as described herein above).

In one embodiment, binding of said designed repeat domain to one of said first and second targets sterically hinders binding of said designed repeat domain to the other of said first and second targets.

In some embodiments, the binding affinity of said designed repeat domain to said first target may be described in terms of dissociation constant (K_{D}) values. In one embodiment, said designed repeat domain binds to said first target with a dissociation constant (K_{D}) below 10.5M. In some embodiments, said designed repeat domain binds to said first target with a K_{D} of about 10⁻⁵ M or less, about 10⁻⁶ M or less, about 10⁻⁷ M or less, about 108 M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻¹¹ M to about 10⁻¹⁵ M, from about 10⁻¹² M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻¹¹ M to about 10⁻¹⁴ M, from about 10⁻¹² M to about 10⁻¹⁴ M, from about 10.5 M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, from about 10⁻¹⁰ M to about 10⁻¹³ M, from about 10⁻¹¹ M to about 10⁻¹³ M, or from about 10⁻¹² M to about 10⁻¹³ M. In further embodiments, said designed repeat domain binds to said first target with a K_{D} value of, or less than: about 1000 nM, about 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In one exemplary embodiment, said designed repeat domain binds to said first target with a K_{D} value of less than or equal to about 1 nM. In another exemplary embodiment, said designed repeat domain binds to said first target with a K_{D} value of less than or equal to about 100 pM. In another exemplary embodiment, said designed repeat domain binds to said first target with a K_{D} value of less than or equal to about 10 pM. In yet another exemplary embodiment, said designed repeat domain binds to said first target with a K_{D} value of less than or equal to about 1 pM.

In some embodiments, the binding affinity of said designed repeat domain to said second target may be described in terms of dissociation constant (K_{D}) values. In one embodiment, said designed repeat domain binds to said second target with a dissociation constant (K_{D}) below 10⁻⁵M. In certain embodiments, said designed repeat domain binds to said first target with a K_{D} of about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻¹¹ M to about 10⁻¹⁵ M, from about 10⁻¹² M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻¹¹ M to about 10⁻¹⁴ M, from about 10⁻¹² M to about 10⁻¹⁴ M, from about 10.5 M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, from about 10⁻¹⁰ M to about 10⁻¹³ M, from about 10⁻¹¹ M to about 10⁻¹³ M, or from about 10⁻¹² M to about 10⁻¹³ M. In further embodiments, said designed repeat domain binds to said first target with a K_{D} value of, or less than: about 1000 nM, about 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In one exemplary embodiment, said designed repeat domain binds to said first target with a K_{D} value of less than or equal to about 1 nM. In another exemplary embodiment, said designed repeat domain binds to said first target with a K_{D} value of less than or equal to about 100 pM. In another exemplary embodiment, said designed repeat domain binds to said first target with a K_{D} value of less than or equal to about 10 pM. In yet another exemplary embodiment, said designed repeat domain binds to said first target with a K_{D} value of less than or equal to about 1 pM.

In some embodiments, the binding affinity of said designed repeat domain to each of said first and second target may be described in terms of dissociation constant (K_{D}) values. In such embodiments, K_{D}1 represents the dissociation constant between said designed repeat domain and said first target and K_{D}2 represents the dissociation constant between said designed repeat domain and said second target. In exemplary embodiments, said designed repeat domain binds to each of said first and second target with K_{D}1 and K_{D}2 being independently about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10.5 M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻¹¹ M to about 10⁻¹⁵ M, from about 10⁻¹² M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻¹¹ M to about 10⁻¹⁴ M, from about 10⁻¹² M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, from about 10⁻¹⁰ M to about 10⁻¹³ M, from about 10⁻¹¹ M to about 10⁻¹³ M, or from about 10⁻¹² M to about 10⁻¹³ M. In exemplary embodiments, K_{D}1 and K_{D}2 are independently equal to or less than: about 1000 nM, about 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In one exemplary embodiment, K_{D}1 and K_{D}2 are independently less than or equal to about 1 nM. In another exemplary embodiment, K_{D}1 and K_{D}2 are independently less than or equal to about 100 pM. In another exemplary embodiment, K_{D}1 and K_{D}2 are independently less than or equal to about 10 pM. In yet another exemplary embodiment of the method according to the invention, the designed repeat domain binds to each of the first and second target with K_{D}1 and K_{D}2 being independently less than or equal to about 1 pM.

In one embodiment, said designed repeat domain binds to one of said first and second targets with a first binding affinity and to the other of said first and second targets with a second binding affinity, wherein the ratio of said first binding affinity and said second binding affinity is between 1:1 and 1:105. In further embodiments, said ratio may be equal to about 1:1, 1:10, 1:102, 1:103, 1:104 or 1:105.

In one embodiment, said first and second targets are located on different molecules. In one embodiment, said first and second targets are epitopes located on different molecules. In one embodiment, said first and second targets are located on the same molecule. In one embodiment, said first and second targets are epitopes on the same molecule. In a preferred embodiment, said first and second targets are different epitopes on the same molecule. In some preferred embodiments, said first target is a molecule that is present in a tumor microenvironment (such as, e.g., VEGF) and said second target molecule is the paratope of a CD3-binding domain (e.g., a (designed) ankyrin repeat domain).

In some embodiments, said designed repeat domain comprises at least 1 repeat module, at least 2 repeat modules, at least 3 repeat modules, at least 4 repeat modules, at least 5 repeat modules or at least 6 repeat modules. In some embodiments, said designed repeat domain comprises between 1 and 6 repeat modules, between 2 and 6 repeat modules, between 3 and 6 repeat modules, between 4 and 6 repeat modules or between 5 and 6 repeat modules. In some embodiments, said designed repeat domain comprises an N-terminal capping module and/or a C-terminal capping module. In some embodiments, said designed repeat domain comprises an N-terminal capping module and/or a C-terminal capping module and at least 1 internal repeat module, at least 2 internal repeat modules, at least 3 internal repeat modules or at least 4 internal repeat modules. In some embodiments, designed repeat domain comprises an N-terminal capping module and/or a C-terminal capping module and between 1 and 4 internal repeat modules, between 1 and 3 internal repeat modules or between 1 and 2 internal repeat modules.

In one particular embodiment, said designed repeat domain comprises at least two repeat modules.

In another particular embodiment, said designed repeat domain comprises at least three repeat modules.

In another particular embodiment, said designed repeat domain comprises at least four repeat modules. In another particular embodiment, said designed repeat domain comprises at least five repeat modules. In another particular embodiment, said designed repeat domain comprises at least six repeat modules. In another particular embodiment, said designed repeat domain comprises at least seven repeat modules. In one particular embodiment, said designed repeat domain comprises an N-terminal capping module and/or a C-terminal capping module, and at least one internal repeat module. In one particular embodiment, said designed repeat domain comprises an N-terminal capping module and/or a C-terminal capping module, and at least two internal repeat modules. In one particular embodiment, said designed repeat domain comprises an N-terminal capping module and/or a C-terminal capping module, and at least three internal repeat modules. In one particular embodiment, said designed repeat domain comprises an N-terminal capping module and/or a C-terminal capping module, and at least four internal repeat modules.

In one particular embodiment, said designed repeat domain comprises an N-terminal capping module and/or a C-terminal capping module, and one internal repeat module. In one particular embodiment, said designed repeat domain comprises an N-terminal capping module and/or a C-terminal capping module, and two internal repeat modules. In one particular embodiment, said designed repeat domain comprises an N-terminal capping module and/or a C-terminal capping module, and three internal repeat modules. In one particular embodiment, said designed repeat domain comprises an N-terminal capping module and/or a C-terminal capping module, and four internal repeat modules. In any of the above embodiments, the “and” conjunction is preferred, i.e., said designed repeat domain preferably comprises an N-terminal capping module and a C-terminal capping module in addition to the internal repeat module(s).

Accordingly, in some embodiments, said designed repeat domain comprises an N-terminal capping module, a C-terminal capping module and at least one internal repeat module. In one embodiment, said designed repeat domain comprises an N-terminal capping module, a C-terminal capping module and one internal repeat module. In one embodiment, said designed repeat domain comprises an N-terminal capping module, a C-terminal capping module and two internal repeat modules. In one embodiment, said designed repeat domain comprises an N-terminal capping module, a C-terminal capping module and three internal repeat modules. In one embodiment, said designed repeat domain comprises an N-terminal capping module, a C-terminal capping module, and four internal repeat modules. In one embodiment, said designed repeat domain comprises an N-terminal capping module, a C-terminal capping module, and five internal repeat modules. In one embodiment, said designed repeat domain comprises an N-terminal capping module, a C-terminal capping module, and six internal repeat modules. In one embodiment, said designed repeat domain comprises an N-terminal capping module, a C-terminal capping module, and seven internal repeat modules.

In one embodiment, all internal repeat modules comprised in said designed repeat domain contribute to the first and/or second binding specificity. In some embodiment, said first and/or second binding specificity involves target interaction residues comprised in said internal repeat modules. In one embodiment, all repeat modules comprised in said designed repeat domain contribute to the first and/or second binding specificity. In some embodiment, said first and/or second binding specificity involves target interaction residues comprised in said repeat modules. In one embodiment, said designed repeat domain comprises at least one internal repeat module which contributes to binding to said first target, and wherein said designed repeat domain comprises at least one internal repeat module which contributes to binding to said second target.

In one embodiment, said designed repeat domain comprises at least one, at least two, at least three, at least four, or at least five target interaction residues which bind to said first target, and/or wherein said designed repeat domain comprises at least one at least two, at least three, at least four, or at least five target interaction residues which bind to said second target. In one embodiment, said designed repeat domain comprises at least one, at least two, at least three, at least four, or at least five target interaction residues which bind to said first target, and wherein said designed repeat domain comprises at least one, at least two, at least three, at least four, or at least five target interaction residues which bind to said second target. Any combination in the above embodiment is possible, for instance, in one embodiment, said designed repeat domain comprises at least one target interaction residue which binds to said first target, and wherein said designed repeat domain comprises at least one target interaction residue which binds to said second target Methods to determine such contribution or the amino acid residues involved in the binding interaction between proteins or between a protein and a peptide, such as, e.g., alanine scanning mutagenesis, are well known to the person skilled in the art.

In one preferred embodiment, said designed repeat domain is an ankyrin repeat domain.

In one embodiment, said designed repeat domain comprises at least one internal repeat module comprising a sequence selected from SEQ ID NOs: 75 to 81.

In one embodiment, said designed repeat domain comprises (i) an N-terminal capping module comprising a sequence selected from the group consisting of (1) SEQ ID NOS: 35, 36, 47 to 63 and 83 to 85 and (2) sequences in which up to 9, or up to 8, or up to 7, or up to 6, or up to 5, or up to 4, or up to 3, or up to 2, or up to 1 amino acids in any of SEQ ID NOs: 35, 36, 47, 53, 59, 60, 83 and 84 are substituted by another amino acid; and/or (ii) a C-terminal capping module comprising a sequence selected from the group consisting of (1) SEQ ID NOS: 37, 38 and 64 to 74 and (2) sequences in which up to 9, or up to 8, or up to 7, or up to 6, or up to 5, or up to 4, or up to 3, or up to 2, or up to 1 amino acids in any of SEQ ID NOs: 37, 38 and 69 are substituted by another amino acid. Thus, in one embodiment, said designed repeat domain comprises (i) an N-terminal capping module comprising a sequence selected from the group consisting of (1) SEQ ID NOS: 35, 36, 47 to 63 and 83 to 85 and (2) sequences in which up to 9 amino acids in any of SEQ ID NOS: 35, 36, 47, 53, 59, 60, 83 and 84 are substituted by another amino acid; and/or (ii) a C-terminal capping module comprising a sequence selected from the group consisting of (1) SEQ ID NOS: 37, 38 and 64 to 74 and (2) sequences in which up to 9 amino acids in any of SEQ ID NOs: 37, 38 and 69 are substituted by another amino acid.

In one embodiment, said designed repeat domain comprises (i) an N-terminal capping module comprising a sequence selected from the group consisting of (1) SEQ ID NOS: 35, 36, 47 to 63, 83 to 85 and 97 to 100 and (2) sequences in which up to 9, or up to 8, or up to 7, or up to 6, or up to 5, or up to 4, or up to 3, or up to 2, or up to 1 amino acids in any of SEQ ID NOs: 35, 36, 47, 53, 59, 60, 83 and 84 are substituted by another amino acid; and/or (ii) a C-terminal capping module comprising a sequence selected from the group consisting of (1) SEQ ID NOS: 37, 38 and 64 to 74 and (2) sequences in which up to 9, or up to 8, or up to 7, or up to 6, or up to 5, or up to 4, or up to 3, or up to 2, or up to 1 amino acids in any of SEQ ID NOs: 37, 38 and 69 are substituted by another amino acid. Thus, in one embodiment, said designed repeat domain comprises (i) an N-terminal capping module comprising a sequence selected from the group consisting of (1) SEQ ID NOs: 35, 36, 47 to 63, 83 to 85 and 97 to 100, and (2) sequences in which up to 9 amino acids in any of SEQ ID NOs: 35, 36, 47, 53, 59, 60, 83 and 84 are substituted by another amino acid; and/or (ii) a C-terminal capping module comprising a sequence selected from the group consisting of (1) SEQ ID NOs: 37, 38 and 64 to 74 and (2) sequences in which up to 9 amino acids in any of SEQ ID NOs: 37, 38 and 69 are substituted by another amino acid.

In one embodiment, said designed repeat domain comprises (i) an N-terminal capping module comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 35, 36, 47, 53, 59, 60, 83 and 84; and/or (ii) a C-terminal capping module comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 37, 38 and 69.

In one embodiment, said designed repeat domain comprises at least one internal repeat module comprising a sequence selected from the group consisting of (1) SEQ ID NOs: 40 to 46 and (2) sequences in which up to 9, or up to 8, or up to 7, or up to 6, or up to 5, or up to 4, or up to 3, or up to 2, or up to 1 amino acids in any of SEQ ID NOs: 40 to 46 are substituted by another amino acid. Thus, in one embodiment, said designed repeat domain comprises at least one internal repeat module comprising a sequence selected from the group consisting of (1) SEQ ID NOS: 40 to 46 and (2) sequences in which up to 9 amino acids in any of SEQ ID NOS: 40 to 46 are substituted by another amino acid. In one embodiment, said designed repeat domain comprises at least one internal repeat module comprising a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 40 to 46.

In one embodiment, said designed repeat domain comprises at least one internal repeat module comprising a sequence selected from the group consisting of (1) SEQ ID NOs: 40 to 46 and (2) sequences in which up to 9, or up to 8, or up to 7, or up to 6, or up to 5, or up to 4, or up to 3, or up to 2, or up to 1 amino acids in any of SEQ ID NOS: 40 to 46 are substituted by another amino acid; and wherein said designed repeat domain further comprises:
(i) an N-terminal capping module comprising a sequence selected from the group consisting of (1) SEQ ID NOs: 35 to 36 and (2) sequences in which up to 9, or up to 8, or up to 7, or up to 6, or up to 5, or up to 4, or up to 3, or up to 2, or up to 1 amino acids in any of SEQ ID NOS: 35 to 36 are substituted by another amino acid; and/or
(ii) a C-terminal capping module comprising a sequence selected from the group consisting of (1) SEQ ID NO: 38 and (2) sequences in which up to 9, or up to 8, or up to 7, or up to 6, or up to 5, or up to 4, or up to 3, or up to 2, or up to 1 amino acids in SEQ ID NO: 38 are substituted by another amino acid.

Thus, in one embodiment, said designed repeat domain comprises at least one internal repeat module comprising a sequence selected from the group consisting of (1) SEQ ID NOs: 40 to 46 and (2) sequences in which up to 9 amino acids in any of SEQ ID NOs: 40 to 46 are substituted by another amino acid; and wherein said designed repeat domain further comprises:
(i) an N-terminal capping module comprising a sequence selected from the group consisting of (1) SEQ ID NOs: 35 to 36 and (2) sequences in which up to 9 amino acids in any of SEQ ID NOS: 35 to 36 are substituted by another amino acid; and/or
(ii) a C-terminal capping module comprising a sequence selected from the group consisting of (1) SEQ ID NO: 38 and (2) sequences in which up to 9 amino acids in SEQ ID NO: 38 are substituted by another amino acid.

In one embodiment, said designed repeat domain comprises at least one internal repeat module comprising a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 40 to 46; and wherein said designed repeat domain further comprises:
(i) an N-terminal capping module comprising a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 35 to 36; and/or
(ii) a C-terminal capping module comprising a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NO: 38.

In one embodiment, said designed repeat domain comprises:
(i) an N-terminal capping module comprising a sequence selected from the group consisting of (1) SEQ ID NOs: 35 to 36 and (2) sequences in which up to 9, or up to 8, or up to 7, or up to 6, or up to 5, or up to 4, or up to 3, or up to 2, or up to 1 amino acids in any of SEQ ID NOs: 35 to 36 are substituted by another amino acid; and/or
(ii) a C-terminal capping module comprising a sequence selected from the group consisting of (1) SEQ ID NO: 38 and (2) sequences in which up to 9, or up to 8, or up to 7, or up to 6, or up to 5, or up to 4, or up to 3, or up to 2, or up to 1 amino acids in SEQ ID NO: 38 are substituted by another amino acid.

Thus, in one embodiment, said designed repeat domain comprises:
(i) an N-terminal capping module comprising a sequence selected from the group consisting of (1) SEQ ID NOs: 35 to 36 and (2) sequences in which up to 9 amino acids in any of SEQ ID NOs: 35 to 36 are substituted by another amino acid; and/or
(ii) a C-terminal capping module comprising a sequence selected from the group consisting of (1) SEQ ID NO: 38 and (2) sequences in which up to 9 amino acids in SEQ ID NO: 38 are substituted by another amino acid.

In one embodiment, said designed repeat domain comprises:
(i) an N-terminal capping module comprising a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 35 to 36; and/or
(ii) a C-terminal capping module comprising a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NO: 38.

In any of the above embodiments relating to amino acid substitutions in repeat modules, said substitutions can occur at positions of target interaction residues and/or positions of framework residues. Accordingly, in some embodiments said substitutions can occur at positions of target interaction residues. In other embodiments, said substitutions can occur at positions of framework residues. In other embodiments, said substitutions can occur both at positions of target interaction residues and positions of framework residues.

More particularly, in internal repeat modules, positions of target interaction residues are positions corresponding to positions 3, 4, 6, 11, 14 and 15 of SEQ ID NO: 39 and positions of framework residues are positions corresponding to positions other than said positions 3, 4, 6, 11, 14 and 15 of SEQ ID NO: 39. Accordingly, in any of the the above embodiments relating to amino acid substitutions in internal repeat modules, said substitutions can occur at any such position of target interaction residue and/or position of framework residue.

In N-terminal capping modules, positions of target interaction residues are positions corresponding to positions 4, 5, 8, 11 and 12 of SEQ ID NO: 36 and positions of framework residues are positions corresponding to positions other than said positions 4, 5, 8, 11 and 12 of SEQ ID NO: 36. Accordingly, in any of the the above embodiments relating to amino acid substitutions in N-terminal capping modules, said substitutions can occur at any such position of target interaction residue and/or position of framework residue.

In C-terminal capping modules, positions of target interaction residues are positions corresponding to positions 3, 4, 6, 14 and 15 of SEQ ID NO: 38 and positions of framework residues are positions corresponding to positions other than said positions 3, 4, 6, 14 and 15 of SEQ ID NO: 38. Accordingly, in any of the the above embodiments relating to amino acid substitutions in C-terminal capping modules, said substitutions can occur at any such position of target interaction residue and/or position of framework residue.

In one embodiment, said designed repeat domain comprises at least one internal repeat module, wherein residues at positions corresponding to positions 3, 4, 6, 11, 14 and/or 15 of SEQ ID NO: 39 are target interaction residues, and wherein a residue at a position corresponding to position 10 of SEQ ID NO: 39 is a framework residue.

In one embodiment, said designed repeat domain comprises at least one internal repeat module, wherein residues at positions corresponding to positions 3, 4, 6, 11, 14 and/or 15 of SEQ ID NO: 39 are target interaction residues.

In one embodiment, said designed repeat domain comprises at least one N-terminal capping module, wherein residues at positions corresponding to positions 4, 5, 8, 11 and/or 12 of SEQ ID NO: 36 are target interaction residues, and wherein a residue at a position corresponding to position 7 of SEQ ID NO: 36 is a framework residue.

In one embodiment, said designed repeat domain comprises at least one N-terminal capping module, wherein residues at positions corresponding to positions 4, 5, 8, 11 and/or 12 of SEQ ID NO: 36 are target interaction residues.

In one embodiment, said designed repeat domain comprises at least one C-terminal capping module, wherein residues at positions corresponding to positions 3, 4, 6, 14 and/or 15 of SEQ ID NO: 38 are target interaction residues, and wherein residues at positions corresponding to positions 10 and/or 11 of SEQ ID NO: 38 are framework residues.

In one embodiment, said designed repeat domain comprises at least one C-terminal capping module, wherein residues at positions corresponding to positions 3, 4, 6, 14 and/or 15 of SEQ ID NO: 38 are target interaction residues.

In some embodiments, said designed repeat domains according to the invention comprise a paratope. Such paratope is formed by the repeat modules comprised in said repeat domain, and more precisely by the target interaction residues comprised in each of these repeat modules. Therefore, in some embodiments, such paratope is essentially separated in two regions, where each of said region comprises repeat modules contributing to the first or second binding specificity. In such embodiments, the paratope regions do not overlap. In other embodiments, some repeat modules may contribute to both binding specificities due to one or more target interaction residues which interact with both targets, and therefore both regions of the paratope may overlap to a certain extent.

In one embodiment, said designed repeat domain comprises two internal repeat modules, wherein the N-terminal one of said two internal repeat modules comprises target interaction residues which bind to said first target, and wherein the C-terminal one of said two internal repeat modules comprises target interaction residues which bind to said second target.

In one embodiment, said designed repeat domain comprises three internal repeat modules, wherein the most N-terminal one of said three internal repeat modules comprises target interaction residues which bind to said first target, and wherein the most C-terminal one of said three internal repeat modules comprises target interaction residues which bind to said second target, and wherein the middle one of said three internal repeat modules comprises target interaction residues which bind to said first and/or second target(s). In a particular embodiment, said designed repeat domain comprises an amino acid sequence selected from the group consisting of (1) SEQ ID NOs: 6 to 10 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 6 to 10. Thus, in a more particular embodiment, said designed repeat domain comprises an amino acid sequence selected from the group consisting of (1) SEQ ID NOs: 6 to 10 and (2) sequences with at least 80% amino acid sequence identity with any one of SEQ ID NOs: 6 to 10.

In one embodiment, said designed repeat domain comprises four internal repeat modules, wherein the most N-terminal one of said four internal repeat modules comprises target interaction residues which bind to said first target, wherein the most C-terminal one of said four internal repeat modules comprises target interaction residues which bind to said second target, and wherein each of the second-most N-terminal one and the second-most C-terminal one of said four internal repeat modules comprises target interaction residues which bind to said first target and/or said second target. In a particular embodiment, said designed repeat domain comprises an amino acid sequence selected from the group consisting of (1) SEQ ID NOS: 11 to 13 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 11 to 13. Thus, in a more particular embodiment, said designed repeat domain comprises an amino acid sequence selected from the group consisting of (1) SEQ ID NOS: 11 to 13 and (2) sequences with at least 80% amino acid sequence identity with any one of SEQ ID NOS: 11 to 13.

In one embodiment, said designed repeat domain comprises six internal repeat modules, wherein the three most N-terminal ones of said six internal repeat modules comprise target interaction residues which bind to said first target, wherein the two most C-terminal ones of said six internal repeat modules comprise target interaction residues which bind to said second target, and wherein the fourth most N-terminal one (or the third most C-terminal one) of said six internal repeat modules comprise target interaction residues which bind to said first target and/or said second target. In a particular embodiment, said designed repeat domain comprises an amino acid sequence selected from the group consisting of (1) SEQ ID NO: 92 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 92. Thus, in a more particular embodiment, said designed repeat domain comprises an amino acid sequence selected from the group consisting of (1) SEQ ID NO: 92 and (2) sequences with at least 80% amino acid sequence identity with SEQ ID NO: 92.

In one embodiment, said designed repeat domain comprises seven internal repeat modules, wherein the three most N-terminal ones of said seven internal repeat modules comprise target interaction residues which bind to said first target, wherein the two most C-terminal ones of said seven internal repeat modules comprise target interaction residues which bind to said second target, and wherein each of the fourth and fifth most N-terminal ones (or each of the third and fourth most C-terminal ones) of said seven internal repeat modules comprise target interaction residues which bind to said first target and/or said second target. In a particular embodiment, said designed repeat domain comprises an amino acid sequence selected from the group consisting of (1) SEQ ID NOs: 90, 91, 93 and 94 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with any one of SEQ ID NOs: 90, 91, 93 and 94. Thus, in a more particular embodiment, said designed repeat domain comprises an amino acid sequence selected from the group consisting of (1) SEQ ID NOs: 90, 91, 93 and 94 and (2) sequences with at least 80% amino acid sequence identity with any one of SEQ ID NOs: 90, 91, 93 and 94.

Furthermore, the sequence of any repeat domain of the present invention may optionally comprise at its N-terminus, a G, an S, or a GS. Furthermore, the sequence of any repeat domain of the present invention may optionally have A at the second last position substituted with L and/or A at the last position substituted with N.

### Recombinant Proteins

The designed repeat domains of the invention can be genetically fused to further components, such as, e.g., a therapeutic agent, and such fusions are also referred to as “recombinant protein” or “constructs” interchangeably. In such constructs, the designed repeat domain may act as on- or off-switch for a fused therapeutic agent.

FIG. 3 shows generic examples of constructs comprising a designed repeat domain of the invention. These exemplary constructs are also referred to as “Switchdrug DARPins”. In the on-switch setup (FIG. 3A), a designed repeat domain of the invention binds target A (e.g. a sensor target) or target B (e.g. a drug molecule) in a mutually exclusive manner, and is chemically linked to said target B (e.g. a drug molecule). The state of the target B (e.g. active or inactive drug molecule) as part of the Switchdrug DARPin is conditionally regulated by the interaction of the designed repeat domain of the invention with target A, wherein binding of target A by the designed repeat domain of the invention results in release of bound target B (hence, active drug molecule). In an exemplary off-switch setup shown in FIG. 3B, the designed repeat domain of the invention is designed to bind target A (e.g. a sensor target) or target B (e.g. a cell receptor). A downstream activity resulting from the interaction of the designed repeat domain of the invention with target B can be conditionally switched-off upon binding of target A to the designed repeat domain of the invention. The designed repeat domain of the invention can be further linked to other binding domains or molecules.

Accordingly, in one aspect, the invention provides a recombinant protein comprising the designed repeat domain of the invention. In one embodiment, said protein further comprises a therapeutic agent. In one embodiment, said therapeutic agent is a binding molecule. In a further embodiment, said binding molecule comprises a designed ankyrin repeat domain.

Such recombinant proteins of the invention can be generated in different formats. In one embodiment, said therapeutic agent is fused on the N-terminal side of the designed repeat domain of the invention. In one embodiment, said therapeutic agent is fused on the C-terminal side of the designed repeat domain of the invention. In some embodiments, said therapeutic agent is directly linked to the designed repeat domain of the invention. In some embodiments, a peptide linker is used to link the designed repeat domain of the invention to said therapeutic agent. In some embodiments, said therapeutic agent is separated from the designed repeat domain of the invention by further molecules such as or one or more ankyrin repeat domains.

In one embodiment, the recombinant protein of the invention comprises additional repeat domains, for example the recombinant protein of the invention comprises the designed repeat domain of the invention and two or more further ankyrin repeat domains, said two or more ankyrin repeat domains, for example said two or three ankyrin repeat domains, may be linked with a peptide linker. In one embodiment, said peptide linker is a proline-threonine rich peptide linker. In one embodiment, said peptide linker is the proline-threonine rich peptide linker of SEQ ID NO: 82. Other linkers known in the art may also be used to link repeat domains (see, e.g., WO 2021/116469).

In some embodiments, each of the first and second binding regions (or paratopes) comprised in the designed repeat domain of the invention may have a defined function. In one embodiment, said designed repeat domain is described as comprising a sensor part and a blocker part, wherein said sensor part corresponds to one of the first or second binding regions and said blocker part corresponds to the other binding region of the repeat domain. Consequently, in such constructs, the designed repeat domain of the invention has a first binding specificity for a sensor target and a second binding specificity for a blocker target. In preferred embodiments, said blocker target is a therapeutic agent and said sensor target can be chosen as needed depending on the therapeutic agent, application or context. Based on the selected therapeutic agent and sensor target, designed repeat domains can be created as described in the present disclosure, and subsequently genetically fused to further components, in particular to the therapeutic agent, using methods know to a person skilled in the art.

Immune cell engagers such as T-cell engagers (TCEs) or Natural Killer (NK)-cell engagers can be advantageously used as fusion partners in the recombinant proteins of the invention. A TCE typically comprises a CD3-binding domain and a tumor-associated antigen (TAA)-binding domain. Accordingly, in one preferred embodiment, the recombinant protein of the invention comprises the designed repeat domain of the invention having a first binding specificity for the α-CD3 binding domain of the TCE (blocker target) and a second binding specificity for a sensor target. Said designed repeat domain binds the TCE and inhibits its activity as long as said sensor target is not bound to said designed repeat domain. High concentrations of the sensor target induce the release of the CD3-binding domain (blocker target), enabling the recruitment of cytotoxic T-cells and the formation of an immunological synapse between a TAA-expressing tumor cell and a T-cell. Such an embodiment is represented in FIG. 4A.

The examples disclosed herein exemplify various recombinant protein (or constructs) comprising a TCE which comprises a CD3-binding domain, preferably a CD3-binding DARPin, and optionally a CD123-binding or non-binding (placeholder) domain, and the designed repeat domain of the invention with a first binding specificity for the paratope of said CD3-binding domain (blocker target) and a second binding specificity for VEGF (sensor target). In some embodiments, the affinity of the designed repeat domain of the invention for the sensor target is higher than the affinity of said repeat domain for the blocker target.

Accordingly, in one embodiment, the therapeutic agent described herein is a binding molecule, wherein said binding molecule is an immune cell engager. In a particular embodiment, said binding molecule is a TCE.

In one embodiment, the recombinant protein of the invention comprises an α-CD3-binding domain on the N-terminal side of the designed repeat domain of the invention. In one embodiment, the recombinant protein of the invention comprises an α-CD3-binding domain on the C-terminal side of the designed repeat domain of the invention. In a preferred embodiment, the recombinant protein of the invention comprises from N- to C-terminus: the designed repeat domain of the invention, another binding or non-binding domain and an α-CD3-binding domain. In an alternative preferred embodiment, the recombinant protein of the invention comprises from N- to C-terminus: a TAA-binding domain, an α-CD3-binding domain and the designed repeat domain of the invention. In a more preferred embodiment, the recombinant protein of the invention comprises from N- to C-terminus: the designed repeat domain of the invention, a TAA-binding domain and an α-CD3-binding domain. In particular embodiments, said TAA-binding domain has the amino acid sequence of CD123-binding domain as set forth in SEQ ID NO: 4. In particular embodiments, said non-binding domain has the amino acid sequence as set forth in SEQ ID NO: 5.

In one embodiment, the recombinant protein of the invention comprises an amino acid sequence selected from the group consisting of (1) SEQ ID NOS: 15 to 30 and (2) sequences that have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% amino acid sequence identity with any of SEQ ID NOS: 15 to 30. In one embodiment, said recombinant protein comprises an amino acid sequence selected from the group consisting of (1) SEQ ID NOS: 15 to 30 and (2) sequences that have at least 80% amino acid sequence identity with any of SEQ ID NOs: 15 to 30.

In other embodiments, said designed repeat domain is described as comprising a sensor part and an effector part, wherein said sensor part corresponds to one of the first or second binding regions and said effector part corresponds to the other binding region of the repeat domain. Consequently, in such constructs, the repeat domain of the invention has a first binding specificity for a sensor target and a second binding specificity for an effector target. In some embodiments, said effector target is a molecule inducing an effect to a living cell. In some embodiments, said effector target is a cell surface molecule. In some embodiments, said sensor target can be chosen as needed depending on the effector target, application or context. Based on the selected effector target and sensor target, designed repeat domains can be created as described in the present disclosure, and optionally fused to further components using methods know to a skilled person in the art.

Accordingly, in one embodiment, the recombinant protein of the invention comprises the designed repeat domain of the invention having a first binding specificity for an effector target and a second binding specificity for a sensor target. As long as said designed repeat domain is bound to the effector target, the effector target remains active. Upon high concentrations of the sensor target, binding of said sensor target to said designed repeat domain prevents activation of the effector target by preventing interaction between said repeat domain and said effector target. In such embodiments, the designed repeat domains can be used as off-switch, as also represented in FIGS. 3B and 4B.

### Nucleic Acids, Vectors and Host Cells

In another aspect, the invention relates to an isolated nucleic acid encoding the amino acid sequence of the designed repeat domain of the invention or of the recombinant protein of the invention. In one embodiment, the invention relates to an isolated nucleic acid encoding the amino acid sequence of the recombinant protein of the present invention. In one embodiment, the invention relates to an isolated nucleic acid encoding the amino acid sequence of the designed repeat domain of the present invention.

Furthermore, the invention relates to vectors comprising any nucleic acid of the invention. Accordingly, in another aspect, the invention provides a recombinant expression vector comprising a nucleic acid according to the invention, wherein the vector optionally comprises an expression control sequence, allowing expression in prokaryotic or eukaryotic host cells of the encoded polypeptide, operably linked to said nucleic acid. The nucleic acid sequence can be inserted in the recombinant vector by methods well known to a person skilled in the art such as, for example, those that are described in MOLECULAR CLONING: A LABORATORY MANUAL, Sambrook et al, 4th Ed., Cold Spring Harbor Laboratory Press, Cold Spring Harbor, N Y., 2001.

Nucleic acids are well known to the skilled person in the art. Nucleic acids were used to produce designed ankyrin repeat domains or recombinant binding proteins of the invention in E. coli, e.g. as described in U.S. Pat. No. 7,417,130.

In another aspect, the invention provides a host cell comprising a recombinant expression vector according to the invention. The host cell can be, for example, bacterial cells such as Escherichia coli or Streptomyces, fungal cells such as Aspergillus and yeasts such as Saccharomyces, insect cells, mammalian cells such as Chinese Hamster Ovary (CHO) cells, CI 27 mouse cell line, BHK cell line of Syrian hamster cells, Human Embryonic Kidney 293 (HEK 293) cells. In some embodiment, the host cell is a CHO cell or a HEK 293 cell. The host cells can be used, for example, to express a recombinant protein of the invention.

### Compositions

The invention further relates to pharmaceutical compositions comprising one or more of a designed repeat domain, a recombinant protein, a nucleic acid and/or a recombinant expression vector described herein and a pharmaceutically acceptable carrier or diluent. The invention also relates to uses and methods of treatment using said pharmaceutical compositions disclosed herein. The methods and uses encompassed by the present invention are described in more detail below.

As presented above, designed repeat domains of the invention can be used to provide a conditional activation and/or deactivation mechanism for a therapeutic agent (e.g. on-switch or off-switch mechanisms). Thus, in some embodiments, the designed repeat domains do not substantially alter the biological effect of the therapeutic agent in-vivo, other than to inhibit the therapeutic agent (blocker target of the designed repeat domain) as long as the sensor target of the designed repeat domain is not present and substantially bound to said designed repeat domain.

The pharmaceutical compositions described herein may be prepared using methods known in the art.

The pharmaceutical compositions optionally comprise a pharmaceutically acceptable carrier or excipient or diluent. Standard pharmaceutical carriers include a phosphate buffered saline solution, water, emulsions such as an oil/water or water/oil emulsion, and various types of wetting agents.

The pharmaceutical compositions may comprise any other pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, colouring agents, desiccants, detergents, diluents, disinfectants, disintegrants, dispersing agents, dissolution enhancing agents, dyes, emollients, emulsifying agents, emulsion stabilizers, fillers, film forming agents, flavour enhancers, flavouring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety. In one embodiment, the invention provides a pharmaceutical composition comprising one or more of: (i) a designed repeat domain according to the invention, (ii) a recombinant protein according to the invention, (iii) a nucleic acid according to the invention, and/or (iv) a recombinant expression vector according to the invention, and at least one pharmaceutically acceptable carrier or diluent.

### Therapeutic Uses and Methods According to the Invention

In the context of the invention, the terms “medical condition”, “disease” and “disorder” are used interchangeably and include but are not limited to cancer. In one preferred embodiment, said medical condition is a cancer.

In another aspect, the invention provides a method of treating a medical condition, the method comprising the step of administering to a patient in need thereof a therapeutically effective amount of the designed repeat domain of the invention, the recombinant protein of the invention, the nucleic acid of the invention or the pharmaceutical composition of the invention.

Further provided is the designed repeat domain, the recombinant protein, the nucleic acid, or the pharmaceutical composition of the invention for use in a method of treating a medical condition.

In one embodiment, the invention relates to the use of the designed repeat domain, the recombinant protein, the nucleic acid, or the pharmaceutical composition according to the present invention for the treatment of a disease. For that purpose, the designed repeat domain, the recombinant protein, the nucleic acid, or the pharmaceutical composition according to the present invention is administered to a patient in need thereof, in a therapeutically effective amount.

In one embodiment, the invention relates to a method of treatment of a medical condition, the method comprising the step of administering, to a patient in need of such a treatment, a therapeutically effective amount of the designed repeat domain, recombinant protein, nucleic acid or pharmaceutical composition of the invention.

In one embodiment, the invention relates to the use of the designed repeat domain, recombinant protein, nucleic acid or pharmaceutical composition of the present invention for the treatment of a medical condition. In one embodiment, the invention relates to the designed repeat domain, recombinant protein, nucleic acid or pharmaceutical composition of the invention for use in the treatment of a medical condition.

In one embodiment, the invention relates to the use of the designed repeat domain, recombinant protein, nucleic acid or pharmaceutical composition of the invention, as a medicament for the treatment of a medical condition.

In one embodiment, the invention relates to a process of treatment of a medical condition using the designed repeat domain, recombinant protein, nucleic acid or pharmaceutical composition of the invention. In one embodiment, the invention relates to a process for the manufacturing of a medicament for the treatment of a medical condition, wherein the designed repeat domain, recombinant protein, nucleic acid or pharmaceutical composition of the invention is an active ingredient of the medicament.

In one embodiment, the invention relates to the use of the designed repeat domain, recombinant protein, nucleic acid or pharmaceutical composition of the invention, for manufacturing of a medicament.

In one embodiment, the invention relates to the use of the designed repeat domain, recombinant protein, nucleic acid or pharmaceutical composition of the invention, for manufacturing of a medicament for the treatment of a medical condition.

In a further embodiment, the invention relates to the use of the designed repeat domain, recombinant protein, nucleic acid or pharmaceutical composition of the invention for the manufacture of a medicament that is used for the treatment of a medical condition, preferably a neoplastic disease, more preferably cancer.

In some preferred embodiments, said medical condition is a cancer.

In some embodiments, said patient is a mammal. In preferred embodiments, the patient is a human.

In some embodiments, a single administration of said designed repeat domain, recombinant protein, nucleic acid or pharmaceutical composition of the invention may be sufficient. In other embodiments, repeated administration may be necessary. Various factors will impact on the number and frequency of administrations, such as the age and general health of the subject, as well as the nature and typical dosage regime of the therapeutic agent.

The designed repeat domain, recombinant protein, nucleic acid or pharmaceutical compositions described herein may be used in combination with another therapeutic agent. Each therapeutic agent may be administered simultaneously (e.g., in the same medicament or at the same time), concurrently (i.e., in separate medicaments administered one right after the other in any order) or sequentially in any order. Sequential administration may be useful when the therapeutic agents in the combination therapy are in different dosage forms (e.g., one agent is a tablet or capsule and another agent is a sterile liquid) and/or are administered on different dosing schedules, e.g., an analgesic that is administered at least daily and a biotherapeutic that is administered less frequently, such as once weekly or once every two weeks. The invention is not restricted to the particular embodiments described in the Examples.

### EXAMPLES

Starting materials and reagents disclosed below are known to those skilled in the art, are commercially available and/or can be prepared using well-known techniques.

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA). NLC chips for SPR measurements were from BioRad (BioRad, USA). HTRF reagents were from Cisbio (Cisbio, France). Pan-T cell isolation kit was from Miltenyi Biotec (Germany). Detection antibody anti-hCD8 was from BD Pharmingen™ (BD, USA) and anti-hCD69 was from BioLegend (BioLegend, USA).

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module or a randomized N-terminal capping module, one or more randomized repeat modules, and a fixed C-terminal capping module or a randomized C-terminal capping module (see, e.g., the N-terminal capping modules and C-terminal capping modules provided in WO 2021/116462). Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complementarity determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23 (30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g. 1 to 20 amino acids) inserted in one or more beta-turns of an ankyrin repeat domain.

An N-terminal capping module of an ankyrin repeat protein library preferably possesses the RILLAA, RILLKA or RELLKA motif and any such C-terminal capping module of an ankyrin repeat protein library preferably possesses the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V20, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

### Example 1: Design of 2-in-1 DARPins

### Summary

DARPins (designed ankyrin repeat proteins) are repeat proteins comprising one or more internal repeat modules that are identical except for their randomized positions, and flanked by an N-terminal and a C-terminal capping repeat modules (Binz et al. 2003). As used herein, the term “repeat module” encompasses internal repeat modules and terminal repeat modules (C-cap and N-cap modules). 27 of their 33 amino acid positions are highly conserved, whereas the other 6 are less conserved and to the most part responsible for the interaction of the ankyrin repeat with its target (Binz et al. 2003). The paratope of the DARPin is formed by the continuous surface formed by these variable positions of the internal repeats and sometimes also the capping repeat modules. Hence, the DARPin structure offers an intrinsic modularity to shorten, elongate or fuse the paratope of one DARPin into the paratope of another DARPin.

As illustrated in FIG. 1, a first DARPin (1) binding target A, and a second DARPin (2) binding target B can be split after a desired number of repeat modules and genetically combined to a complementary fragment of the other DARPin. Their respective paratopes or binding surfaces are shown ((4) and (5)). In this way, a monodomain “2-in-1 DARPin” (or “SwitchDARPin” (6)) can be generated. Such 2-in-1 DARPin can bind the two targets A and B in a mutually exclusive manner, i.e. either target A or target B. Additionally, one or several internal repeat modules in the 2-in-1 DARPin can be mixed (or merged) repeat modules (7). A repeat module is called a mixed repeat module if its paratope comprises at least one target interaction residue from the corresponding module in the first parent repeat protein and at least one target interaction residue from the corresponding module in the second parent repeat protein. The mutually exclusive binding property of the 2-in-1 DARPin is illustrated in FIG. 2. Due to the close proximity or overlap of the two paratopes of the 2-in-1 DARPin, the molecule is able to bind target A (FIG. 2A) or target B (FIG. 2B) individually, but not both targets at the same time (FIG. 2C), as the simultaneous binding of both targets A and B is sterically hindered. This mutually exclusive binding feature in consequence enables the design of on- or off-switches for conditional activation or de-activation of drug molecules. This mutually exclusive binding feature also distinguishes a 2-in-1 DARPin from other ankyrin repeat protein formats (Mohan, K. et al, 2019, Science, 364 (6442); WO 2020/190852 A2).

FIG. 3 shows generic examples of constructs comprising a 2-in-1 DARPin of the invention. These exemplary constructs are also referred to as “Switchdrug DARPins”. In the on-switch setup (FIG. 3A), a 2-in-1 DARPin binds target A (e.g. a sensor target) or target B (e.g. a drug molecule) in a mutually exclusive manner, and is chemically linked to said target B (e.g. a drug molecule). The state of the target B (e.g. active or inactive drug molecule) as part of the Switchdrug DARPin is conditionally regulated by the interaction of the 2-in-1 DARPin with target A, wherein binding of target A by the 2-in-1 DARPin results in release of bound target B (hence, active drug molecule). In an exemplary off-switch setup shown in FIG. 3B, a 2-in-1 DARPin is designed to bind target A (e.g. a sensor target) or target B (e.g. a cell receptor). A downstream activity resulting from the interaction of said 2-in-1 DARPin with target B can be conditionally switched-off upon binding of target A to the 2-in-1 DARPin. Such a 2-in-1 DARPin can be further linked to other binding domains or molecules.

### T-Cell Engagers are Ideal Drug Molecules for 2-In-1 DARPin Proof-of-Concept

T-cell engagers (TCE, also called T-cell bispecifics) recruit T-cells to target cells and are known to be very potent drugs that have shown impressive clinical results. However, they often suffer from severe side effects like e.g. on-target, off-tumor toxicity. A 2-in-1 DARPin can be advantageously used to add another layer of safety to the activation of TCE, in order to reduce this on-target, off-tumor effect. In the on-switch concept (FIG. 4A), the T-cell engager comprises a CD3-binding DARPin (target B) and a tumor-associated antigen (TAA)-binding DARPin. A 2-in-1 DARPin is used which can bind and block the paratope of the CD3-binding DARPin, and which can alternatively bind to a molecule that is for instance predominantly present in the tumor-microenvironment, represented as target A. This 2-in-1 DARPin is chemically linked to the TCE to form a Switchdrug DARPin. Upon binding of target A, the 2-in-1 DARPin releases the CD3-binding domain of the TCE (target B), thus enabling it to bind to CD3, and thus leading to the formation of a trimolecular complex between T-cell, drug and target cell.

In the exemplary off-switch concept (FIG. 4B), a 2-in-1 DARPin is used which binds target A (a sensor target) and alternatively binds target B (CD3). The TCE in this case comprises a tumor-associated antigen (TAA)-binding DARPin chemically linked to said 2-in-1 DARPin. Upon high concentrations of target A, the TCE can be silenced by binding to target A, thereby preventing binding of the TCE to CD3 on T cells.

Design of 2-In-1 DARPins with Mutually Exclusive Binding Specificity for VEGF and the α-CD3 Binder of a TCE

Based on the on-switch concept illustrated in FIG. 4A, 2-in-1 DARPins resulting from the combination of a first VEGF-binding DARPin (SEQ ID NO: 1) and a second DARPin targeting the paratope of a CD3-binding DARPin, also referred to as “α-CD3 DARPin-blocker” (SEQ ID NO: 2) were designed. These two parent DARPin sequences are based on previous work: the VEGF-binder is disclosed in WO 2016/156596 and the α-CD3 DARPin binder was obtained as disclosed in U.S. provisional applications Nos. 63/126,356 and 63/182,394.

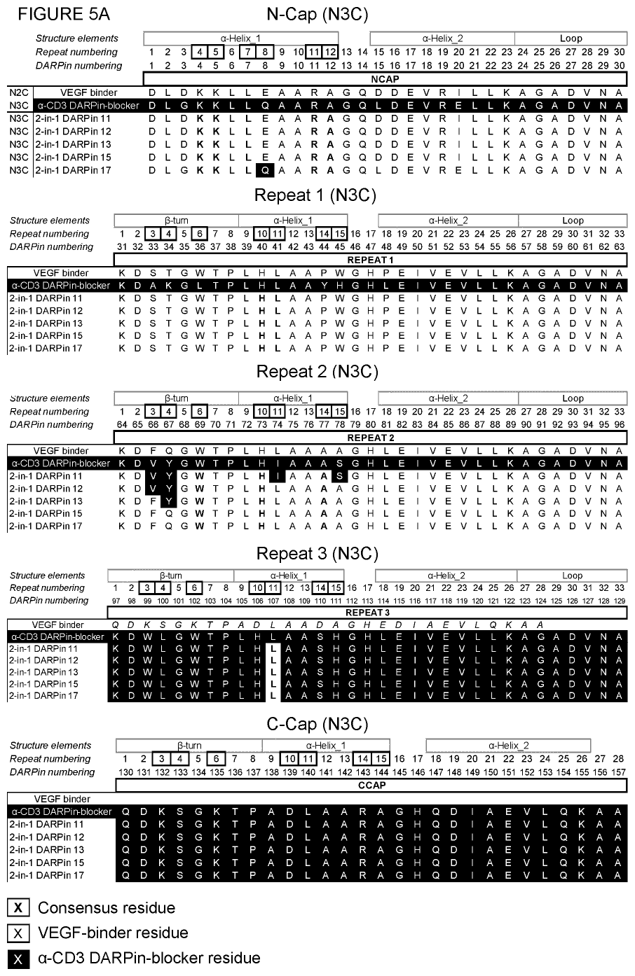

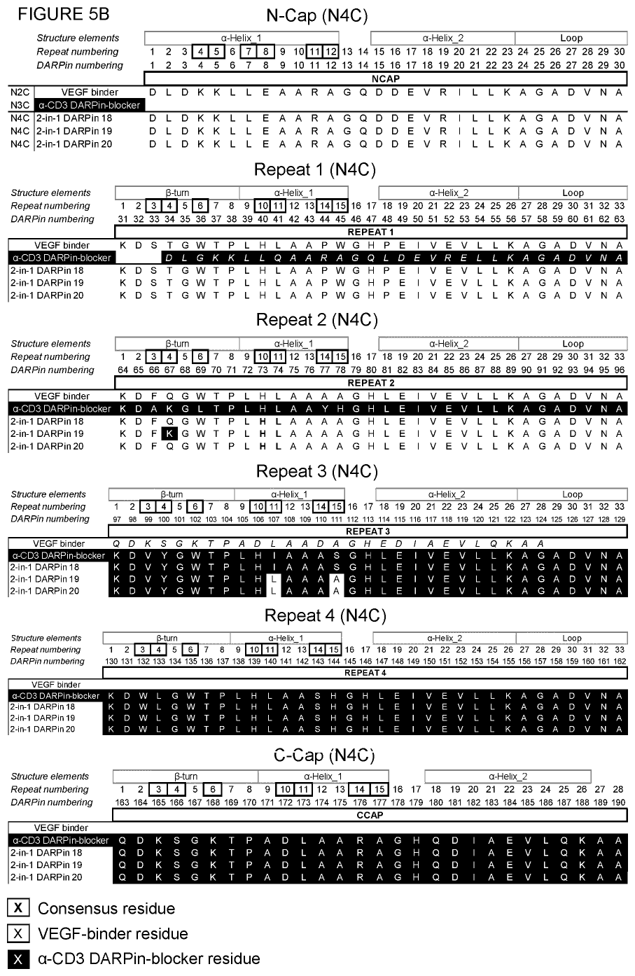

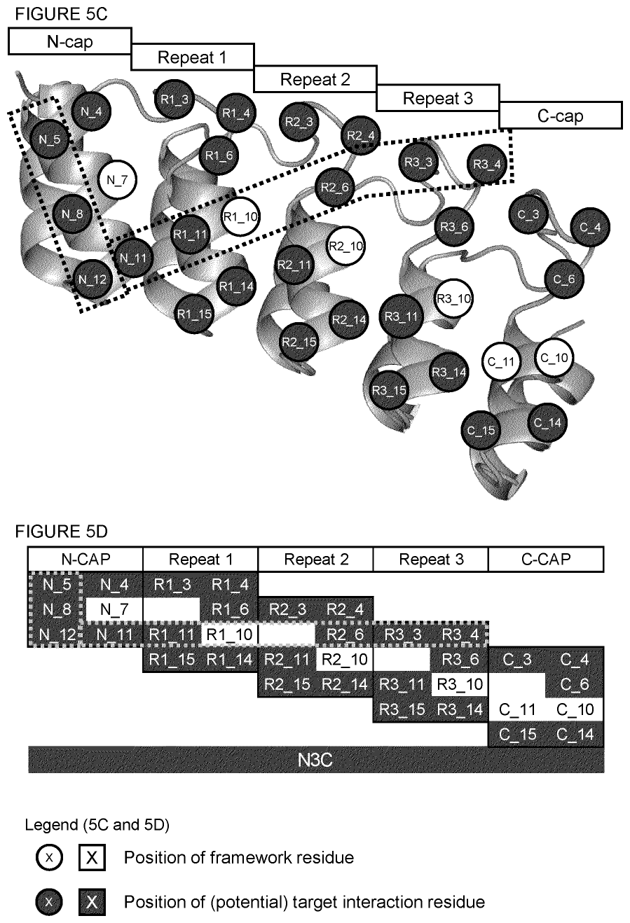

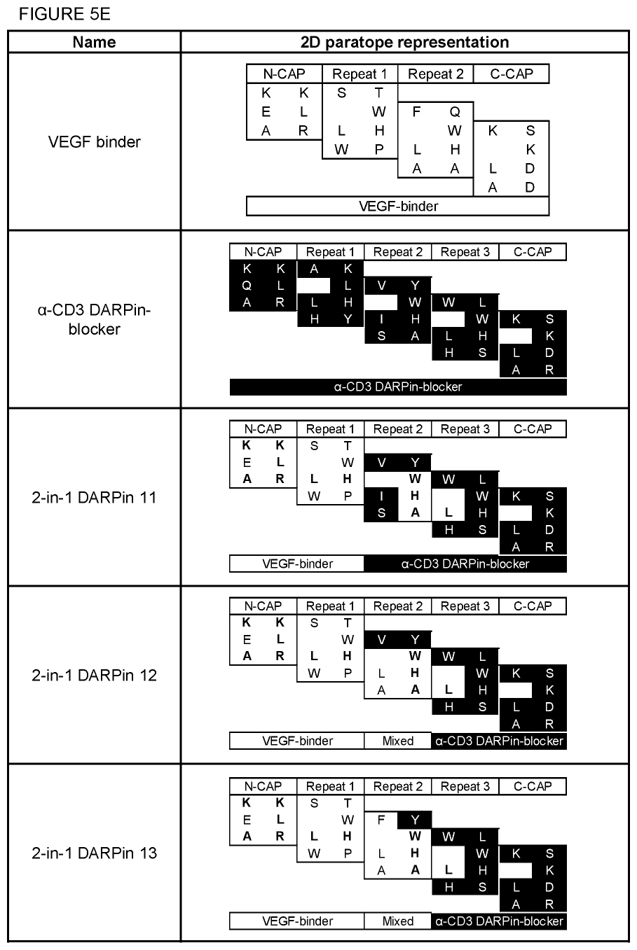

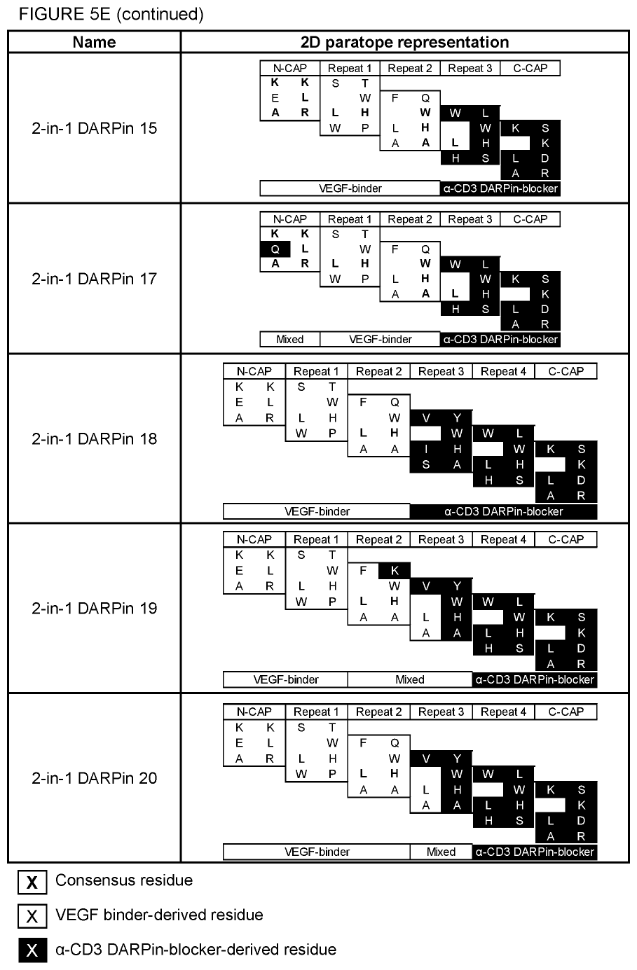

The 2-in-1 DARPins were designed by combining the N-terminal capping repeat and internal repeat module(s) originating from the VEGF-binder, and the C-terminal internal repeat and capping repeat modules originating from the α-CD3 DARPin-blocker (FIG. 5A-B). In total 29 constructs were designed by combining different numbers of repeats modules of VEGF-binder and α-CD3 DARPin-blocker. Said repeat modules where either combined as present in the parent binders (intact) or comprised further amino acid modifications as compared to the parent binders (mixed or merged repeat modules).

Eight 2-in-1 DARPins from the 29 exhibited binding to the VEGF and α-CD3 DARPin targets and were selected for further characterisation as described in the following paragraphs. Of said eight 2-in-1 DARPins, five are N3C domains (i.e. 2-in-1 DARPins 11, 12, 13, 15 and 17 corresponding to SEQ ID NOs: 6, 7, 8, 9 and 10, respectfully) shown in FIG. 5A and three are N4C domains (i.e. 2-in-1 DARPins 18, 19 and 20, corresponding to SEQ ID NOs: 11, 12, and 13, respectfully) shown in FIG. 5B. Residues involved in the binding of the DARPin to its target(s) are known or identifiable by the person skilled in the art and together form a paratope (or define a binding surface) on the DARPin. In the 2-in-1 DARPins of the invention, a residue is marked as consensus if it occupies the same position within a paratope of a 2-in-1 DARPin as compared to the paratope of the two parent DARPins (i.e the DAPRins from which the 2-in-1 DARPin is combined). In this case, consensus residues can be determined by aligning the 2-in-1 DARPin sequences to the parental VEGF-binder and α-CD3 DARPin-blocker and compare the paratope positions as seen from the N-terminus of the VEGF-binder or the C-terminus of the α-CD3 DARPin-blocker (e.g. position N_5 is a consensus residue between the VEGF-binder and the α-CD3 DARPin-blocker for N3C 2-in-1 DARPins 11, 12, 13, 15 and 17, but not for N4C 2-in-1 DARPins 18, 19 and 20, since the N-cap of the α-CD3 DARPin-blocker then aligns with the internal repeat 1 of said N4C 2-in-1 DARPin). In FIGS. 5A-5B, the paratope residues comprised in a 2-in-1 DARPin are further highlighted according to their origin, i.e. in black (from α-CD3 DARPin blocker) or white (from VEGF binder), and said paratope positions are also indicated in the repeat numbering row (numbers boxed with black borders).

In order to facilitate the design of 2-in-1 DARPins, especially regarding the generation of mixed repeat modules, a 2D-representation method was developed. FIG. 5C shows an exemplary cartoon view of a N3C DARPin (i.e. a DARPin with N-cap, three internal repeats and a C-cap). The positions that make up the binding surface of the DARPin are labelled (N-terminal cap, C-terminal cap or internal repeat, numbering per repeat). The orientation of these residues allows to organize them in a 2D representation, as shown in FIG. 5D (dotted boxes in the 3D-view (5C) and 2D-view (5D) highlight the same stretch of residues). FIG. 5D accordingly shows, for each of the repeat modules, which positions of the paratope comprise framework residues or potential target interaction residues. This position map can be further extended to repeat domains comprising additional internal repeat domains, e.g. an N4C repeat domain would comprise an additional “Repeat 4” module with the same position map as for the repeats in the N3C. This 2D representation thus provides for an improved visualization of the 2-in-1 DARPins. FIG. 5E shows the 2D representation of the VEGF binder, the α-CD3 DARPin-blocker and the eight 2-in-1 DARPin designs. The coloring of the residues is as defined in FIGS. 5A-5B.

### High Level and Soluble Expression of Designed 2-In-1 DARPins

The 29 different 2-in-1 DARPin designs were expressed in E. coli cells and purified using their His-tag according to standard protocols. 5 ml of stationary overnight cultures (TB, 1% glucose, 50 mg/l of ampicillin; 37° C.) were used to inoculate 150 ml cultures (TB, 50 mg/l ampicillin, 37° C.). At an absorbance of 1.0 to 1.5 at 600 nm, the cultures were induced with 1 mM IPTG and incubated at 37° C. for 4-5 h while shaking. The cultures were centrifuged, and the resulting pellets were re-suspended in 10 ml of TBS₅₀₀ (50 mM Tris-HCl, 500 mM NaCl, pH 8) and stored at −20° C., before they were thawed, mixed with 50 KU DNase/ml and 1 mg/ml of lyzozyme and lysed (sonication or French press). Following the lysis, the samples were centrifuged and the supernatant was collected and filtrated. Imidazole (20 mM final concentration) was added to the homogenate. Proteins were purified over a bench-top Ni-nitrilotriacetic (Ni-NTA) acid column followed by a desalting column (NAP-25) step according to standard protocols and resins known to the person skilled in the art. Highly soluble 2-in-1 ankyrin repeat proteins were purified from E. coli culture (up to 200 mg ankyrin repeat protein per litre of culture) with a purity>95% as estimated from 4-12% SDS-PAGE. These non-formatted (or mono-domain) 2-in-1 DARPins were characterized biophysically by size exclusion chromatography, ProteOn surface plasmon resonance (SPR) target affinity assessment, ELISA, target protein-competition HTRF experiments, and/or SDS-PAGE.

### Example 2: SPR Binding Assays of 2-In-1 DARPins

An important feature of a 2-in-1 DARPin moiety of the invention is its affinity towards its first and second targets, in this exemplary design the VEGF molecule and the α-CD3 DARPin. Even more importantly, the binding of the 2-in-1 DARPin to the immobilized α-CD3 DARPin target should be impaired in presence of soluble VEGF to allow a switching mechanism.

Surface plasmon resonance (SPR) assays were used to determine the binding affinity and off-rate of 2-in-1 DARPins to the CD3 binding domain which can typically be comprised in a TCE molecule such as SEQ ID NO: 31.

All SPR data were generated using a Bio-Rad ProteOn XPR36 instrument with PBS-T (0.005% Tween 20) as running buffer. A new neutravidin sensor chip (NLC) was air-initialized and conditioned according to Bio-Rad manual.

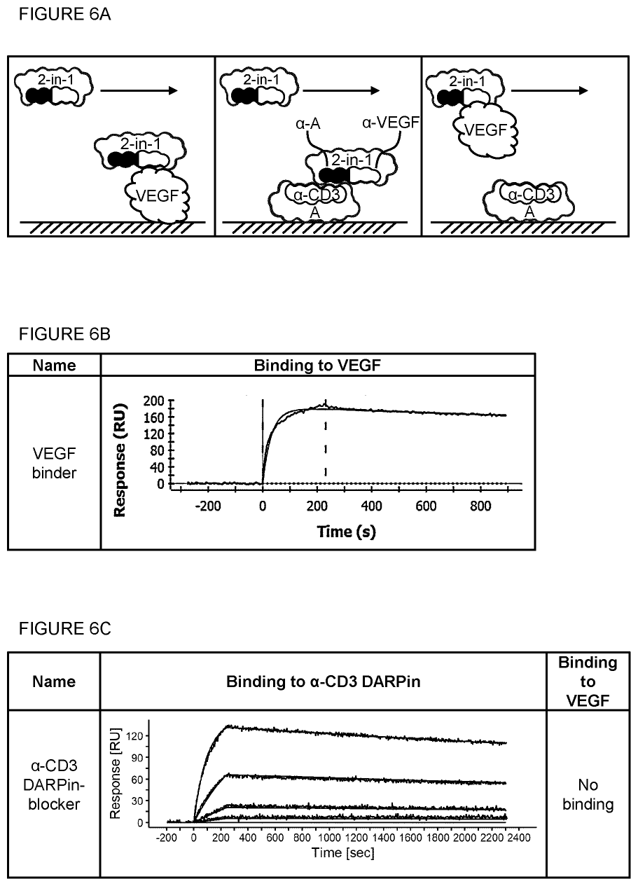

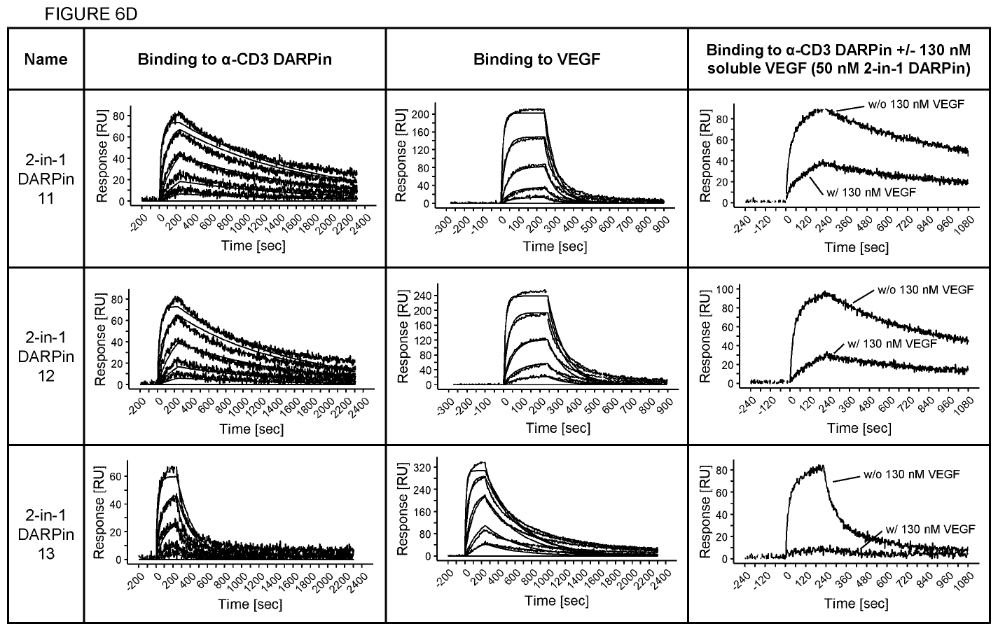

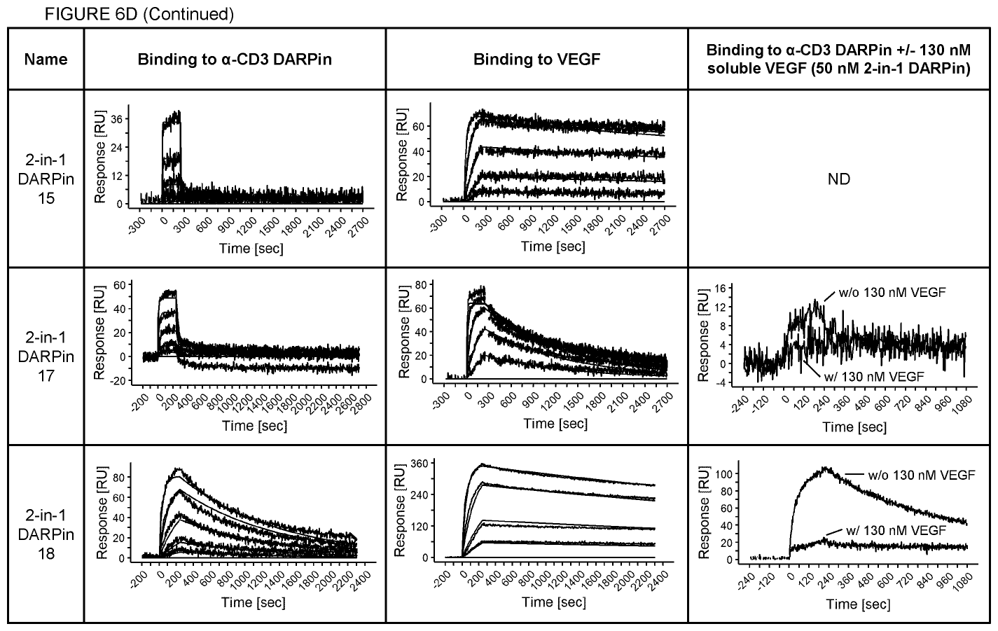

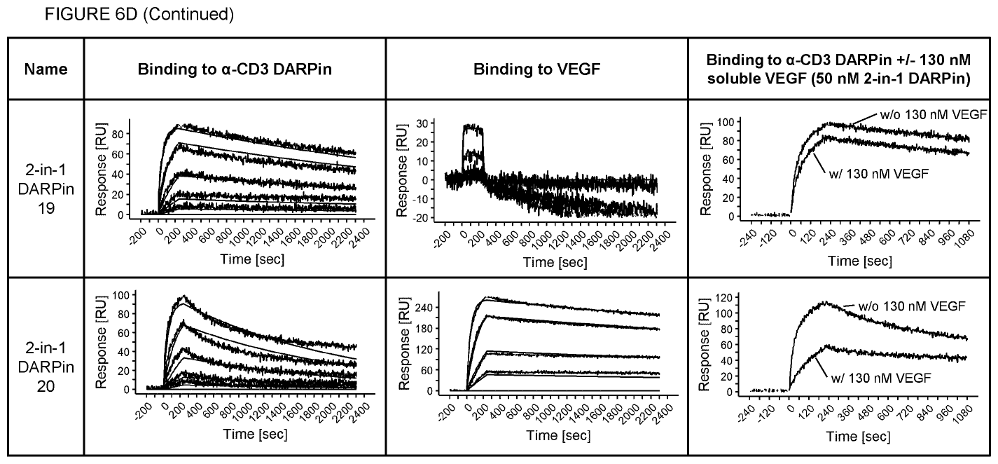

Eight 2-in-1 DARPin candidates of the 29 initially generated designs exhibited binding to VEGF covering a range of affinities, while 19 out of 29 constructs exhibited binding to the CD3-binding domain. From the 29 constructs, 8 constructs exhibited binding to both VEGF and α-CD3 DARPin (2-in-1 DARPins 11, 12, 13, 15, 17, 18, 19 and 20, corresponding to SEQ ID NOs 6 to 13 respectively). SPR data were also generated for the parental VEGF binder of SEQ ID NO: 1 and α-CD3 DARPin-blocker of SEQ ID NO: 2. Binding was tested against biotinylated CD3-specific binding domains having SEQ ID NO: 3 and against chip-immobilized VEGF₁₆₅ (FIG. 6A). The data were generated at 25° C. and with 5 binder concentration of 50 nM, 12.5 nM, 3.12 nM, 0.781 nM and 0.195 nM (i.e. multi-trace) except for the parental VEGF binder which was measured at a single concentration of 50 nM (single-trace) only. The SPR traces obtained with both parental binders of SEQ ID NOs: 1 and 2 are shown in FIG. 6B-6C. 2-in-1 DARPins of SEQ ID NOS: 6 to 13 exhibited binding to both VEGF and α-CD3 DARPin, with different affinities as can be observed in FIG. 6D (columns 1 and 2). Results are provided in Table 1 below.

Further SPR data were generated for 2-in-1 DARPins of SEQ ID NOs: 6 to 13, binding to biotinylated CD3-specific binding domains having SEQ ID NO: 3, on the chip. The data was generated at a single concentration of 50 nM, either in presence or absence of 130 nM VEGF (pre-incubated with the 2-in-1 DARPin). The right-most column in FIG. 6D shows the corresponding SPR traces).

TABLE 1

| | Target (Ligand) | | | | | | | | | | |
|---|---|---|---|---|---|---|---|---|---|---|---|
| | Biotinylated CD3-specific binding | | | | | | | | | | |
| | domain (Ligand1): SEQ ID NO: 3 | | | | | VEGF 165 (Ligand2) | | | | | K_{D} |
| | kon | koff | | | | kon | koff | | | | ratio |
| Construct | [×10⁵ M⁻¹ | [×10⁻³ | K_{D} | Rmax | Chi2 | [×10⁵ M⁻¹ | [×10⁻³ | KD | Rmax | Chi2 | (Ligand1/ |
| (Analyte) | s⁻¹] | s⁻¹] | [nM] | [RU] | [RU] | s⁻¹] | s⁻¹] | [nM] | [RU] | [RU] | Ligand2) |
| VEGF-binder | ND | 0.0 | ND | ND | ND | 6.46 | 0.14 | 0.21 | 180.0 | 25.0 | NA |
| (SEQ ID NO: 1) | | | | | | | | | | | |
| α-CD3 | 8.26 | 0.0892 | 0.11 | 144 | 4.2-7.9 | No | No | No | No | No | NA |
| DARPin - | | | | | | binding | binding | binding | binding | binding | |
| blocker (SEQ | | | | | | | | | | | |
| ID NO: 2) | | | | | | | | | | | |
| 2-in-1 DARPin | 6.91 | 0.67 | 0.96 | 74.95 | 11.91 | 13.80 | 15.30 | 11.10 | 246.6 | 31.3 | 8.65 × 10⁻² |
| 11 (SEQ ID | | | | | | | | | | | |
| NO: 6) | | | | | | | | | | | |
| 2-in-1 DARPin | 6.24 | 0.81 | 1.30 | 74.80 | 11.13 | 15.90 | 10.60 | 6.66 | 270.4 | 43.2 | 1.95 × 10⁻¹ |
| 12 (SEQ ID | | | | | | | | | | | |
| NO: 7) | | | | | | | | | | | |
| 2-in-1 DARPin | 6.93 | 8.00 | 11.50 | 94.62 | 15.09 | 32.90 | 2.68 | 0.81 | 52.7 | 6.20 | 14.2 |
| 13 (SEQ ID | | | | | | | | | | | |
| NO: 8) | | | | | | | | | | | |
| 2-in-1 DARPin | 1.51 | 205.0 | 1360.0 | 58.19 | 6.71 | 41.10 | 0.10 | 0.02 | 68.0 | 87.80 | 5.91 × 10⁴ |
| 15 (SEQ ID | | | | | | | | | | | |
| NO: 9) | | | | | | | | | | | |
| 2-in-1 DARPin | 5.99 | 23.50 | 392.0 | 58.63 | 29.27 | 46.30 | 0.85 | 0.18 | 65.0 | 8.50 | 2.18 × 10³ |
| 17 (SEQ ID | | | | | | | | | | | |
| NO: 10) | | | | | | | | | | | |
| 2-in-1 DARPin | 5.14 | 0.87 | 1.70 | 83.18 | 10.60 | 11.60 | 0.12 | 0.10 | 353.6 | ND | 17.0 |
| 18 (SEQ ID | | | | | | | | | | | |
| NO: 11) | | | | | | | | | | | |
| 2-in-1 DARPin | 4.50 | 0.20 | 0.44 | 86.27 | 14.58 | 13.80 | 94.90 | 68.50 | 64.3 | 4.60 | 6.47 × 10⁻³ |
| 19 (SEQ ID | | | | | | | | | | | |
| NO: 12) | | | | | | | | | | | |
| 2-in-1 DARPin | 3.50 | 0.50 | 1.44 | 94.30 | 16.23 | 12.90 | 0.09 | 0.69 | 263.2 | ND | 2.09 |
| 20 (SEQ ID | | | | | | | | | | | |
| NO: 13) | | | | | | | | | | | |

From these eight 2-in-1 DARPins, four were subsequently chosen to be formatted into multi-DARPin formats (also referred to as Switchdrug DARPins) to constitute switchable T-cell engager drugs including a TAA-binding domain and a CD3-binding domain as disclosed further below.

### Example 3: Switchdrug DARPins Formats Overview

Switchdrug DARPin constructs were cloned according to standard procedures into Format 1 ([α-CD3-binder]-[2-in-1 DARPin]), Format 3 ([2-in-1 DARPin]-[Ni2C]-[α-CD3 binder]), Format A ([α-TAA binder]-[α-CD3 binder]-[2-in-1 DARPin]) and Format B ([2-in-1 DARPin]-[α-TAA binder]-[α-CD3 binder]), with Ni2C being a non-binding DARPin (SEQ ID NO: 5) and α-TAA binder being an α-CD123 binding DARPin domain (SEQ ID NO: 4). In all these tested construct formats, an N-terminal His6-tag (SEQ ID NO: 34) was appended.

Switchdrug DARPins were expressed and purified following similar procedure as in Example 1, except that for constructs formatted in Format A and Format B, after IMAC purification, an additional purification step by a size exclusion chromatography on an ÄKTAxpress™ system was performed according to standard protocols and resins known to the person skilled in the art. Candidates were characterized biophysically by size exclusion chromatography and SDS-PAGE.

### Example 4: ELISA Assay to Assess Residual Binding of Switchdrug DARPins to VEGF

The residual binding of the following Switchdrug DARPin constructs to VEGF (biotinylated VEGF₁₆₅) was assessed:
Switchdrug DARPin 13-A (2-in-1 DARPin13 in Format A (SEQ ID NO: 23) with a His-tag (SEQ ID NO: 34) fused to its N-terminus),
Switchdrug DARPin 15-A (2-in-1 DARPin15 in Format A (SEQ ID NO: 24) with a His-tag (SEQ ID NO: 34) fused to its N-terminus),
Switchdrug DARPin 17-A (2-in-1 DARPin17 in Format A (SEQ ID NO: 25) with a His-tag (SEQ ID NO: 34) fused to its N-terminus),
Switchdrug DARPin 18-A (2-in-1 DARPin18 in Format A (SEQ ID NO: 26) with a His-tag (SEQ ID NO: 34) fused to its N-terminus),
Switchdrug DARPin 13-B (2-in-1 DARPin13 in Format B (SEQ ID NO: 27) with a His-tag (SEQ ID NO: 34) fused to its N-terminus),
Switchdrug DARPin 15-B (2-in-1 DARPin15 in Format B (SEQ ID NO: 28) with a His-tag (SEQ ID NO: 34) fused to its N-terminus),
Switchdrug DARPin 17-B (2-in-1 DARPin17 in Format B (SEQ ID NO: 29) with a His-tag (SEQ ID NO: 34) fused to its N-terminus),
Switchdrug DARPin 18-B (2-in-1 DARPin18 in Format B (SEQ ID NO: 30) with a His-tag (SEQ ID NO: 34) fused to its N-terminus).

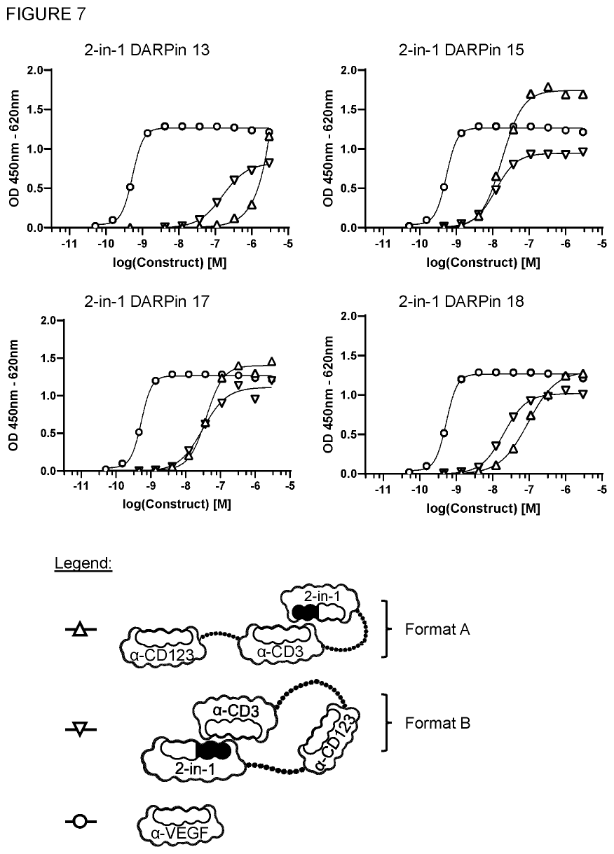

Binding of these constructs to VEGF was compared to the VEGF-binding domain of SEQ ID NO: 1 (FIG. 7).

Experimental conditions were as follows: nunc Maxisorp Plates were coated with 66 nM Neutravidin, washed, blocked with BSA and loaded with biotinylated VEGF₁₆₅ (VEGF) at 100 nM. Then a titration of the Switchdrug DARPins was applied, incubated and washed. Detection of bound his-tagged constructs was done by anti-RGS(his)4 mAb HRP conjugate, and detected by TMB substrate.

A significant difference in terms of EC50 of constructs in Format A or B compared to VEGF-binding domain can be observed. In general, the binding EC50 of the tested Switchdrug DARPins correlates well with the SPR data presented in FIG. 6D. Accordingly, these data show that the tested Switchdrug DARPins in closed form (i.e. without any soluble VEGF to compete with the binding of 2-in-1 DARPin to the α-CD3 DARPin) offer a concentration window which can be exploited for a switch mechanism.

### Example 5: ELISA Assay to Investigate Switch-Property of Switchdrug DARPins

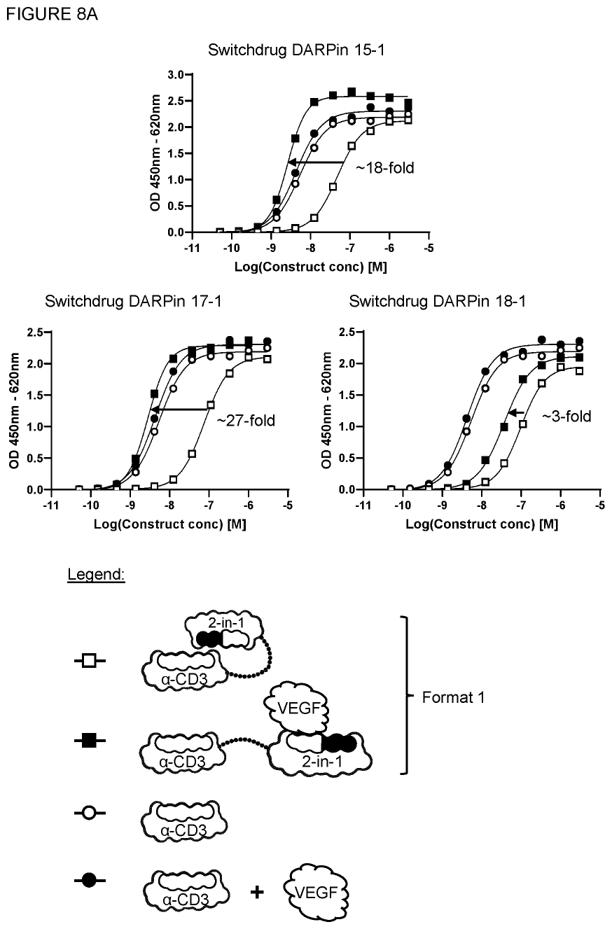

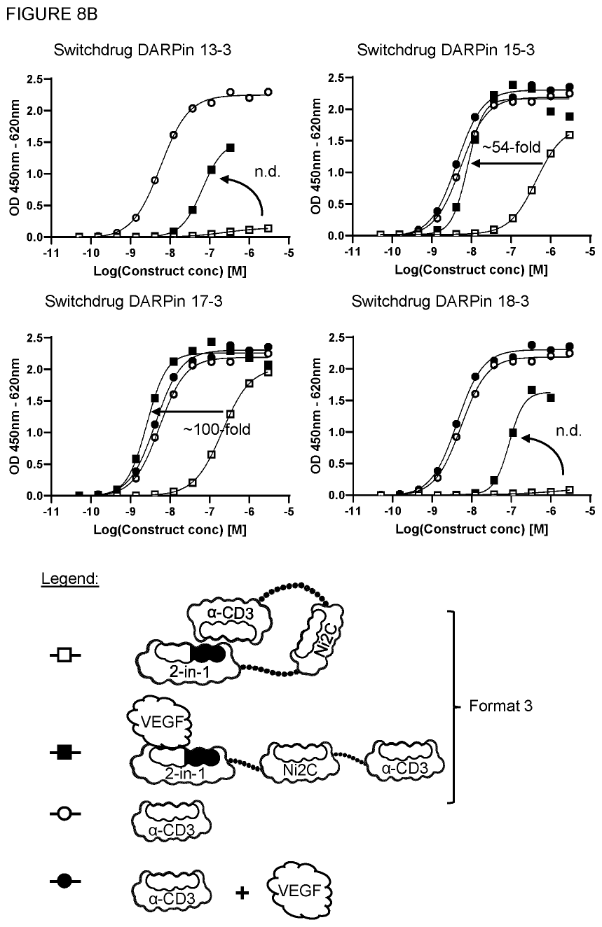

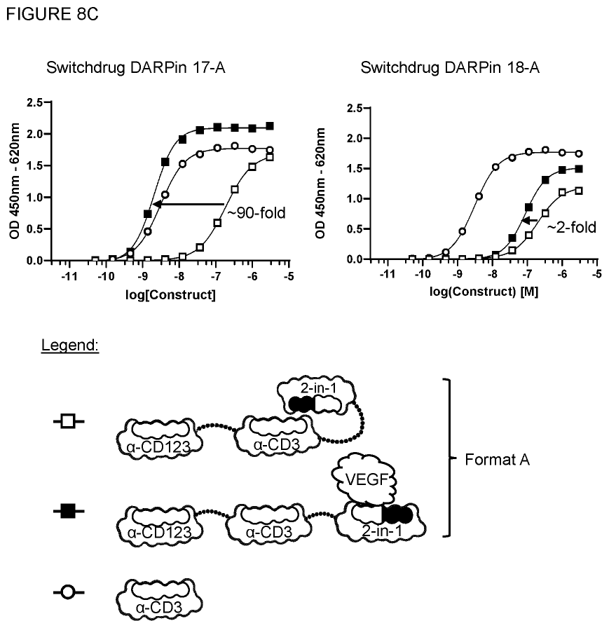

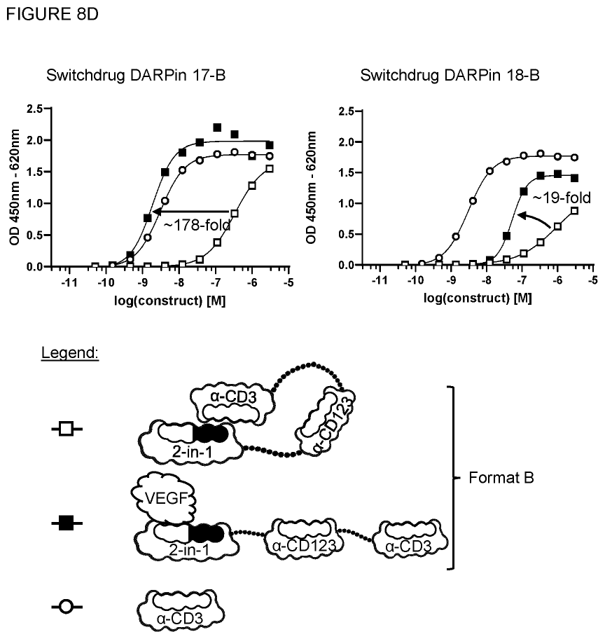

ELISA assays offer an easy and qualitative way to determine the binding of the Switchdrug DARPin constructs to a target. The difference in binding of Switchdrug DARPins to scCD3εγ in presence and absence of VEGF was assessed. Each of the 2-in-1 DARPin 13, 15, 17 or 18 was assembled into Switchdrug DARPins in Format 1, 3, A or B for titration experiments. Said Switchdrug DARPins were mixed with a constant concentration of VEGF (100 nM) and tested for binding to scCD3εγ. Monovalent CD3-binding domain (SEQ ID NO: 3) in presence and absence of 100 nM VEGF was used as positive control (FIGS. 8A-8D). The difference in EC50 of a construct in presence and absence of VEGF can be referred to as the “activation window”. Comparing FIGS. 8A and 8B, it seems that Format 3 is superior in terms of window of activation for the specific examples of Switchdrug DARPin constructs that can be compared.

Details of the tested Switchdrug DARPins (FIGS. 8A to 8D) are as follows:

FIG. 8A
Switchdrug DARPin 15-1 (2-in-1 DARPin15 in Format 1 (SEQ ID NO: 16) with a His-tag (SEQ ID NO: 34) fused to its N-terminus),
Switchdrug DARPin 17-1 (2-in-1 DARPin17 in Format 1 (SEQ ID NO: 17) with a His-tag (SEQ ID NO: 34) fused to its N-terminus),
Switchdrug DARPin 18-1 (2-in-1 DARPin18 in Format 1 (SEQ ID NO: 18) with a His-tag (SEQ ID NO: 34) fused to its N-terminus).

Format 1=[α-CD3 binder]-[2-in-1 DARPin].

FIG. 8B
Switchdrug DARPin 13-3 (2-in-1 DARPin13 in Format 3 (SEQ ID NO: 19) with a His-tag (SEQ ID NO: 34) fused to its N-terminus),
Switchdrug DARPin 15-3 (2-in-1 DARPin15 in Format 3 (SEQ ID NO: 20) with a His-tag (SEQ ID NO: 34) fused to its N-terminus),
Switchdrug DARPin 17-3 (2-in-1 DARPin17 in Format 3 (SEQ ID NO: 21) with a His-tag (SEQ ID NO: 34) fused to its N-terminus),
Switchdrug DARPin 18-3 (2-in-1 DARPin18 in Format 3 (SEQ ID NO: 22) with a His-tag (SEQ ID NO: 34) fused to its N-terminus),

Format 3=[2-in-1 DARPin]-[Ni2C]-[α-CD3 binder].

FIG. 8C
Switchdrug DARPin 17-A (2-in-1 DARPin17 in Format A (SEQ ID NO: 25) with a His-tag (SEQ ID NO: 34) fused to its N-terminus),
Switchdrug DARPin 18-A (2-in-1 DARPin18 in Format A (SEQ ID NO: 26) with a His-tag (SEQ ID NO: 34) fused to its N-terminus),

Format A=[α-CD123 binder]-[α-CD3 binder]-[2-in-1 DARPin].

FIG. 8D
Switchdrug DARPin 17-B (2-in-1 DARPin17 in Format B (SEQ ID NO: 29) with a His-tag (SEQ ID NO: 34) fused to its N-terminus),
Switchdrug DARPin 18-B (2-in-1 DARPin18 in Format B (SEQ ID NO: 30) with a His-tag (SEQ ID NO: 34) fused to its N-terminus),

Format B=[2-in-1 DARPin]-[α-CD123 binder]-[α-CD3 binder].

Switchdrug DARPin 13-3 and Switchdrug DARPin 18-3 show almost no binding in absence of VEGF, but robust binding to bscCD3εγ in presence of VEGF. In contrast, the EC50 of control CD3-binding domain (SEQ ID NO: 3) was not influenced by VEGF addition. Formats A and B (containing an α-CD123 binding domain instead of a non-binding Ni2C DARPin domain as in Formats 1 and 3) exhibited a similar behaviour as observed for Formats 1 and 3 (FIGS. 8C and 8D). Overall, Switchdrug DARPin 17-B offers the biggest activation window (˜178-fold) while at the same time it's binding can be fully restored to the CD3-binding domain control upon addition of 100 nM VEGF.

Experimental conditions were as follows: nunc Maxisorp Plates were coated with 66 nM Neutravidin, washed, blocked with BSA and loaded with biotinylated scCD3εγ at 20 nM. Switchdrug DARPins that were mixed with VEGF were titrated and pre-incubated for 1 day with VEGF in order to establish the thermodynamic equilibrium. These samples were loaded on the ELISA plate, incubated and washed, before the bound Switchdrug DARPins were detected as described above.

Accordingly, these data support that the presence or absence of 100 nM VEGF can give rise to an activation window for the binding of tested Switchdrug DARPins to their CD3 target.

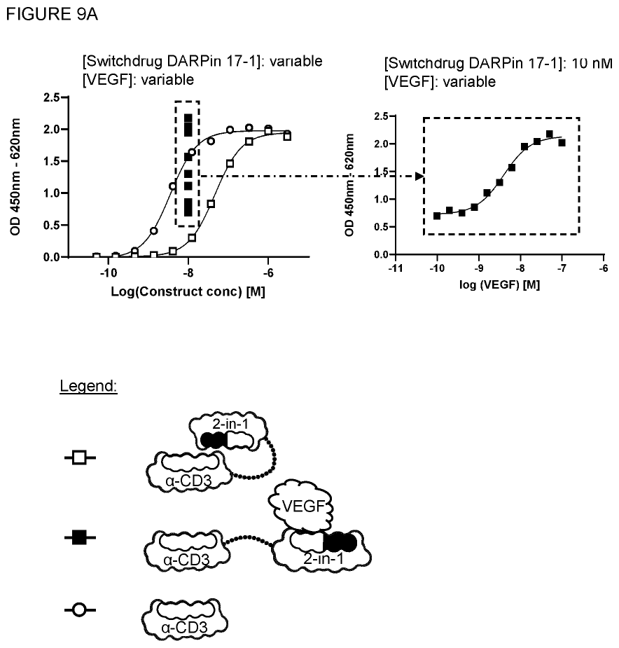

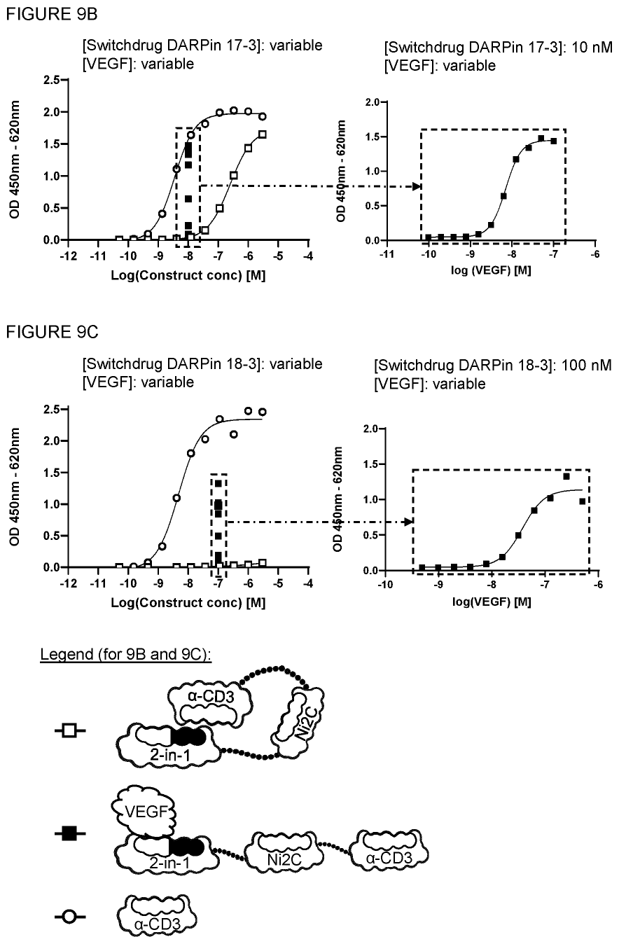

Next, the impact of VEGF concentration on the binding of the Switchdrug DARPins was assessed. Switchdrug DARPins 17-1, 17-3 and 18-3 as described above were kept at constant concentration while titrating the concentration of VEGF. FIG. 9A shows that Switchdrug DARPin 17-1 can regain binding to scCD3εγ dose-dependently when fixed at a concentration of 10 nM and incubating with titrated amounts of VEGF. The results for Switchdrug DARPins 17-3 and 18-3 are similarly represented in FIGS. 9B and 9C which show that Format 3 for the two constructs comprising 2-in-1 DARPin 17 is superior compared to the Format 1, as the constructs in absence of VEGF show no binding, but binding to CD3 can be re-gained in a VEGF dose-dependent way.

Accordingly, these data support that binding of exemplary Switchdrug DARPins to their target CD3 can be modulated by VEGF in a dose-dependent manner.

### Example 6: HTRF Assay as Validation

Homogeneous Time Resolved Fluorescence (HTRF) is a very fast and fully solution-based assay to determine protein interaction. To validate the results from the ELISA assay presented above, Switchdrug DARPins 13-3, 15-3, 17-3 or 18-3 were tested (details of these constructs are show in Example 5).

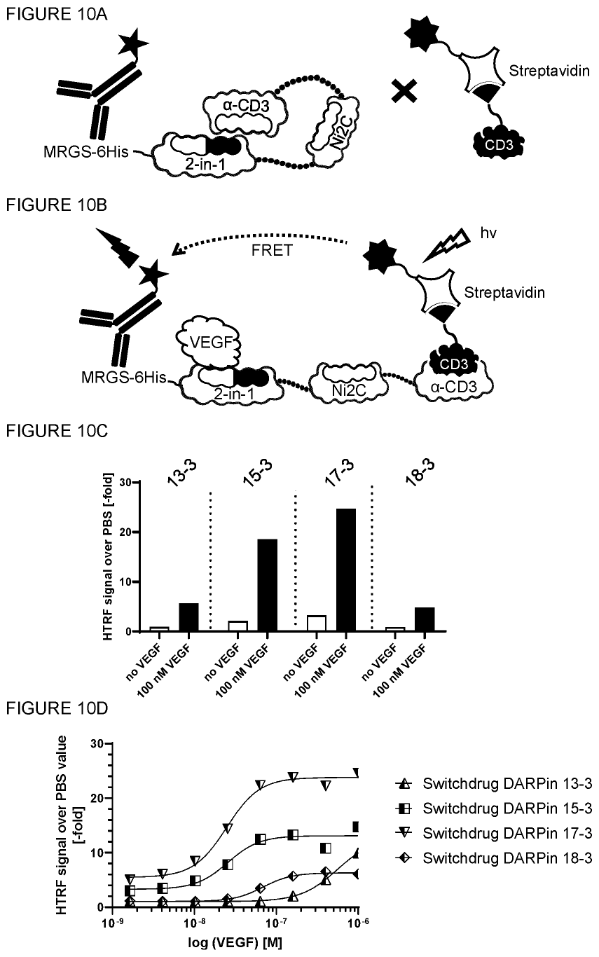

Said Switchdrug DARPins (20 nM final concentration) were mixed with 1 nM (final concentration) scCD3εγ, 1:200 (final concentration) of anti-FLAG-d2 HTRF antibody-FRET acceptor conjugate (Cisbio) and 1:200 (final concentration) of anti-strep-Tb antibody FRET donor conjugate (Cisbio, France). The principle of the assay in illustrated in FIGS. 10A-10B. If applicable, VEGF was added either at 100 nM (final concentration) (FIG. 10C) or titrated from 1 uM in 2.5-fold dilution steps (FIG. 10D). The mix was added to a well of a 384-well plate and incubated for 90 minutes. The HTRF was read-out on a Tecan M1000 using a 340 nm excitation wavelength and a 620±10 nm emission filter for background fluorescence detection and a 665±10 nm emission filter to detect the fluorescence signal for specific binding. The results shown in FIGS. 10C-10D confirm that activation windows for Switchdrug DARPins 13-3, 15-3, 17-3 or 18-3 can be observed similarly to the results of the ELISA assay in Example 5.

Accordingly, this data similarly supports that binding of Switchdrug DARPin TCEs to their target CD3 can be modulated by VEGF in a dose dependant manner.

### Example 7: Jurkat Cell Binding Assay

The following constructs were used in this assay:
Switchdrug DARPin 17-B (2-in-1 DARPin 17 in Format B (SEQ ID NO: 29) with a His-tag (SEQ ID NO: 34) fused to its N-terminus),

Format B control construct ([α-VEGF binder]-[α-CD123 binder]-[α-CD3 binder] of SEQ ID NO: 33 with a His-tag (SEQ ID NO: 34) fused to its N-terminus).

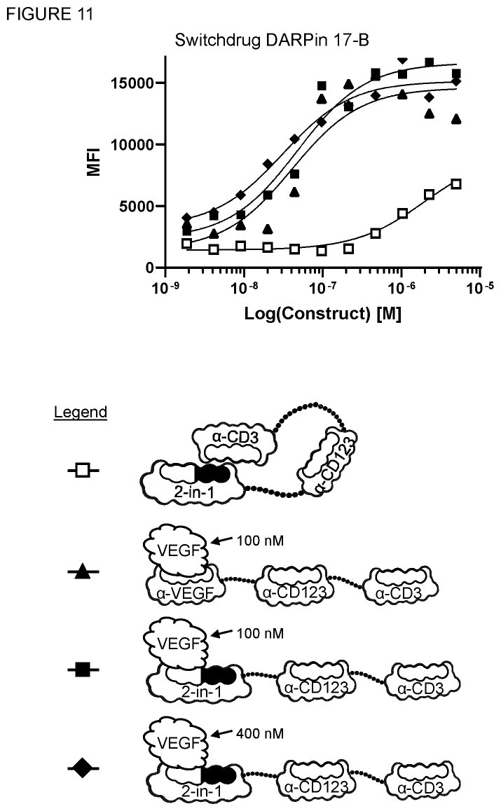

Binding of Switchdrug DARPin 17-B to human CD3 on Jurkat cells was measured by flow cytometry (Flow Cytometer FACS Canto). A titration of Switchdrug DARPin 17-B or Format B control construct in presence and absence of VEGF (100 nM or 400 nM) was incubated with 100′000 Jurkat cells per well for 60 min at 4° C. After washing, CD3 binding of the Switchdrug DARPin was detected by 1:100-diluted anti-penta-His antibody (30 min incubation at 4° C.). Cells were subsequently washed and stained for live-dead (aqua, 1:1000, Thermofisher) and resuspended in Cytofix fixation buffer (BD Biosciences). Median of mean fluorescence intensities of Alexa Fluor 488 binding on live cells were measured by Flow Cytometry and data was plotted using GraphPad Prism 8 (FIG. 11).

In absence of VEGF the Switchdrug DARPin 17-B shows only minimal binding at higher construct concentrations (>1 uM), whereas the addition of 100 nM VEGF reverts the binding to a similar level as the Format B control construct. Increasing the level of VEGF to 400 nM had no additional effect on Jurkat cell binding.

### Example 8: T-Cell Activation Assay

Specificity and potency of Switchdrug DARPins was assessed in an in vitro short-term T-cell activation assay by FACS measuring CD69 activation marker on CD8+ T-cells.

Details of the tested Switchdrug DARPins are as follows:

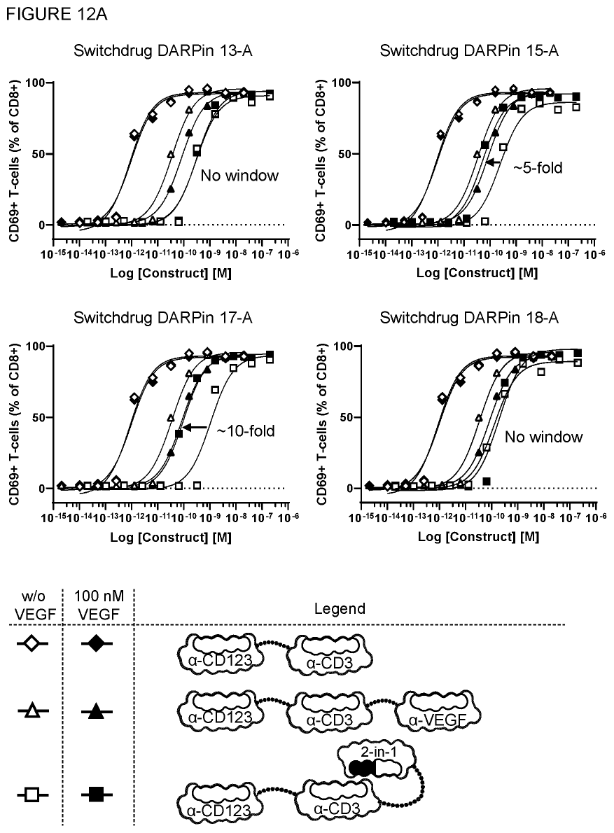

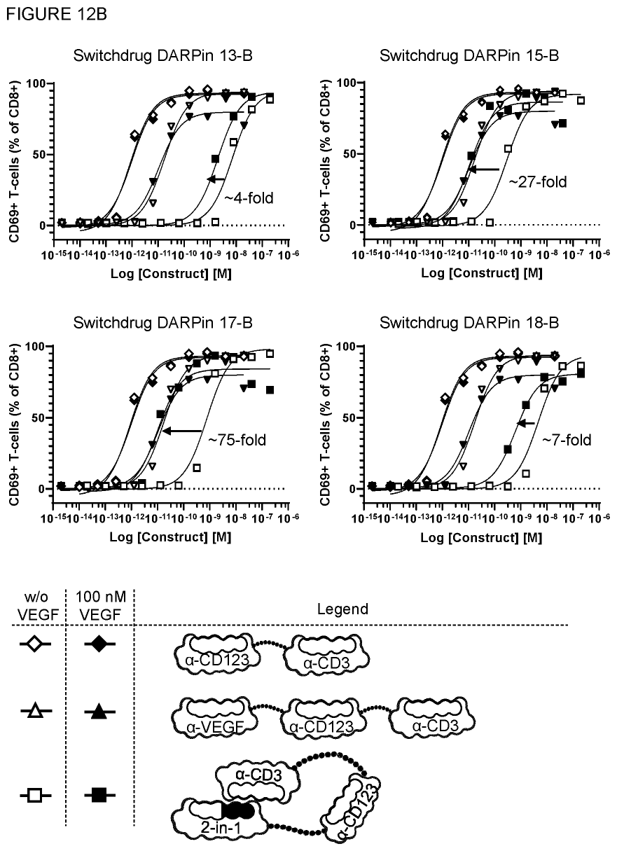

FIG. 12A:
Switchdrug DARPin 13-A (2-in-1 DARPin 13 in Format A (SEQ ID NO: 23) with a His-tag (SEQ ID NO: 34) fused to its N-terminus),
Switchdrug DARPin 15-A (2-in-1 DARPin 15 in Format A (SEQ ID NO: 24) with a His-tag (SEQ ID NO: 34) fused to its N-terminus),
Switchdrug DARPin 17-A (2-in-1 DARPin 17 in Format A (SEQ ID NO: 25) with a His-tag (SEQ ID NO: 34) fused to its N-terminus),
Switchdrug DARPin 18-A (2-in-1 DARPin 18 in Format A (SEQ ID NO: 26) with a His-tag (SEQ ID NO: 34) fused to its N-terminus),

Format A=[α-CD123 binder]-[α-CD3 binder]-[2-in-1 DARPin].

### Controls:

Control TCE ([α-CD123 binder]-[α-CD3 binder] of SEQ ID NO: 31 with a His-tag (SEQ ID NO: 34) fused to its N-terminus),
Format A control construct ([α-CD123 binder]-[α-CD3 binder]-[α-VEGF binder] of SEQ ID NO: 32 with a His-tag (SEQ ID NO: 34) fused to its N-terminus).

FIG. 12B:
Switchdrug DARPin 13-B (2-in-1 DARPin 13 in Format B (SEQ ID NO: 27) with a His-tag (SEQ ID NO: 34) fused to its N-terminus),
Switchdrug DARPin 15-B (2-in-1 DARPin 15 in Format B (SEQ ID NO: 28) with a His-tag (SEQ ID NO: 34) fused to its N-terminus),
Switchdrug DARPin 17-B (2-in-1 DARPin 17 in Format B (SEQ ID NO: 29) with a His-tag (SEQ ID NO: 34) fused to its N-terminus),
Switchdrug DARPin 18-B (2-in-1 DARPin 18 in Format B (SEQ ID NO: 30) with a His-tag (SEQ ID NO: 34) fused to its N-terminus),

Format B=[2-in-1 DARPin]-[α-CD123 binder]-[α-CD3 binder].

### Controls:

Control TCE ([α-CD123 binder]-[α-CD3 binder] of SEQ ID NO: 31 with a His-tag (SEQ ID NO: 34) fused to its N-terminus),
Format B control construct ([α-VEGF binder]-[α-CD123 binder]-[α-CD3 binder] of SEQ ID NO: 33 with a His-tag (SEQ ID NO: 34) fused to its N-terminus).

20′000 target tumor cells (Molm-13 cells, expressing CD123) and 100′000 effector T-cells (pan T-cells from healthy blood donors) were co-incubated (effector to target cell ratio of 5:1) with serial dilutions of Switchdrug DARPin samples in presence of 600 uM human serum albumin for 48 hours at 37° C. All Switchdrug DARPin samples and controls were pre-incubated with or without VEGF for at least 48 h at +4° C. After 48 h, cells were washed and stained with 1:5′000 Live/Dead FITC (Thermo Fisher), 1:250 mouse anti-human CD8 Pacific Blue (BD), and 1:250 mouse anti-hCD69-APC from BioLegend (BioLegend, US) antibodies for 30 min at 4° C. After washing and fixation, cells were analyzed on a Flow Cytometer Attune Nxt. T-cell activation was assessed by measuring CD69-positive cells on Live/Dead-negative and CD8-positive gated T-cells. FACS data was analyzed using FlowJo software and data was plotted using GraphPad Prism 8. Assays were performed two times with pan T-cells from two individual human donors. Switchdrug DARPins 13-A, 13-B, 15-A, 15-B, 17-A, 17-B, 18-A and 18-B were tested and compared to Control TCE, and respective Format A or B control constructs as represented in FIGS. 12A-12B. For both formats, Switchdrug DARPins comprising 2-in-1 DARPin 17 showed the best activation window (i.e. the difference in EC50 in absence and presence of 100 nM VEGF), with the largest window of 75-fold observed for Switchdrug DARPin 17-B (FIG. 12B). The Format B control construct did not reach the potency of the control TCE, even in the absence of VEGF. Hence, the addition of a second binding moiety to the N-terminus of the control TCE reduced the potency. Hence, the Switchdrug DARPins could maximally be expected to reach the potency of Format B control construct in presence of VEGF, which was the case for Switchdrug DARPin 15-B or 17-B. For Switchdrug DARPins 13-B or 18-B the addition of 100 nM VEGF was not sufficient to fully activate the construct.

### Example 9: Production and Characterisation of Additional 2-In-1 DARPins

Further 2-in-1 DARPins according to the invention have been produced and characterized. Unless specified otherwise, the experiments described in this section were essentially performed following procedures described in Examples 1 to 6.

### 9.1 Overview

Starting from four different target specific parent DARPins, 2-in-1 DARPins were designed and produced. The results are presented by means of five representative functional 2-in-1 DARPins. Based on the parent DARPins, these 2-in-1 DARPins can be classified in two different families, as detailed in Tables 2 and 3:

TABLE 2
Parent DARPins (mono-specific):

| Parent No | SEQ ID NO | Binding target |
|---|---|---|
| 1 | 86 | Maltose binding protein (MBP) |
| 2 | 87 | Aminoglycoside phosphotransferase (APH) |
| 3 | 3 | scCD3εγ |
| 4 | 88 | TargetA |

TABLE 3
2-in-1 DARPins (dual-specific):

| 2-in-1 | | SEQ | | |
|---|---|---|---|---|
| DARPin | Family | ID NO | Origin | Binding targets |
| 21 | 1 | 90 | Parent Nos 1 & 2 | APH & MBP |
| 22 | 1 | 91 | Parent Nos 1 & 2 | APH & MBP |
| 23 | 2 | 92 | Parent Nos 3 & 4 | TargetA & scCD3εγ |
| 24 | 2 | 93 | Parent Nos 3 & 4 | TargetA & scCD3εγ |
| 25 | 2 | 94 | Parent Nos 3 & 4 | TargetA & scCD3εγ |

Generally applicable processes are described here below in section 9.1, which may optionally be used to facilitate the design and characterisation of designed repeat domains of the invention, such as 2-in-1 DARPins.

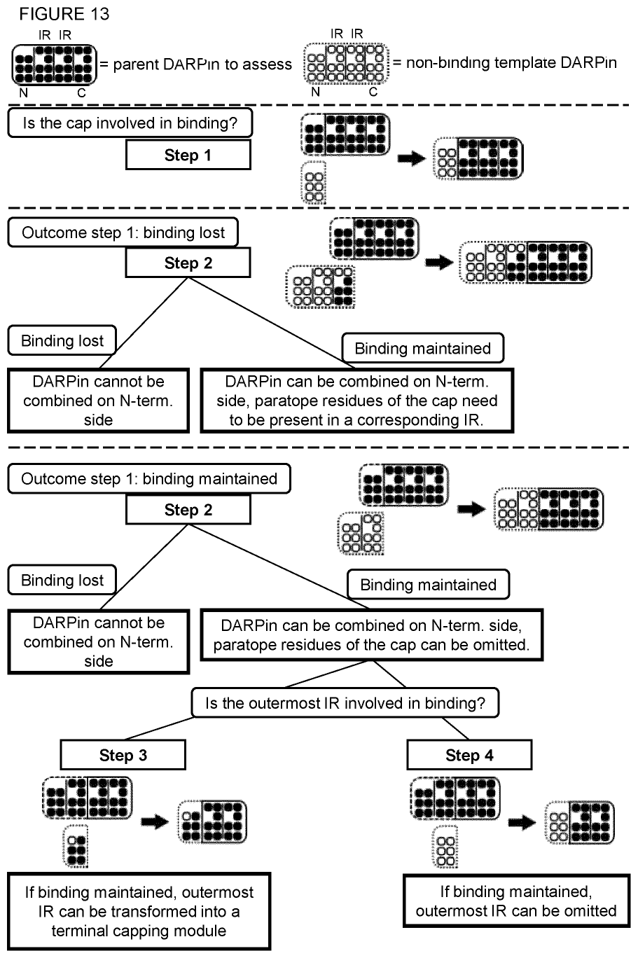

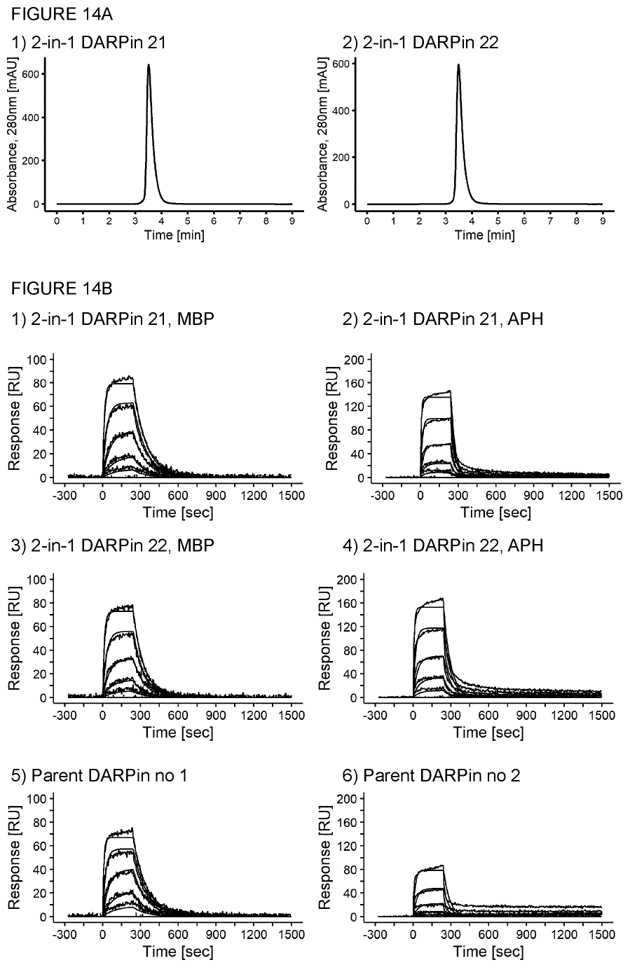

For the purpose of facilitating the design of designed repeat domains of the invention, one parent repeat domain or both parent repeat domains can be subjected to a profiling to determine if a parent repeat domain is compatible with being joined with another parent repeat domain (or a fragment thereof) at the N-terminal side and/or the C-terminal side to form a designed repeat domain with dual binding specificity, wherein binding to the two targets is mutually exclusive. Such a profiling provides an optimal starting point for subsequent combination of the parent repeat domains (or fragments thereof). This profiling comprises a series of systematic assays in which variants of a parent repeat domain of interest are created and evaluated. For each of the created variants, the ability to bind its target is tested to determine whether the N-terminal capping module, the C-terminal capping module, and/or internal repeat module(s) adjacent to the N-terminal capping module or the C-terminal capping module are important for target binding. This iterative binding assessment can be performed e.g. by ELISA. An exemplary threshold value for categorizing a variant as a variant that has lost target binding or a variant that has maintained target binding, as further described below, is an EC50 of approximatively 300 nM (i.e. higher EC50 values mean that the variant has lost binding, while lower EC50 values mean that the variant has maintained binding). Such a profiling of a parent repeat domain may include one or more steps, which are further described below (Steps 1 to 4) and also illustrated in FIG. 13 for a parent DARPin. FIG. 13 shows only the steps for the assessment of the N-terminal side of a parent DARPin. The steps for the assessment of the C-terminal side of a parent DARPin are analogous and follow the same logic. The steps described below and illustrated in FIG. 13 are exemplary and are not meant to be limiting.

Step 1. A terminal capping module (i.e. either the N-terminal capping module or the C-terminal capping module) of a parent repeat domain is replaced with a corresponding template non-binding terminal capping module (such as, e.g., SEQ ID NO: 36 or 38), and the resulting variant is tested for whether target binding is maintained or lost. This allows to determine whether or not the terminal capping module of the parent repeat domain is involved in target binding.
if target binding is lost, the terminal capping module is involved in target binding and step 2 (i.e. the version of step 2 for cases where binding was lost in step 1) may be performed;
if target binding is maintained, the terminal capping module is not involved in target binding and step 2 (i.e. the version of step 2 for cases where binding was maintained in step 1) may be performed.

This step 1 (and subsequently below steps 2, 3, and/or 4) may be performed separately for each terminal side of a parent DARPin of interest.

Step 2 (outcome of step 1 is lost target binding). The terminal capping module of a parent repeat domain that was replaced in step 1 is now replaced with a group of three connected repeat modules, the group comprising (from internal towards capping module) (1) a template non-binding internal repeat module (such as, e.g., SEQ ID NO: 89) in which the paratope residues of the replaced capping module are introduced, (2) a template non-binding internal repeat module, and (3) a template non-binding terminal capping module, and the resulting variant is tested for whether target binding is maintained or lost. Alternatively, said replacement can be done with a group of two connected repeat modules, the group comprising (from internal towards capping module) (1) a template non-binding internal repeat module in which the paratope residues of the replaced capping module are introduced, and (2) a template non-binding terminal capping module. This allows to determine whether or not target binding can be maintained if the paratope residues of the terminal capping module of the parent repeat domain are present in a designed internal repeat module that takes the place of the capping module in a designed repeat domain.
if target binding is lost, the considered terminal capping module and its paratope residues are essential for target binding, and the parent repeat domain cannot be joined with another repeat domain on this terminal side;
if target binding is maintained, the considered terminal side of the parent repeat domain is compatible with being joined with another (compatible) parent repeat domain. To do so, the considered terminal capping module can be removed and the paratope residues of said terminal capping module must be present in the adjacent internal repeat module that takes the place of the capping module within a designed repeat domain (this internal repeat module is derived from said second parent repeat domain or a newly designed internal repeat module).

Step 2 (outcome of step 1 is maintained target binding). The terminal capping module of a parent repeat domain that was replaced in step 1 is now replaced with a group of two connected repeat modules, the group comprising (from internal towards capping module) (1) a template non-binding internal repeat module and (2) a template non-binding terminal capping module, and the resulting variant is tested for whether target binding is maintained or lost. This allows to determine whether or not target binding can be maintained if the terminal capping module of the parent repeat domain is removed and the adjacent internal repeat module is combined with an internal repeat module of another repeat domain.
if target binding is lost, the parent repeat domain cannot be joined with another repeat domain on the considered terminal side (extension with additional internal repeat module(s) impairs binding);
if target binding is maintained, the parent repeat domain is compatible with being joined with another (compatible) parent repeat domain at the considered terminal side, and steps 3 and 4 may optionally be performed to further assess whether the outermost internal repeat module (i.e. the internal repeat module that is located adjacent to the considered terminal capping module) of the parent repeat domain is involved in target binding.

Step 3. Both the terminal capping module of a parent repeat domain that was replaced in step 1, and the adjacent internal repeat module, are now replaced with a template non-binding terminal capping module in which the paratope residues of the replaced internal repeat module are introduced, and the resulting variant is tested for whether target binding is maintained or lost. This allows to determine whether said adjacent internal repeat module must be kept to maintain target binding or whether it can be removed if its paratope residues are present in a terminal capping module that takes its place.
if target binding is lost, the considered adjacent internal repeat module is involved in target binding and should be kept as part of a designed repeat domain of the invention;
if target binding is maintained, the considered adjacent internal repeat module can be transformed into a terminal capping module (by having the paratope residues of said internal repeat module in the terminal capping module) in a designed repeat domain of the invention.

Step 4. Both the terminal capping module of a parent repeat domain that was replaced in step 1, and the adjacent internal repeat module, are now replaced with a template non-binding terminal capping module, and the resulting variant is tested for whether target binding is maintained or lost. This allows to determine whether or not target binding can be maintained if the terminal capping module and the adjacent internal repeat module of the parent repeat domain are removed and replaced with another repeat module.
if target binding is lost, the considered adjacent internal repeat module is involved in target binding and should be kept as part of a designed repeat domain of the invention.
if target binding is maintained, the considered adjacent internal repeat module is not involved in target binding and can be omitted in a designed repeat domain of the invention.

Respecting the order of the above steps is not strictly required, as more than one or all steps may be run in parallel by creating and assessing multiple or all variants in parallel. Furthermore, each of the above steps may be performed individually or in combination with one or more of the other steps to assess properties of a parent repeat domain.

The results of the assessment steps performed can be used to inform the design of designed repeat domains of the invention.

For the purpose of identifying and characterizing designed repeat domains of the invention, repeat domains that are created for the purpose of generating designed repeat domains of the invention are assessed for their target binding properties using techniques known in the art and/or as described herein. Properties of such generated repeat domains that are assessed for this purpose include the binding affinities to both targets and the mutual exclusive character of the binding to the first and second targets.

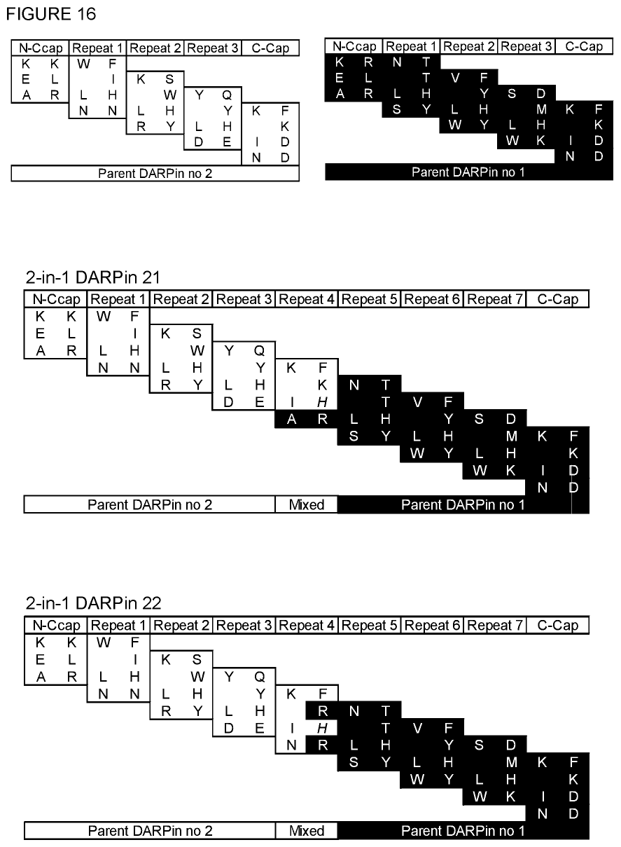

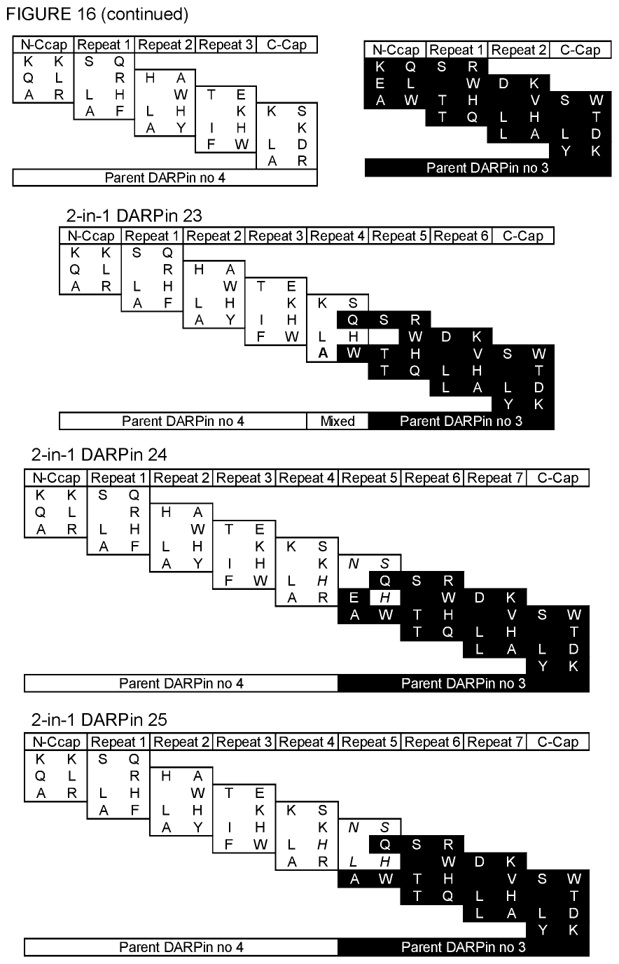

A 2D paratope representation of 2-in-1 DARPins described in this Example 9 and their respective parent DARPins is shown in FIG. 16.

### 9.2 Family 1

DARPin parent no 1 (binding to MBP) and parent no 2 (binding to APH) were described previously and correspond respectively to “off7” in Binz et al. 2004, (loc. Cit) and “AR 3b” in Amstutz, P, et al., 2005, Journal of Biological Chemistry 280.26:24715-24722. These DARPins were produced according to standard procedure known to the person skilled in the art.

The profiling method as described in section 9.1 was performed. The relevant information for parents no 1 and 2 is summarized in Table 4:

TABLE 4
Profiling of parents no 1 and 2.

| | | | | | | Can be | Can be |
|---|---|---|---|---|---|---|---|
| | | | | | | located at | located at |
| Parent | Repeat module analysis | | | | | N-term of a | C-term of a |
| no | N | IR1 | IR2 | IR3 | C | 2-in-1 DARPin | 2-in-1 DARPin |
| 1 | Z | Y | X | X | Z | Yes | Yes |
| 2 | X | X | X | Y | 7 | Yes | Yes, but not |
| | | | | | | | preferred |

where X means the repeat module cannot be modified or extended; Y means the repeat module type can be changed (e.g. transformed from an internal repeat module to a terminal capping module); Z means the repeat module can be removed; N is the N-terminal capping module; C is the C-terminal capping module; and IR1, IR2 and IR3 are the first, second and third internal repeat modules from the N-terminal end, respectively.

Based on this profiling, eleven 2-in-1 DARPin designs comprising the parent DARPin no 1 at the C-terminal side and the parent DARPin no 2 at the N-terminal side were created (second main step as referred in section 9.1). 2-in-1 DARPins comprising a range of between 5 (Table 5) and 10 (Table 6) internal repeat modules were created.

TABLE 5
Smallest design (overlapping paratopes).

| | Repeat modules alignment | | | | | | |
|---|---|---|---|---|---|---|---|
| N-term DARPin | N | IR1 | IR2 | IR3 | C | | |
| (parent 2) | | | | | | | |
| C-term DARPin | | | N | IR1 | IR2 | IR3 | C |
| (parent 1) | | | | | | | |
| 2-in-1 design | N | IR1 | IR2 | IR3/IR1 | IR2 | IR3 | C |

TABLE 6
Largest design (non-overlapping paratopes).

| | Repeat modules alignment | | | | | | | | | | | |
|---|---|---|---|---|---|---|---|---|---|---|---|---|
| N-term DARPin (parent 2) | N | IR1 | IR2 | IR3 | C | Rt | Rt | | | | | |
| C-term DARPin (parent 1) | | | | | | Rt | Rt | N | IR1 | IR2 | IR3 | C |
| 2-in-1 design | N | IR1 | IR2 | IR3 | C | Rt | Rt | N | IR1 | IR2 | IR3 | C |

In Tables 5 and 6, bold and underline typefaces are used to indicate the origin of the repeat modules; IR3/IR1 represents a mixed repeat module obtained from IR3 of parent 2 and IR1 of parent 1; and Rt represents a template internal repeat module (non-binding). An example of such template internal repeat module is SEQ ID NO: 89.

The characterization of these 2-in-1 designed repeat domains is shown for two representative constructs: 2-in-1 DARPin 21 (SEQ ID NO: 90) and 2-in-1 DARPin 22 (SEQ ID NO: 91), each of which has dual binding specificity for APH and MBP. Expression of the 2-in-1 DARPins was performed as described in Example 1.

### SEC Analysis

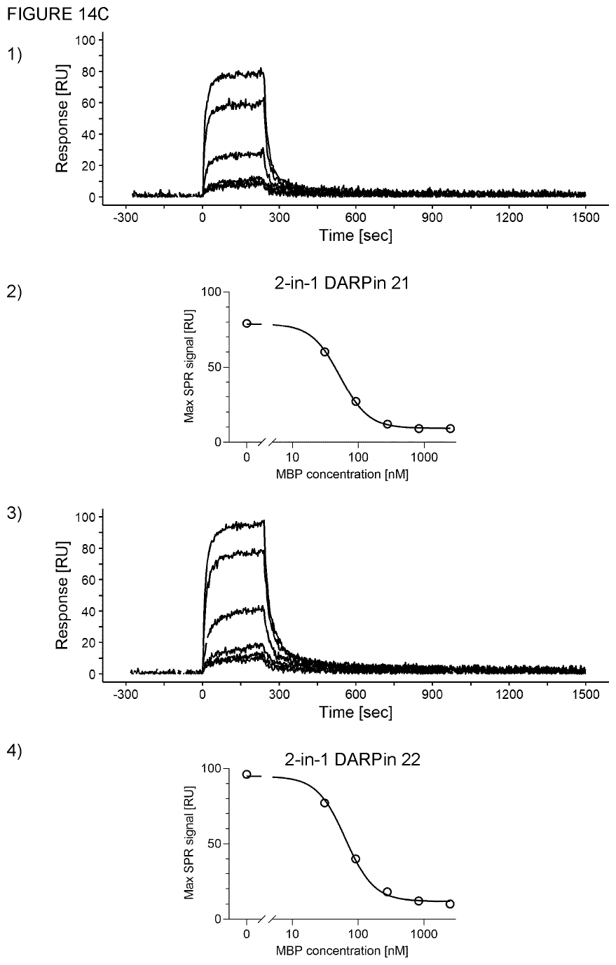

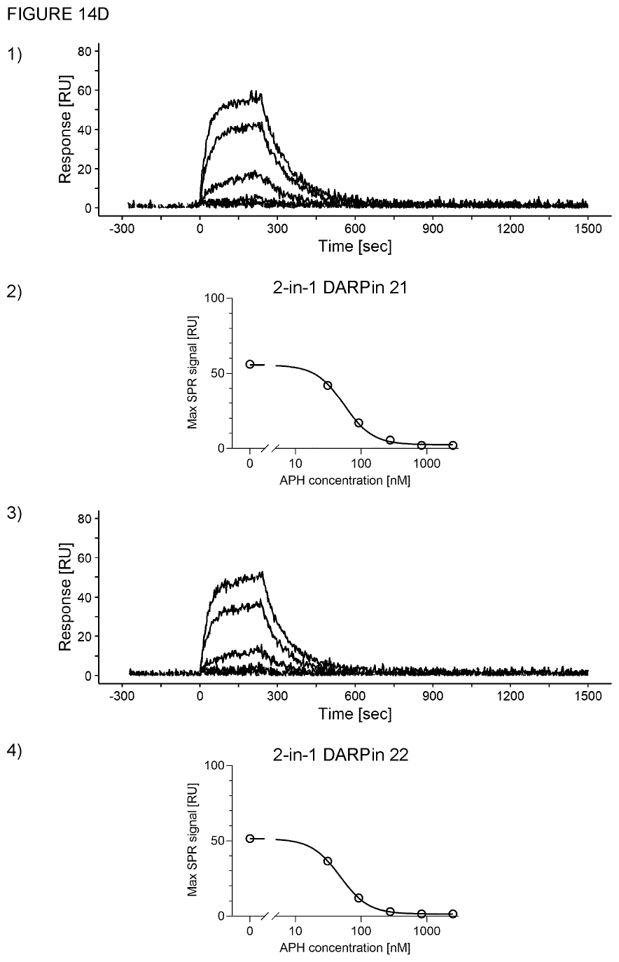

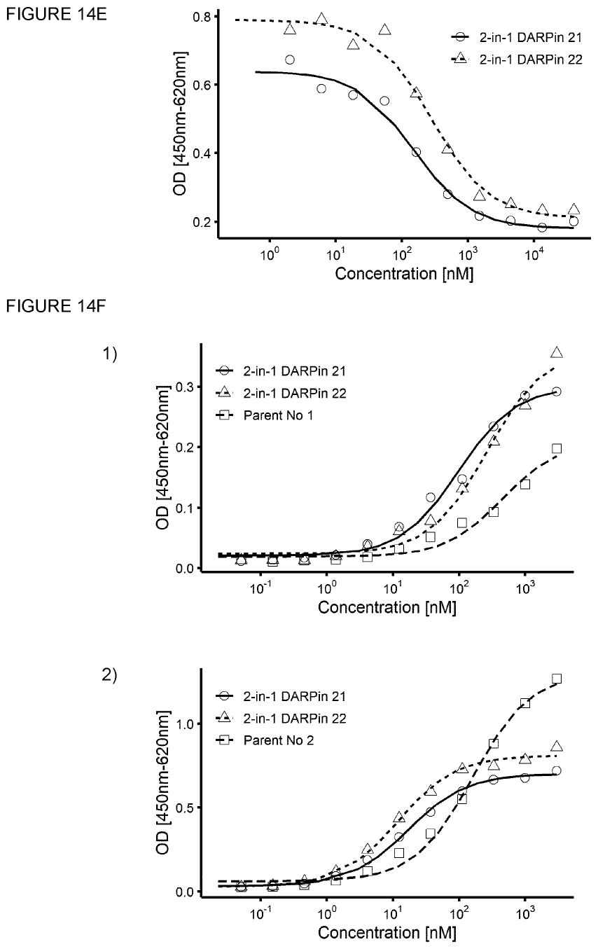

Samples (2 mg/ml) were analyzed on a GE Superdex 200 5/150 Increase GL column on an Agilent 1200 HPLC system in PBS at 0.5 ml/min flow rate. Results are shown in FIG. 14A.

### Binding Affinities (SPR)

Binding affinities of 2-in-1 DARPin 21 and 2-in-1 DARPin 22 to APH or MBP independently were determined by SPR analysis, as described in Example 2. Target molecules (APH or MBP) were coated on a HC200M chip (coating density: 550 RU for APH and 800 RU for MBP). The samples were diluted in ⅓ steps in PBS-T and run at 300 nM, 100 nM, 33.3 nM, 11.1 nM, 3.7 nM and 0 nM. SPR curves are shown in FIG. 14B, and K_{D} values in Table 7. Binding affinity of 2-in-1 DARPin 21 and 2-in-1 DARPin 22 to each of the targets are maintained compared to the respective affinities of the parent DARPins.

TABLE 7
Binding affinities

| | | |
|---|---|---|
| Sample | affinity to MBP, K_{D} | affinity to APH, K_{D} |

| | | | | |
|---|---|---|---|---|
| 2-in-1 DARPin 21 | 44.6 | nM | 66.9 | nM |
| 2-in-1 DARPin 22 | 53 | nM | 52.6 | nM |

| | | | |
|---|---|---|---|
| Parent no 2 | — | 147 | nM |

| | | | |
|---|---|---|---|
| Parent no 1 | 27.5 | nM | — |

Aminoglycoside phosphotransferase (APH) used in this assay corresponds to SEQ ID NO: 95, and maltose binding protein (MBP) used in this assay corresponds to SEQ ID NO: 96.

### Competitive Binding (SPR)

To assess the mutual binding exclusivity of 2-in-1 DARPin 21 and 2-in-1 DARPin 22 to APH and MBP, two competitive SPR assay were performed (Comp-SPR1 and Comp-SPR2).

In Comp-SPR1, APH was coated on a chip and 80 nM of 2-in-1 DARPins were pre-incubated with different concentration of soluble MBP (2500, 833, 277, 92, 31 or 0 nM) during 40 min. MBP acts as competitor for the binding of APH. The samples were then injected to the chip and the binding to APH was measured (FIG. 14C parts 1) and 3)). The response at the end of the injection (max SPR signal) was measured and plotted against the concentration of MBP (FIG. 14C parts 2) and 4)). FIG. 14C parts 1) and 2) show the results for 2-in-1 DARPin 21 and FIG. 14C parts 3) and 4) for 2-in-1 DARPin 22.

Comp-SPR2 corresponds to Comp-SPR1 in a reverse setting, i.e. in Comp-SPR2 MBP was coated on a chip and 80 nM of 2-in-1 DARPins were pre-incubated with different concentration (2500, 833, 277, 92, 31 or 0 nM) of soluble APH during 40 min. APH acts as competitor for the binding of MBP. The samples were then injected to the chip and the binding to MBP was measured (FIG. 14D parts 1) and 3)). The response at the end of the injection (max SPR signal) was measured and plotted against the concentration of APH (FIG. 14D parts 2) and 4)). FIG. 14D parts 1) and 2) show the results for 2-in-1 DARPin 21 and FIG. 14D parts 3 and 4) for 2-in-1 DARPin 22. The IC50 values resulting from the above two competitive SPR assays are show in Table 8:

TABLE 8
IC50 values

| | APH coated, MBP | MBP coated, APH |
|---|---|---|
| | competitor | competitor |
| Sample | (Comp-SPR1) | (Comp-SPR2) |
| 2-in-1 DARPin 21 | 53 nM | 55 nM |
| 2-in-1 DARPin 22 | 64 nM | 47 nM |

As shown by the drop in SPR signals at the end of the injections (max SPR signal), in each case an increasing concentration of competitor reduces the binding to the coated target. Hence the binding of the 2-in-1 DARPins 21 and 22 to the first and second targets (MBP and APH) is exclusive between each other.

### Competitive Binding (ELISA)

The mutual binding exclusivity of 2-in-1 DARPins of the invention can alternatively be measured by a competitive binding assay based on ELISA. A Nunc Maxisorp plate was first coated with APH. 2-in-1 DARPin 21 and 2-in-1 DARPin 22 at a final concentration of 100 nM where exposed to a ⅓ steps serial dilution of MBP (40000, 13333, 4444, 1481, 494, 165, 54.9, 18.3, 6.1, 2.0, or 0 nM) for 1 hour. A standard readout was subsequently performed. Results are sown in FIG. 14E. A dose response can be observed.

### Binding Affinities (ELISA)

Binding to target MBP or APH was tested separately for 2-in-1 DARPin 21 and 2-in-1 DARPin 22 in a standard ELISA assay. Parent DAPRins no 1 and 2 were used as positive controls. Assays were performed as described in Example 4. The results are shown in FIG. 14F, where plot 1) represents binding of the samples to MBP and plot 2) represent binding of the samples to APH.

### 9.3 Family 2

DARPin parent no 3 (binding to scCD3εγ) as well as the scCD3εγ target protein itself were described in WO2022129428. These DARPins were produced according to standard procedure known to the person skilled in the art.

The profiling method as described in section 9.1 was performed for both parent DARPins no 3 and no 4. The relevant information is summarized in Tables 9 and 10:

TABLE 9
Profiling of parent no 3.

| | | | | | Can be | Can be |
|---|---|---|---|---|---|---|
| | | | | | located at | located at |
| Parent | Repeat module analysis | | | | N-term of a | C-term of a |
| no | N | IR1 | IR2 | C | 2-in-1 DARPin | 2-in-1 DARPin |
| 3 | Y | X | X | X | Yes, but not | Yes |
| | | | | | preferred | |

TABLE 10
Profiling of parent no 4.

| | | | | | | Can be | Can be |
|---|---|---|---|---|---|---|---|
| | | | | | | located at | located at |
| Parent | Repeat module analysis | | | | | N-term of a | C-term of a |
| no | N | IR1 | IR2 | IR3 | C | 2-in-1 DARPin | 2-in-1 DARPin |
| 4 | X | X | X | X | Y | Yes | No |

In Tables 9 and 10, X means the repeat module cannot be modified or extended; Y means the repeat module type can be changed (e.g. transformed from an internal repeat module to a terminal capping module); N is the N-terminal capping module; C is the C-terminal capping module; and IR1, IR2 and IR3 are the first, second and third internal repeat modules from the N-terminal end, respectively.

Based on this profiling, ten 2-in-1 designed repeat domains comprising the parent DARPin no 3 (or part of it) at the C-terminal side and the parent DARPin no 4 (or part of it) at the N-terminal side were created (second main step as referred in section 9.1). 2-in-1 DARPins comprising a range of between 6 (Table 11) and 10 (Table 12) internal repeat modules were created.

TABLE 11
Smallest design (overlapping paratopes).

| | Repeat modules alignment | | | | | | | |
|---|---|---|---|---|---|---|---|---|
| N-term DARPin | N | IR1 | IR2 | IR3 | C | | | |
| (parent 4) | | | | | | | | |
| C-term DARPin | | | | | N | IR1 | IR2 | C |
| (parent 3) | | | | | | | | |
| 2-in-1 design | N | IR1 | IR2 | IR3 | C/N | IR1 | IR2 | C |

where bold and underline typefaces are used to indicate the origin of the repeat modules; and C/N represents a mixed repeat module obtained from C of parent 4 and N of parent 3.

TABLE 12
Largest design (non-overlapping paratopes).

| | Repeat modules alignment | | | | | | | | | | | |
|---|---|---|---|---|---|---|---|---|---|---|---|---|
| N-term DARPin | N | IR1 | IR2 | IR3 | C | Rt | Rt | Rt | | | | |
| (parent 4) | | | | | | | | | | | | |
| C-term DARPin | | | | | | Rt | Rt | Rt | N | IR1 | IR2 | C |
| (parent 3) | | | | | | | | | | | | |
| 2-in-1 design | N | IR1 | IR2 | IR3 | C | Rt | Rt | Rt | N | IR1 | IR2 | C |

where Rt represents a template internal repeat module (non-binding). An example of such template internal repeat module is SEQ ID NO: 89.

The characterization of these 2-in-1 designed repeat domains is shown for three representative constructs: 2-in-1 DARPin 23 (SEQ ID NO: 92), 2-in-1 DARPin 24 (SEQ ID NO: 93) and 2-in-1 DARPin 25 (SEQ ID NO: 94), which have dual binding specificities for scCD3εγ and TargetA. Expression of the 2-in-1 DARPins was performed as described in Example 1.

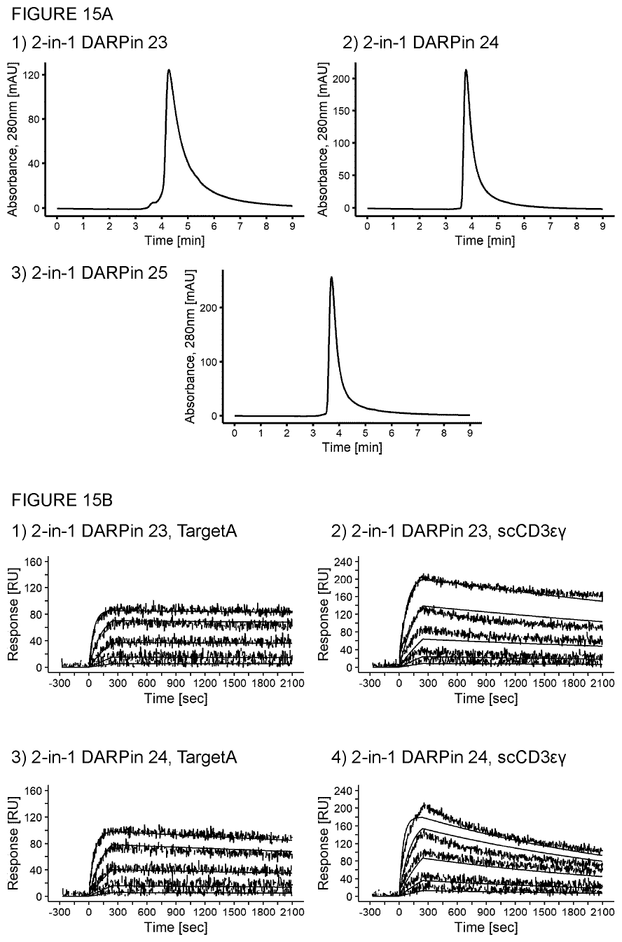

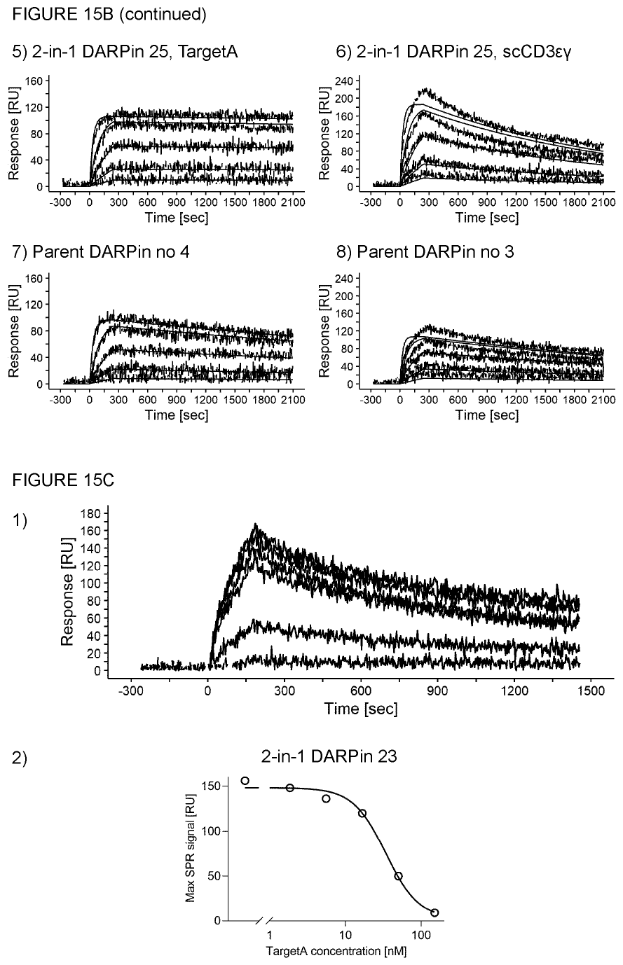

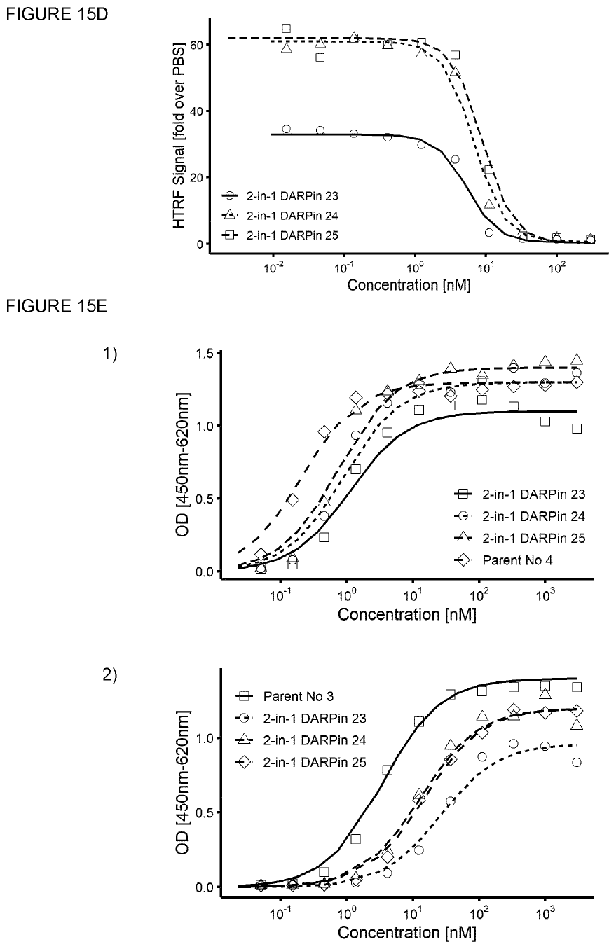

SEC analysis was performed as in section 9.2. Results are shown in FIG. 15A.

### Binding Affinities (SPR)

Binding affinities of 2-in-1 DARPin 23, 2-in-1 DARPin 24 and 2-in-1 DARPin 25 to scCD3εγ or TagretA independently were determined by SPR analysis, as described in Example 2. Biotinylated target molecules (bio-scCD3εγ or bio-TargetA) were coated on an NAD chip (coating density: 460 RU for bio-scCD3εγ and 560 RU for bio-TargetA). The samples were diluted in ⅓ steps in PBS-T and run at 333.33 nM, 111.11 nM, 37.00 nM, 12.34 nM, 4.11 nM and 0 nM. SPR curves are shown in FIG. 15B, and K_{D} values in Table 13. Binding affinity of 2-in-1 DARPin 23, 2-in-1 DARPin 24 and 2-in-1 DARPin 25 to each of the targets are maintained compared to the respective affinities of the parent DARPins.

TABLE 13
Binding affinities

| | affinity to bio- | affinity to bio- |
|---|---|---|
| Sample | scCD3εγ, K_{D} | TargetA, K_{D} |
| 2-in-1 DARPin 23 | 3.81 nM | 0.024 nM |
| 2-in-1 DARPin 24 | 4.67 nM | 0.119 nM |
| 2-in-1 DARPin 25 | 4.05 nM | 0.018 nM |
| Parent no 3 | 2.03 nM | — |
| Parent no 4 | — | 0.160 nM |

### Competitive Binding (SPR)

The mutual binding exclusivity of 2-in-1 DARPin 23 to scCD3εγ and TargetA was assessed with a competitive SPR assay (similar to Comp-SPR1 & Comp-SPR2 in section 9.2).

Biotinylated scCD3εγ was coated on a NAD chip and 50 nM of 2-in-1 DARPin was pre-incubated with different concentration of soluble TargetA (150, 50, 16.7, 5.5, 1.85 or 0 nM) for 4.5 hours. TargetA acts as competitor for the binding of scCD3εγ. The samples were then injected to the chip and the binding to scCD3εγ was measured (FIG. 15C part 1)). The response at the end of the injection (max SPR signal) was measured and plotted against the concentration of TargetA (FIG. 15C part 2)). The IC50 value resulting from the above competitive SPR assay is show in Table 14:

TABLE 14
IC50 values

| Sample | scCD3εγ coated, TargetA competitor |
|---|---|
| 2-in-1 DARPin 23 | 34 nM |

As shown by the drop in SPR signals at the end of the injections (max SPR signal), an increasing concentration of competitor reduces the binding to the coated target. Hence the binding of the 2-in-1 DARPins 23 to the first and second targets (scCD3εγ and TargetA) is exclusive between each other.

### Competitive Binding (HTRF)

The mutual binding exclusivity of 2-in-1 DARPins of the invention can alternatively be measured by a competitive binding assay based on HTRF. In this assay, binding of 2-in-1 DARPins to bio-scCD3εγ in presence of different concentrations of non-biotinylated TargetA competitor is assessed. The HTRF assay was essentially done as described in example 6. Following parameters have been used: 1 nM bio-scCD3εγ, 3 nM of 2-in-1 DARPin, titration of TargetA competitor at 300, 100, 33.333, 11.111, 3.704, 1.235, 0.412, 0.137, 0.046, 0.015 and 0 nM. The curve resulting from this HTRF assay are shown in FIG. 15D. Based on this data, it can be concluded that in presence of an increasing concentration of competitor (here TargetA), binding to the second target (here scCD3εγ) is hindered, hence binding of the 2-in-1 DARPin to the first or second target is mutually exclusive.

### Binding Affinities (ELISA)

Binding to target scCD3εγ or TargetA was tested separately for 2-in-1 DARPin 23, 2-in-1 DARPin 24 and 2-in-1 DARPin 25 in a standard ELISA assay. Parent DAPRins no 3 and 4 were used as positive controls. Assays were performed as described in Example 4. The results are shown in FIG. 15E, where plot 1) represents binding of the samples to TargetA and plot 2) represents binding of the samples to scCD3εγ.

In conclusion, the Examples demonstrate that designed repeat domains having a first binding specificity for a first target and a second binding specificity for a second target, wherein binding of said designed repeat domain to said first and second targets is mutually exclusive, can be generated using many different repeat domains with differing target specificities as parent repeat domains. Thus, the Examples provide a generally applicable method of generating such designed repeat domains and establish the general concept of designed repeat domains having such novel properties.

The specification is most thoroughly understood in light of the teachings of the references cited within the specification. The aspects within the specification provide an illustration of aspects of the invention and should not be construed to limit the scope of the invention. The skilled artisan readily recognizes that many other aspects are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific aspects of the invention described herein. Such equivalents are intended to be encompassed by the following claims.

1. A method of producing a designed repeat domain, the method comprising the steps of:
(i) providing a first repeat domain and a second repeat domain, wherein said first repeat domain has binding specificity for a first target and said second repeat domain has binding specificity for a second target; and
(ii) joining part of said first repeat domain and part of said second repeat domain to form a designed repeat domain with dual binding specificity, by covalently combining at least one repeat module of said first repeat domain and at least one repeat module of said second repeat domain;
wherein said designed repeat domain has binding specificity for said first target and binding specificity for said second target, and wherein binding of said designed repeat domain to said first and second targets is mutually exclusive.

2. The method of claim 1, wherein said covalently combining at least one repeat module of said first repeat domain and at least one repeat module of said second repeat domain comprises covalently connecting said at least one repeat module of said first repeat domain and said at least one repeat module of said second repeat domain.

3. The method of claim 2, wherein said at least one repeat module of said first repeat domain and said at least one repeat module of said second repeat domain are covalently connected by a linker, wherein said linker is preferably a peptide linker or a non-binding repeat module.

4. The method of claim 1, wherein said covalently combining at least one repeat module of said first repeat domain and at least one repeat module of said second repeat domain comprises covalently merging said at least one repeat module of said first repeat domain and said at least one repeat module of said second repeat domain.

5. The method of claim 1, wherein said designed repeat domain comprises an N-terminal capping module and/or a C-terminal capping module, and at least one internal repeat module.

6. The method of claim 1, wherein said designed repeat domain binds to said first target with a dissociation constant (K_{D}) below 10⁻⁵ M and/or wherein said designed repeat domain binds to said second target with a dissociation constant (K_{D}) below 10⁻⁵ M.

7. The method of claim 1, wherein said first repeat domain is an ankyrin repeat domain and said second repeat domain is an ankyrin repeat domain, and/or wherein said designed repeat domain is an ankyrin repeat domain.

8. A designed repeat domain obtainable by the method of claim 1.

9. A designed repeat domain having a first binding specificity for a first target and a second binding specificity for a second target, wherein binding of said designed repeat domain to said first and second targets is mutually exclusive.

10. The designed repeat domain of claim 9, wherein said designed repeat domain binds to said first target with a dissociation constant (K_{D}) below 10⁻⁵ M and/or wherein said designed repeat domain binds to said second target with a dissociation constant (K_{D}) below 10⁻⁵ M.

11. The designed repeat domain of claim 9, wherein said designed repeat domain comprises an N-terminal capping module and/or a C-terminal capping module, and at least one internal repeat module.

12. The designed repeat domain of claim 9, wherein the designed repeat domain is an ankyrin repeat domain.

13. The designed repeat domain of claim 9, wherein the designed repeat domain comprises at least one internal repeat module comprising a sequence selected from SEQ ID NOs: 75 to 81.

14. The designed repeat domain of claim 9, wherein said designed repeat domain comprises (i) an N-terminal capping module comprising a sequence selected from the group consisting of (1) SEQ ID NOs: 35, 36, 47 to 63, 83 to 85 and 97 to 100 and (2) sequences in which up to 9 amino acids in any of SEQ ID NOs: 35, 36, 47, 53, 59, 60, 83 and 84 are substituted by another amino acid; and/or (ii) a C-terminal capping module comprising a sequence selected from the group consisting of (1) SEQ ID NOs: 37, 38 and 64 to 74 and (2) sequences in which up to 9 amino acids in any of SEQ ID NOs: 37, 38 and 69 are substituted by another amino acid, and/or (iii) at least one internal repeat module comprising a sequence selected from the group consisting of (1) SEQ ID NOs: 40 to 46 and (2) sequences in which up to 9 amino acids in any of SEQ ID NOs: 40 to 46 are substituted by another amino acid.

15. A recombinant protein comprising the designed repeat domain of claim 9.

16. The protein according to claim 15, wherein the protein further comprises a therapeutic agent, wherein the therapeutic agent is preferably a binding molecule.

17. An isolated nucleic acid molecule encoding the designed repeat domain of claim 9 or a protein comprising the designed repeat domain.

18. A pharmaceutical composition comprising one or more of:
(i) the designed repeat domain of claim 9,
(ii) a recombinant protein comprising the designed repeat domain, and/or
(iii) a nucleic acid molecule encoding designed repeat domain of the protein,
and a pharmaceutically acceptable carrier or diluent.

19. A method of treating a medical condition, the method comprising the step of administering to a patient in need thereof a therapeutically effective amount of the designed repeat domain of claim 9, a recombinant protein comprising the designed repeat domain, a nucleic acid molecule encoding the designed repeat domain or the protein, or a pharmaceutical composition comprising i) the designed repeat domain, ii) the protein, iii) a nucleic acid molecule encoding the designed repeat domain or the protein, or iv) a combination thereof.


SEQUENCE LISTING (100 sequences)

SEQ ID NO: 1 - protein, 124 aa, synthetic construct
  source 1..124: note = Parent ankyrin repeat domain
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAQDKSGKTPADLAADAGHEDIAEVLQKAA

SEQ ID NO: 2 - protein, 157 aa, synthetic construct
  source 1..157: note = Parent ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAA

SEQ ID NO: 3 - protein, 124 aa, synthetic construct
  source 1..124: note = CD3-binding ankyrin repeat domain
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 4 - protein, 157 aa, synthetic construct
  source 1..157: note = CD123-binding ankyrin repeat domain
DLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAA

SEQ ID NO: 5 - protein, 124 aa, synthetic construct
  source 1..124: note = Non-binding DARPin domain
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 6 - protein, 157 aa, synthetic construct
  source 1..157: note = Designed ankyrin repeat domain
DLDKKLLEAARAGQDDEVRILLKAGADVNAKDSTGWTPLHLAAPWGHPEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAA

SEQ ID NO: 7 - protein, 157 aa, synthetic construct
  source 1..157: note = Designed ankyrin repeat domain
DLDKKLLEAARAGQDDEVRILLKAGADVNAKDSTGWTPLHLAAPWGHPEIVEVLLKAGADVNAKDVYGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 8 - protein, 157 aa, synthetic construct
  source 1..157: note = Designed ankyrin repeat domain
DLDKKLLEAARAGQDDEVRILLKAGADVNAKDSTGWTPLHLAAPWGHPEIVEVLLKAGADVNAKDFYGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 9 - protein, 157 aa, synthetic construct
  source 1..157: note = Designed ankyrin repeat domain
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 10 - protein, 157 aa, synthetic construct
  source 1..157: note = Designed ankyrin repeat domain
DLGKKLLQAARAGQLDEVRELLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 11 - protein, 190 aa, synthetic construct
  source 1..190: note = Designed ankyrin repeat domain
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAA

SEQ ID NO: 12 - protein, 190 aa, synthetic construct
  source 1..190: note = Designed ankyrin repeat domain
DLDKKLLEAARAGQDDEVRILLKAGADVNAKDSTGWTPLHLAAPWGHPEIVEVLLKAGADVNAKDFKGWTPLHLAAAAGHLEIVEVLLKAGADVNAKDVYGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 13 - protein, 190 aa, synthetic construct
  source 1..190: note = Designed ankyrin repeat domain
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDVYGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 14 - protein, 10 aa, synthetic construct
  source 1..10: note = His-tag
MRGSHHHHHH

SEQ ID NO: 15 - protein, 305 aa, synthetic construct
  source 1..305: note = Multi-domain ankyrin repeat protein
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLDKKLLEAARAGQDDEVRILLKAGADVNAKDSTGWTPLHLAAPWGHPEIVEVLLKAGADVNAKDFYGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 16 - protein, 305 aa, synthetic construct
  source 1..305: note = Multi-domain ankyrin repeat protein
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 17 - protein, 305 aa, synthetic construct
  source 1..305: note = Multi-domain ankyrin repeat protein
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLGKKLLQAARAGQLDEVRELLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 18 - protein, 338 aa, synthetic construct
  source 1..338: note = Multi-domain ankyrin repeat protein
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAA

SEQ ID NO: 19 - protein, 453 aa, synthetic construct
  source 1..453: note = Multi-domain ankyrin repeat protein
DLDKKLLEAARAGQDDEVRILLKAGADVNAKDSTGWTPLHLAAPWGHPEIVEVLLKAGADVNAKDFYGWTPLHLAAAAGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 20 - protein, 453 aa, synthetic construct
  source 1..453: note = Multi-domain ankyrin repeat protein
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 21 - protein, 453 aa, synthetic construct
  source 1..453: note = Multi-domain ankyrin repeat protein
DLGKKLLQAARAGQLDEVRELLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 22 - protein, 486 aa, synthetic construct
  source 1..486: note = Multi-domain ankyrin repeat protein
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 23 - protein, 486 aa, synthetic construct
  source 1..486: note = Multi-domain ankyrin repeat protein
DLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLDKKLLEAARAGQDDEVRILLKAGADVNAKDSTGWTPLHLAAPWGHPEIVEVLLKAGADVNAKDFYGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 24 - protein, 486 aa, synthetic construct
  source 1..486: note = Multi-domain ankyrin repeat protein
DLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 25 - protein, 486 aa, synthetic construct
  source 1..486: note = Multi-domain ankyrin repeat protein
DLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLGKKLLQAARAGQLDEVRELLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAA

SEQ ID NO: 26 - protein, 519 aa, synthetic construct
  source 1..519: note = Multi-domain ankyrin repeat protein
DLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAA

SEQ ID NO: 27 - protein, 486 aa, synthetic construct
  source 1..486: note = Multi-domain ankyrin repeat protein
DLDKKLLEAARAGQDDEVRILLKAGADVNAKDSTGWTPLHLAAPWGHPEIVEVLLKAGADVNAKDFYGWTPLHLAAAAGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 28 - protein, 486 aa, synthetic construct
  source 1..486: note = Multi-domain ankyrin repeat protein
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 29 - protein, 486 aa, synthetic construct
  source 1..486: note = Multi-domain ankyrin repeat protein
DLGKKLLQAARAGQLDEVRELLKAGADVNAKDSTGWTPLHLAAPWGHPEIVEVLLKAGADVNAKDFQGWTPLHLAAAAGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 30 - protein, 519 aa, synthetic construct
  source 1..519: note = Multi-domain ankyrin repeat protein
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 31 - protein, 305 aa, synthetic construct
  source 1..305: note = Multi-domain ankyrin repeat protein
DLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 32 - protein, 453 aa, synthetic construct
  source 1..453: note = Multi-domain ankyrin repeat protein
DLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSPTPTPTTPTPTPTTPTPTPTGSDLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAQDKSGKTPADLAADAGHEDIAEVLQKAA

SEQ ID NO: 33 - protein, 453 aa, synthetic construct
  source 1..453: note = Multi-domain ankyrin repeat protein
DLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAQDKSGKTPADLAADAGHEDIAEVLQKAAGSPTPTPTTPTPTPTTPTPTPTGSDLGKKLLQAARAGQLDEVRELLKAGADVNAKDQYGRTPLHLAAIKGHLEIVEVLLKAGADVNAKDSLGYTPLHLAAVEGPLEIVEVLLKAGADVNAKDAYGQTPLHIAAAWGHLEIVEVLLKAVADVNAQDKSGKTPADLAARAGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 34 - protein, 12 aa, synthetic construct
  source 1..12: note = His-tag
MRGSHHHHHHGS

SEQ ID NO: 35 - protein, 30 aa, synthetic construct
  source 1..30: note = N-terminal capping module
DLDKKLLEAARAGQDDEVRILLKAGADVNA

SEQ ID NO: 36 - protein, 30 aa, synthetic construct
  source 1..30: note = N-terminal capping module
DLGKKLLQAARAGQLDEVRELLKAGADVNA

SEQ ID NO: 37 - protein, 28 aa, synthetic construct
  source 1..28: note = C-terminal capping module
QDKSGKTPADLAADAGHEDIAEVLQKAA

SEQ ID NO: 38 - protein, 28 aa, synthetic construct
  source 1..28: note = C-terminal capping module
QDKSGKTPADLAARAGHQDIAEVLQKAA

SEQ ID NO: 39 - protein, 33 aa, synthetic construct
  source 1..33: note = Ankyrin repeat module
KDAKGLTPLHLAAYHGHLEIVEVLLKAGADVNA

SEQ ID NO: 40 - protein, 33 aa, synthetic construct
  source 1..33: note = Ankyrin repeat module
KDSTGWTPLHLAAPWGHPEIVEVLLKAGADVNA

SEQ ID NO: 41 - protein, 33 aa, synthetic construct
  source 1..33: note = Ankyrin repeat module
KDFQGWTPLHLAAAAGHLEIVEVLLKAGADVNA

SEQ ID NO: 42 - protein, 33 aa, synthetic construct
  source 1..33: note = Ankyrin repeat module
KDVYGWTPLHIAAASGHLEIVEVLLKAGADVNA

SEQ ID NO: 43 - protein, 33 aa, synthetic construct
  source 1..33: note = Ankyrin repeat module
KDVYGWTPLHLAAAAGHLEIVEVLLKAGADVNA

SEQ ID NO: 44 - protein, 33 aa, synthetic construct
  source 1..33: note = Ankyrin repeat module
KDFYGWTPLHLAAAAGHLEIVEVLLKAGADVNA

SEQ ID NO: 45 - protein, 33 aa, synthetic construct
  source 1..33: note = Ankyrin repeat module
KDFKGWTPLHLAAAAGHLEIVEVLLKAGADVNA

SEQ ID NO: 46 - protein, 33 aa, synthetic construct
  source 1..33: note = Ankyrin repeat module
KDWLGWTPLHLAASHGHLEIVEVLLKAGADVNA

SEQ ID NO: 47 - protein, 30 aa, synthetic construct
  source 1..30: note = N-terminal capping module
DLGKKLLEAARAGQDDEVRELLKAGADVNA

SEQ ID NO: 48 - protein, 30 aa, synthetic construct
  source 1..30: note = N-terminal capping module
  VARIANT 4..12: note = X can be any naturally occuring amino acid
DLGXKLLEAAXXGQDDEVRELLKAGADVNA

SEQ ID NO: 49 - protein, 30 aa, synthetic construct
  source 1..30: note = N-terminal capping module
  VARIANT 4..23: note = X can be any naturally occuring amino acid
DLGXKLLEAAXXGQDDXVRXLLXAGADVNA

SEQ ID NO: 50 - protein, 30 aa, synthetic construct
  source 1..30: note = N-terminal capping module
  VARIANT 4..24: note = X can be any naturally occuring amino acid
DLGXXLLEAAXXGQDDXVRXLXXXGADVNA

SEQ ID NO: 51 - protein, 30 aa, synthetic construct
  source 1..30: note = N-terminal capping module
  VARIANT 1..24: note = X can be any naturally occuring amino acid
XXXXXLLEAAXXGXDXXXXXXXXXGADVNA

SEQ ID NO: 52 - protein, 30 aa, synthetic construct
  source 1..30: note = N-terminal capping module
  VARIANT 1..24: note = X can be any naturally occuring amino acid
XXXXXXXEXXXXXXDXXXXXXXXXGADVNA

SEQ ID NO: 53 - protein, 30 aa, synthetic construct
  source 1..30: note = N-terminal capping module
DLGSKLLQAARAGQLDTVRTLLQAGADVNA

SEQ ID NO: 54 - protein, 30 aa, synthetic construct
  source 1..30: note = N-terminal capping module
  VARIANT 4..12: note = X can be any naturally occuring amino acid
DLGXKLLQAAXXGQLDEVRELLKAGADVNA

SEQ ID NO: 55 - protein, 30 aa, synthetic construct
  source 1..30: note = N-terminal capping module
  VARIANT 4..23: note = X can be any naturally occuring amino acid
DLGXKLLQAAXXGQLDXVRXLLXAGADVNA

SEQ ID NO: 56 - protein, 30 aa, synthetic construct
  source 1..30: note = N-terminal capping module
  VARIANT 4..24: note = X can be any naturally occuring amino acid
DLGXXLLQAAXXGQLDXVRXLXXXGADVNA

SEQ ID NO: 57 - protein, 30 aa, synthetic construct
  source 1..30: note = N-terminal capping module
  VARIANT 1..24: note = X can be any naturally occuring amino acid
XXXXXLLQAAXXGXLXXXXXXXXXGADVNA

SEQ ID NO: 58 - protein, 30 aa, synthetic construct
  source 1..30: note = N-terminal capping module
  VARIANT 1..24: note = X can be any naturally occuring amino acid
XXXXXXXQXXXXXXLXXXXXXXXXGADVNA

SEQ ID NO: 59 - protein, 24 aa, synthetic construct
  source 1..24: note = N-terminal capping module
DLGKKLLQAARAGQLDEVRELLKA

SEQ ID NO: 60 - protein, 24 aa, synthetic construct
  source 1..24: note = N-terminal capping module
DLGSKLLQAARAGQLDTVRTLLQA

SEQ ID NO: 61 - protein, 24 aa, synthetic construct
  source 1..24: note = N-terminal capping module
  VARIANT 4..12: note = X can be any naturally occuring amino acid
DLGXKLLQAAXXGQLDEVRELLKA

SEQ ID NO: 62 - protein, 24 aa, synthetic construct
  source 1..24: note = N-terminal capping module
  VARIANT 4..23: note = X can be any naturally occuring amino acid
DLGXKLLQAAXXGQLDXVRXLLXA

SEQ ID NO: 63 - protein, 24 aa, synthetic construct
  source 1..24: note = N-terminal capping module
  VARIANT 4..24: note = X can be any naturally occuring amino acid
DLGXXLLQAAXXGQLDXVRXLXXX

SEQ ID NO: 64 - protein, 28 aa, synthetic construct
  source 1..28: note = C-terminal capping module
  VARIANT 1..15: note = X can be any naturally occuring amino acid
XDXXGXTPADXAADXGHEDIAEVLQKAA

SEQ ID NO: 65 - protein, 28 aa, synthetic construct
  source 1..28: note = C-terminal capping module
  VARIANT 1..26: note = X can be any naturally occuring amino acid
XDXXGXTPADXAADXGHEXIAXVLQXAA

SEQ ID NO: 66 - protein, 28 aa, synthetic construct
  source 1..28: note = C-terminal capping module
  VARIANT 1..26: note = X can be any naturally occuring amino acid
XDXXGXTPXXXAADXGXEXXXXXXXXAA

SEQ ID NO: 67 - protein, 28 aa, synthetic construct
  source 1..28: note = C-terminal capping module
  VARIANT 1..28: note = X can be any naturally occuring amino acid
XDXXGXTPXXXXADXXXEXXXXXXXXXX

SEQ ID NO: 68 - protein, 28 aa, synthetic construct
  source 1..28: note = C-terminal capping module
  VARIANT 1..26: note = X can be any naturally occuring amino acid
XDXXGXTPXXXXXDXXXEXXXXXXXXAA

SEQ ID NO: 69 - protein, 28 aa, synthetic construct
  source 1..28: note = C-terminal capping module
QDTQGTTPADLAARAGHQQIASVLQQAA

SEQ ID NO: 70 - protein, 28 aa, synthetic construct
  source 1..28: note = C-terminal capping module
  VARIANT 1..15: note = X can be any naturally occuring amino acid
XDXXGXTPADXAARXGHQDIAEVLQKAA

SEQ ID NO: 71 - protein, 28 aa, synthetic construct
  source 1..28: note = C-terminal capping module
  VARIANT 1..26: note = X can be any naturally occuring amino acid
XDXXGXTPADXAARXGHQXIAXVLQXAA

SEQ ID NO: 72 - protein, 28 aa, synthetic construct
  source 1..28: note = C-terminal capping module
  VARIANT 1..26: note = X can be any naturally occuring amino acid
XDXXGXTPXXXAARXGXQXXXXXXXXAA

SEQ ID NO: 73 - protein, 28 aa, synthetic construct
  source 1..28: note = C-terminal capping module
  VARIANT 1..28: note = X can be any naturally occuring amino acid
XDXXGXTPXXXXARXXXQXXXXXXXXXX

SEQ ID NO: 74 - protein, 28 aa, synthetic construct
  source 1..28: note = C-terminal capping module
  VARIANT 1..26: note = X can be any naturally occuring amino acid
XDXXGXTPXXXXXRXXXQXXXXXXXXAA

SEQ ID NO: 75 - protein, 33 aa, synthetic construct
  source 1..33: note = Ankyrin repeat module
  VARIANT 3..15: note = X can be any naturally occuring amino acid
KDXXGXTPLHXAAXXGHLEIVEVLLKAGADVNA

SEQ ID NO: 76 - protein, 33 aa, synthetic construct
  source 1..33: note = Ankyrin repeat module
  VARIANT 3..15: note = X can be any naturally occuring amino acid
KDXXGXTPLHXAAXXGHPEIVEVLLKAGADVNA

SEQ ID NO: 77 - protein, 33 aa, synthetic construct
  source 1..33: note = Ankyrin repeat module
  VARIANT 1..27: note = X can be any naturally occuring amino acid
XDXXGXTPLHXAAXXGHLEIVEVLLKXGADVNA

SEQ ID NO: 78 - protein, 33 aa, synthetic construct
  source 1..33: note = Ankyrin repeat module
  VARIANT 1..27: note = X can be any naturally occuring amino acid
XDXXGXTPLHXAAXXGHPEIVEVLLKXGADVNA

SEQ ID NO: 79 - protein, 33 aa, synthetic construct
  source 1..33: note = Ankyrin repeat module
  VARIANT 1..27: note = X can be any naturally occuring amino acid
XDXXGXTPLHLAAXXGHLEIVEVLLKXGADVNA

SEQ ID NO: 80 - protein, 33 aa, synthetic construct
  source 1..33: note = Ankyrin repeat module
  VARIANT 3..15: note = X can be any naturally occuring amino acid
KDXXGXTPLHLAAXXGHLEIVEVLLKAGADVNA

SEQ ID NO: 81 - protein, 33 aa, synthetic construct
  source 1..33: note = Ankyrin repeat module
  VARIANT 1..27: note = X can be any naturally occuring amino acid
XDXXGXTPLHXAXXXGXXXIVXVLLXXGADVNA

SEQ ID NO: 82 - protein, 24 aa, synthetic construct
  source 1..24: note = PT-rich peptide linker
GSPTPTPTTPTPTPTTPTPTPTGS

SEQ ID NO: 83 - protein, 30 aa, synthetic construct
  source 1..30: note = N-terminal capping module
DLGKKLLEAARAGQDDEVRILLKAGADVNA

SEQ ID NO: 84 - protein, 30 aa, synthetic construct
  source 1..30: note = N-terminal capping module
DLGKKLLEAARAGQDDEVRILLAAGADVNA

SEQ ID NO: 85 - protein, 30 aa, synthetic construct
  source 1..30: note = N-terminal capping module
  VARIANT 4..12: note = X can be any naturally occuring amino acid
  VARIANT 15: note = X is selected from the group consisting of Leu, Val, Ile, Ala, Met and Thr.
  VARIANT 20..24: note = X can be any naturally occuring amino acid
DLGXKLLXAAXXGQXDEVRXLLXXGADVNA

SEQ ID NO: 86 - protein, 157 aa, synthetic construct
  source 1..157: note = Parent ankyrin repeat domain
DLGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 87 - protein, 157 aa, synthetic construct
  source 1..157: note = Parent ankyrin repeat domain
DLGKKLLEAARAGQDDEVRILMANGADVNANDWFGITPLHLVVNNGHLEIIEVLLKYAADVNASDKSGWTPLHLAAYRGHLEIVEVLLKYGADVNAMDYQGYTPLHLAAEDGHLEIVEVLLKYGADVNAQDKFGKTAFDISIDNGNEDLAEILQKLN

SEQ ID NO: 88 - protein, 157 aa, synthetic construct
  source 1..157: note = Parent ankyrin repeat domain
DLGKKLLQAARAGQLDEVRELLKAGADVNAKDSQGRTPLHLAAFAGHLEIVEVLLKAGADVNAKDHAGWTPLHLAAYAGHLEIVEVLVKAGADVNAKDTEGKTPLHIAAWFGHLEIVEVLLKAGADVNAQDKSGKTPADLTARAGHQDIAEVLQKAA

SEQ ID NO: 89 - protein, 33 aa, synthetic construct
  source 1..33: note = Ankyrin repeat module
KDKDGTTPLHLAAREGHLEIVEVLLKAGADVNA

SEQ ID NO: 90 - protein, 289 aa, synthetic construct
  source 1..289: note = Designed ankyrin repeat domain
DLGKKLLEAARAGQDDEVRILMANGADVNANDWFGITPLHLVVNNGHLEIIEVLLKYAADVNASDKSGWTPLHLAAYRGHLEIVEVLLKYGADVNAMDYQGYTPLHLAAEDGHLEIVEVLLKYGADVNAKDKFGKTPLHIAARAGHLEIVEVLLKAGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKAA

SEQ ID NO: 91 - protein, 289 aa, synthetic construct
  source 1..289: note = Designed ankyrin repeat domain
DLGKKLLEAARAGQDDEVRILMANGADVNANDWFGITPLHLVVNNGHLEIIEVLLKYAADVNASDKSGWTPLHLAAYRGHLEIVEVLLKYGADVNAMDYQGYTPLHLAAEDGHLEIVEVLLKYGADVNAKDKFGRTPLHIAARNGHLEIVEVLLKAGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKAA

SEQ ID NO: 92 - protein, 256 aa, synthetic construct
  source 1..256: note = Designed ankyrin repeat domain
DLGKKLLQAARAGQLDEVRELLKAGADVNAKDSQGRTPLHLAAFAGHLEIVEVLLKAGADVNAKDHAGWTPLHLAAYAGHLEIVEVLVKAGADVNAKDTEGKTPLHIAAWFGHLEIVEVLLKAGADVNAKDKSGQTPLHLAAWAGHLEIVEVLLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 93 - protein, 289 aa, synthetic construct
  source 1..289: note = Designed ankyrin repeat domain
DLGKKLLQAARAGQLDEVRELLKAGADVNAKDSQGRTPLHLAAFAGHLEIVEVLLKAGADVNAKDHAGWTPLHLAAYAGHLEIVEVLVKAGADVNAKDTEGKTPLHIAAWFGHLEIVEVLLKAGADVNAKDKSGKTPLHLAARAGHLEIVEVLLKAGADVNAKDNSGQTPLHEAAWAGHLEIVEVLLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 94 - protein, 289 aa, synthetic construct
  source 1..289: note = Designed ankyrin repeat domain
DLGKKLLQAARAGQLDEVRELLKAGADVNAKDSQGRTPLHLAAFAGHLEIVEVLLKAGADVNAKDHAGWTPLHLAAYAGHLEIVEVLVKAGADVNAKDTEGKTPLHIAAWFGHLEIVEVLLKAGADVNAKDKSGKTPLHLAARAGHLEIVEVLLKAGADVNAKDNSGQTPLHLAAWAGHLEIVEVLLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 95 - protein, 275 aa, synthetic construct
  source 1..275: note = Aminoglycoside phosphotransferase (APH)
MRGSHHHHHHGSAKMRISPELKKLIEKYRSVKDTEGMSPAKVYKLVGENENLYLKMTDSRYKGTTYDVEREKDMMLWLEGKLPVPKVLHFERHDGWSNLLMSEADGVLCSEEYEDEQSPEKIIELYAECIRLFHSIDISDCPYTNSLDSRLAELDYLLNNDLADVDSENWEEDTPFKDPRELYDFLKTEKPEEELVFSHGDLGDSNIFVKDGKVSGFIDLGRSGRADKWYDIAFCVRSIREDIGEEQYVELFFDLLGIKPDWEKIKYYILLDELF

SEQ ID NO: 96 - protein, 407 aa, synthetic construct
  source 1..407: note = Maltose Binding Protein (MBP)
MAGLNDIFEAQKIEWHEGSMGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGSGGTPGRPAAQASHHHHHH

SEQ ID NO: 97 - protein, 30 aa, synthetic construct
  source 1..30: note = N-terminal capping module
  VARIANT 4..15: note = X can be any naturally occuring amino acid
DLGXKLLXAAXXGQXDEVRELLKAGADVNA

SEQ ID NO: 98 - protein, 30 aa, synthetic construct
  source 1..30: note = N-terminal capping module
  VARIANT 4..23: note = X can be any naturally occuring amino acid
DLGXKLLXAAXXGQXDXVRXLLXAGADVNA

SEQ ID NO: 99 - protein, 30 aa, synthetic construct
  source 1..30: note = N-terminal capping molecule
  VARIANT 4..24: note = X can be any naturally occuring amino acid
DLGXXLLXAAXXGQXDXVRXLXXXGADVNA

SEQ ID NO: 100 - protein, 30 aa, synthetic construct
  source 1..30: note = N-terminal capping module
  VARIANT 1..24: note = X can be any naturally occuring amino acid
XXXXXLLXAAXXGXXXXXXXXXXXGADVNA